(19) United States
(12) Patent Application Publication
(10) Pub. No.: US 2014/0056952 A1
(43) Pub. Date: Feb. 27, 2014

(54) METHODS AND COATINGS FOR TREATING BIOFILMS

(71) Applicant: President and Fellows of Harvard College, Cambridge, MA (US)

(72) Inventors: Richard LOSICK, Lexington, MA (US); Illana KOLODKIN-GAL, Cambridge, MA (US); Jon CLARDY, Jamaica Plain, MA (US); Shugeng CAO, Waltham, MA (US); Matt CABEEN, Dorchester, MA (US); Roberto KOLTER, Cambridge, MA (US); Liraz CHAI, Giv`atayim (IL); Thomas BÖTTCHER, Maisach (DE)

(73) Assignee: President and Fellows of Harvard College, Cambridge, MA (US)

(21) Appl. No.: 14/070,858

(22) Filed: Nov. 4, 2013

Related U.S. Application Data
(63) Continuation of application No. PCT/US12/36668, filed on May 4, 2012.
(60) Provisional application No. 61/482,523, filed on May 4, 2011.
(60) Provisional application No. 61/482,522, filed on May 4, 2011.
(60) Provisional application No. 61/591,601, filed on Jan. 27, 2012.

(51) Int. Cl. A01N 43/36; C07C 211/13; C07C 211/09; C07C 211/11; C07C 217/42; A01N 37/44; C07D 259/00; C07D 207/34; C07D 207/337; A01N 33/26; C07C 237/06; A01N 33/04; A01N 43/713

(52) U.S. Cl. CPC A01N 43/36; A01N 33/04; C07C 211/13; C07C 211/09; C07C 211/11; C07C 217/42; A01N 43/713; C07D 259/00; C07D 207/34; C07D 207/337; A01N 33/26; C07C 237/06; A01N 37/44
USPC 424/400; 514/674; 514/673; 514/670; 514/183; 514/423; 514/427; 514/614; 514/567

(57) ABSTRACT

Methods of treating or reducing biofilms, treating a biofilm-related disorder, and preventing biofilm formation using polyamines is described.

### CROSS REFERENCE TO RELATED APPLICATIONS

This application is a continuation of International Application No. PCT/US12/36668, filed May 4, 2012, which claims the benefit of U.S. Provisional Application No. 61/591,601, filed Jan. 27, 2012, U.S. Provisional Application No. 61/482,523, filed May 4, 2011, and U.S. Provisional Application No. 61/482,522, filed May 4, 2011, the disclosures of all of which are hereby incorporated by reference herein.

### STATEMENT OF GOVERNMENT RIGHTS

This invention was made with Government support under National Institutes of Health awards GM18568, CA24487, GM86258, GM058213, GM082137, and AI057159. The Government has certain rights in the invention.

### BACKGROUND

Biofilms are communities of cells that settle and proliferate on surfaces and are covered by an exopolymer matrix. They are slow-growing and many are in the stationary phase of growth. A hallmark of biofilms is an extracellular matrix typically consisting of protein, exopolysaccharide and sometimes DNA, that holds the cells together in the community. They can be formed by most, if not all, pathogens. According to the CDC, 65% of all infections in the United States are caused by biofilms that can be formed by common pathogens. Biofilms are also found in industrial settings, such as in drinking water distribution systems.

### SUMMARY

It has been discovered that certain polyamines inhibit biofilm formation and trigger biofilm disassembly. Aspects of this disclosure feature methods of treating, reducing, inhibiting biofilm formation by bacteria, and triggering biofilm disassembly. In some embodiments, the method comprises contacting a surface with a composition comprising an effective amount of a polyamine, thereby treating, reducing or inhibiting formation of the biofilm or triggering disassembly of the biofilm.

It has been discovered that the structure of the polyamine contributes to its ability to inhibit biofirm formation and trigger disassembly of a biofilm. In certain embodiments, the polyamine has at least three amino groups separated by three atoms either in a straight chain or cyclic molecule. In certain embodiments, the amino groups are ionizable. In certain embodiments, the amino groups are positively charged. In some embodiments, the polyamines are branched. In some embodiments, the polyamines are linear. In certain embodiments, the polyamine has Formula (I),

HR₇N-(M-N(R₇)—)ₓ-M-N(R₇)—Y—N(R₇)-(M-N(R₇)—)ₓ-M-NR₇H (I)

wherein

M is —C(R₁R₂)C(R₃R₄)C(R₅R₆)—;

each R₁, R₂, R₃, R₄, R₅, and R₆ is H, C₁-C₁₂ alkyl, C₁-C₁₂ alkenyl, C₂-C₁₂ alkynyl, C₁-C₁₂ alkoxy, alkaryl, aryl, or heteroaryl, so that each R₁, R₂, R₃, R₄, R₅, and R₆ may be the same or different;

each R₇ is H, C₁-C₁₆ alkyl, C₂-C₁₆ alkenyl, C₂-C₁₆ alkynyl, aryl, heteroaryl, or C₇₋₂₂ aralkyl, so that each R₇ may be the same or different;

Y is a moiety that interrupts the polyamine chain and is C₁-C₁₂ alkyl, C₁-C₁₂ alkenyl, C₂-C₁₂ alkynyl, C₁-C₁₂ alkoxy, alkaryl, aryl, or heteroaryl, polymer block, or oligomer block; and

each x is greater than or equal to 1.

In some embodiments, the polyamines can have Formula (Ia), where some R₇ groups in Formula (I) is replaced with R₇ₐ, which is defined as H, C₁₋₈ ω-amino alkyl, C₂₋₈ ω-amino alkenyl, C₂₋₈ ω-amino alkynyl, amino alkaryl, amino aryl, N-heteroaryl. In such cases, the polyamines have Formula (Ia),

HR₇N-(M-N(R₇ₐ)—)ₓ-M-N(R₇)—Y—N(R₇ₐ)-(M-N(R₇ₐ)—)ₓ-M-NR₇H (Ia)

wherein

M is —C(R₁R₂)C(R₃R₄)C(R₅R₆)—;

each R₁, R₂, R₃, R₄, R₅, and R₆ is H, C₁-C₁₂ alkyl, C₁-C₁₂ alkenyl, C₂-C₁₂ alkynyl, C₁-C₁₂ alkoxy, alkaryl, aryl, or heteroaryl, so that each R₁, R₂, R₃, R₄, R₅, and R₆ may be the same or different;

each R₇ is H, C₁-C₁₆ alkyl, C₂-C₁₆ alkenyl, C₂-C₁₆ alkynyl, aryl, heteroaryl, or C₇₋₂₂ aralkyl, so that each R₇ may be the same or different;

each R₇ₐ is H, C₁₋₈ ω-amino alkyl, C₂₋₈ ω-amino alkenyl, C₂₋₈ ω-amino alkynyl, amino alkaryl, amino aryl, N-heteroaryl, so that each R₇ may be the same or different;

Y is a moiety that interrupts the polyamine chain and is C₁-C₁₂ alkyl, C₁-C₁₂ alkenyl, C₂-C₁₂ alkynyl, C₁-C₁₂ alkoxy, alkaryl, aryl, or heteroaryl, polymer block, or oligomer block; and

each x is greater than or equal to 1.

Compounds of Formulae (I) may be acyclic or cyclic. If a compound of Formulae (I) is cyclic, the terminal NHR₁ groups of Formula (I) are NR₁ and form a ring where each NR₁ covalently bonds to one M group, thereby forming a ring.

In certain embodiments, the polymer or oligomer blocks of Y can comprise one or more of carbonyl, epoxy, ester, carboxyl, amine, amide, imine, imide, or glycol.

In some embodiments, the polyamine has Formula (II),

Hₐ-[R₇N-(L-N(R₈)—)ₓ-L-NH—P_{b}]_{c} (II),

wherein

each P is R₇ or Q or L;

each L is M or —C(R₁R₂)—X—C(R₅R₆)— or

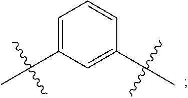 [SMILES: *c1cccc(*)c1]

each M is —C(R₁R₂)C(R₃R₄)C(R₅R₆)—;

each R₁, R₂, R₃, R₄, R₅, and R₆ is H, C₁-C₁₂ alkyl, C₁-C₁₂ alkenyl, C₂-C₁₂ alkynyl, C₁-C₁₂ alkoxy, alkaryl, aryl, or heteroaryl, so that each R₁, R₂, R₃, R₄, R₅, and R₆ may be the same or different;

each R₇ is H, C₁-C₁₆ alkyl, C₂-C₁₆ alkenyl, C₂-C₁₆ alkynyl, aryl, heteroaryl, or C₇₋₂₂ aralkyl which may be substituted on the aryl ring, or R₈ so that each R₇ may be the same or different, wherein R₇ is R₈ when the polyamine is cyclic;

each R₈ is H, C₁₋₈ ω-amino alkyl, C₂₋₈ ω-amino alkenyl, C₂₋₈ ω-amino alkynyl, amino alkaryl, amino aryl, N-heteroaryl;

each X is —NH—, —O—, —N(R₈)—, or S;

each Q is C₁₋₃₀ alkyl, C₂₋₃₀ alkenyl, C₂₋₃₀ alkynyl, alkaryl, aryl, heteroaryl, which may be substituted and where the alkyl, alkenyl, alkynyl, and alkaryl may be interrupted by one or more heteroatoms such as N, O, or S;

a is 0 or 1, wherein when a is 0 the polyamine is cyclic and when a is 1 the polyamine is linear or branched;

each b is 0 or 1;

c is greater than or equal to 1; and

each x is greater than or equal to 1.

In some embodiments of Formula (II), a is 1 and the polyamine is linear or branched. In certain embodiments of Formula (II), a is 1, b is 1, c is 1, L is M, P is R₇, R₇ and R₈ are hydrogen, and the polyamine has Formula (IIa),

H₂N-(M-NH)ₓ-M-NH₂ (IIa).

In certain embodiments a is 0 and compounds of Formula (II) may be cyclic. In some embodiments, a is 0, b is 1, P is Q, and the ring is formed by Q bonding to the terminal NR₇ group. In some embodiments, a is 0, b is 1, P is L, L is M, R₇ and R₈ are hydrogen, the terminal NR₇ group (where R₇ is hydrogen) bonds to the terminal L group (which is M), and the cyclic polyamine has Formula (IIb),

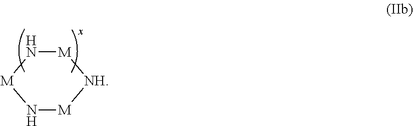

Compounds of Formulae (I), (Ia), (II), and (IIa) may be linear or branched.

In some embodiments of compounds of Formulae (I), (Ia), (II), (IIa), and (IIb), x may be 1. In some embodiments of compounds of Formulae (I), (Ia), (II), (IIa), and (IIb), x may be 2. In other embodiments of compounds of the above Formulae (I), (Ia), (II), (IIa), and (IIb), x may be 3. In still other embodiments of compounds of the above Formulae (I), (Ia), (II), (IIa), and (IIb), x may be 4. In still other embodiments of compounds of the above Formulae (I), (Ia), (II), (IIa), and (IIb), x may be 5. In further embodiments of compounds of the above Formulae (I), (Ia), (II), (IIa), and (IIb), x may be greater than 5.

In some embodiments, M is —CH₂CH₂CH₂—. In some embodiments, R₇ is —(CH₂)₁₁CH₃. In other embodiments, R₇ is hydrogen. In some embodiments, R₈ is hydrogen. In other embodiments, R₈ is —(CH₂)₃NH₂.

In some embodiments, at least one of R₁, R₂, R₃, R₄, R₅, or R₆ is C₁alkenyl and the other R group attached to the same carbon atom does not exist. For example, if R₁ is C₁-alkenyl, then R₂ does not exist.

In some embodiments, L is

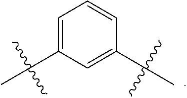 [SMILES: *c1cccc(*)c1]

Compound i in Table 1 exemplifies such an embodiment.

In some embodiments, the composition comprises norspermidine (also known as N-(3-aminopropyl)propane-1,3-diamine), norspermine (N′-[3-(3-aminopropylamino)propyl]propane-1,3-diamine), 1,5,9-triazacyclododecane, or a combination thereof. In further embodiments, the composition comprises two or more of norspermidine, norspermine, and 1,5,9-triazacyclododecane. In certain embodiments, the polyamine has Formulae (I), (Ia), (II), (IIa), or (IIb). In other embodiments, the polyamine is a compound in Table 1.

In some embodiments, the bacteria are Gram-negative or Gram-positive bacteria. In particular embodiments, the bacteria are Bacillus, Staphylococcus, E. coli, or Pseudomonas bacteria. In some embodiments, the bacteria are mycobacteria.

In one or more other embodiments, the surface comprises industrial equipment, plumbing systems, bodies of water, household surfaces, textiles and paper.

In certain embodiments, the method comprises contacting a surface with a composition comprising an effective amount of a polyamine combined with a D-amino acid, thereby treating, reducing, inhibiting formation of the biofilm, or triggering disassembly of the biofilm.

In other aspects, this disclosure features compositions, such as industrial, therapeutic or pharmaceutical compositions, comprising one or more polyamines. In some embodiments, the composition comprises at least one polyamine of Formulae (I), (Ia), (II), (IIa), or (IIb).

In other aspects, the invention features compositions comprising one or more polyamines combined with one or more D-amino acids. In some embodiments, the composition comprises at least one polyamine of Formulae (I), (Ia), (II), (IIa), or (IIb). In some embodiments, the polyamine has Formulae (I), (Ia), (II), (IIa), or (IIb) and the D-amino acid is selected from the group consisting of D-alanine, D-cysteine, D-aspartic acid, D-glutamic acid, D-histidine, D-isoleucine, D-lysine, D-leucine, D-asparagine, D-proline, D-glutamine, D-arginine, D-serine, D-threonine, D-valine, D-tryptophan, D-tyrosine, and a combination thereof, or the D-amino acid is a synergistic combination of two or more D-amino acids selected from the group consisting of D-alanine, D-cysteine, D-aspartic acid, D-glutamic acid, D-phenylalanine, D-histidine, D-isoleucine, D-lysine, D-leucine, D-methionine, D-asparagine, D-proline, D-glutamine, D-arginine, D-serine, D-threonine, D-valine, D-tryptophan, and D-tyrosine.

In some embodiments, the D-amino acid is D-tyrosine or the combination of D-amino acids comprises D-tyrosine. In other embodiments, the composition further comprises one or more of D proline and D phenylalanine. In other embodiments, the composition further comprises one or more of D-leucine, D-tryptophan, and D-methionine. In some embodiments, the composition further comprises one or more of D-alanine, D-cysteine, D-aspartic acid, D-glutamic acid, D-phenylalanine, D-histidine, D-isoleucine, D-lysine, D-leucine, D-methionine, D-asparagine, D-proline, D-glutamine, D-arginine, D-serine, D-threonine, D-valine, D-tryptophan, and D-tyrosine.

One aspect of this disclosure is directed to methods of treating, reducing, or inhibiting biofilm formation by a biofilm forming bacteria, the method comprising contacting an article with a composition comprising an effective amount of at least one polyamine. In some embodiments, the polyamine is norspermidine, norspermine, 1,5,9-triazacyclododecane, or a combination thereof. In certain embodiments, the polyamine has Formulae (I), (Ia), (II), (IIa), or (IIb). In other embodiments, the polyamine is a compound in Table 1.

In some embodiments, the method comprises contacting an article with a composition comprising an effective amount of at least one polyamine and a D-amino acid, thereby treating, reducing or inhibiting formation of the biofilm. In some embodiments, the polyamine has Formulae (I), (Ia), (II), (IIa), or (IIb), or the polyamine is a compound in Table 1, or a combination of one or more compounds in Table 1, the D-amino acid is selected from the group consisting of D-alanine, D-cysteine, D-aspartic acid, D-glutamic acid, D-histidine, D-isoleucine, D-lysine, D-leucine, D-asparagine, D-proline, D-glutamine, D-arginine, D-serine, D-threonine, D-valine, D-tryptophan, D-tyrosine, and a combination thereof, or the D-amino acid is a synergistic combination of two or more D-amino acids selected from the group consisting of D-alanine, D-cysteine, D-aspartic acid, D-glutamic acid, D-phenylalanine, D-histidine, D-isoleucine, D-lysine, D-leucine, D-methionine, D-asparagine, D-proline, D-glutamine, D-arginine, D-serine, D-threonine, D-valine, D-tryptophan, and D-tyrosine.

In some embodiments, the article is selected from the group consisting of industrial equipment, plumbing systems, bodies of water, household surfaces, textiles and paper. In further embodiments, the article is one or more components involved in water condensate collection, water recirculation, sewerage transport, paper pulping and manufacture, and water processing and transport. In still other embodiments, the article is a drain, tub, kitchen appliance, countertop, shower curtain, grout, toilet, industrial food or beverage production facility, floor, boat, pier, oil platform, water intake port, sieve, water pipe, cooling system, or powerplant.

In some embodiments, the article is made from a material selected from the group consisting of metal, metal alloy, synthetic polymer, natural polymer, ceramic, wood, glass, leather, paper, fabric, non-metallic inorganics, composite materials and combinations thereof.

In other embodiments, contacting comprises applying a coating to the article, said coating comprising an effective amount of a polyamine. In some embodiments, contacting comprises applying a coating to the article, said coating comprising an effective amount of a polyamine and a D-amino acid. In further embodiments, the coating further comprises a binder. In some embodiments, the coating is accomplished by wicking, spraying, dipping, spin coating, laminating, painting, screening, extruding or drawing down a coating composition onto the surface. In other embodiments, contacting comprises introducing a polyamine, or a combination of a polyamine and D-amino acid, into a precursor material and processing the precursor material into the article impregnated with the polyamine, or combination of a polyamine and D-amino acid. In further embodiments, contacting comprising introducing a polyamine, or a combination of a polyamine and D-amino acid, into a liquid composition.

Another aspect of this disclosure is directed to coated articles resistant to biofilm formation, comprising an article comprising a coating on at least one exposed surface, the coating comprising an effective amount of at least one polyamine. In some embodiments, the polyamine has Formulae (I), (Ia), (II), (IIa), or (IIb). In other embodiments, the polyamine is a polyamine selected from Table 1. In some embodiments, the coating comprises a combination of polyamines. In certain embodiments, the combination of polyamines is a combination of one or more polyamines having Formulae (I), (Ia), (II), (IIa), or (IIb) or a combination of one or more polyamines from Table 1.

In some embodiments, the coating comprises at least one polyamine combined with a D-amino acid, thereby treating, reducing or inhibiting formation of the biofilm. In some embodiments, the polyamine has Formulae (I), (Ia), (II), (IIa), or (IIb) or is a compound in Table 1, or is a combination of one or more compounds in Table 1, the D-amino acid is selected from the group consisting of D-alanine, D-cysteine, D-aspartic acid, D-glutamic acid, D-histidine, D-isoleucine, D-lysine, D-leucine, D-asparagine, D-proline, D-glutamine, D-arginine, D-serine, D-threonine, D-valine, D-tryptophan, D-tyrosine, and a combination thereof, or the D-amino acid is a synergistic combination of two or more D-amino acids selected from the group consisting of D-alanine, D-cysteine, D-aspartic acid, D-glutamic acid, D-phenylalanine, D-histidine, D-isoleucine, D-lysine, D-leucine, D-methionine, D-asparagine, D-proline, D-glutamine, D-arginine, D-serine, D-threonine, D-valine, D-tryptophan, and D-tyrosine.

In some embodiments, the article is selected from the group consisting of industrial equipment, plumbing systems, bodies of water, household surfaces, textiles and paper. In other embodiments, the article is one or more components involved in water condensate collection, water recirculation, sewerage transport, paper pulping and manufacture, and water processing and transport. In further embodiments, the article is a drain, tub, kitchen appliance, countertop, shower curtain, grout, toilet, industrial food or beverage production facility, floor, boat, pier, oil platform, water intake port, sieve, water pipe, cooling system, or powerplant.

In some embodiments, the article is made from a material selected from the group consisting of metal, metal alloy, synthetic polymer, natural polymer, ceramic, wood, glass, leather, paper, fabric, non-metallic inorganics, composite materials and combinations thereof. In further embodiments, the coating further comprises a binder. In other embodiments, the coating further comprises a polymer and the D-amino acid is distributed in the polymer.

In some embodiments, the polyamine coating, or the combination of polyamine and D-amino acid coating, is formulated as a slow-release formulation.

Another aspect of this disclosure is directed to compositions resistant to biofilm formation, comprising a fluid base; and an effective amount of at least one polyamine. In some embodiments, the polyamine has Formulae (I), (Ia), (II), (IIa), or (IIb). In some embodiments, the polyamine is selected from the group of compounds in Table 1. In certain embodiments, the polyamine is combined with a D-amino acid or a combination of D-amino acids, distributed in the base, thereby treating, reducing, or inhibiting formation of the biofilm. In some embodiments, the D-amino acid is selected from the group consisting of D-alanine, D-cysteine, D-aspartic acid, D-glutamic acid, D-histidine, D-isoleucine, D-lysine, D-leucine, D-asparagine, D-proline, D-glutamine, D-arginine, D-serine, D-threonine, D-valine, D-tryptophan, D-tyrosine, and a combination thereof, or the combination of D-amino acids is a synergistic combination of two or more D-amino acids selected from the group consisting of D-alanine, D-cysteine, D-aspartic acid, D-glutamic acid, D-phenylalanine, D-histidine, D-isoleucine, D-lysine, D-leucine, D-methionine, D-asparagine, D-proline, D-glutamine, D-arginine, D-serine, D-threonine, D-valine, D-tryptophan, and D-tyrosine.

In some embodiments, the fluid base is selected from a liquid, gel, orpaste.

In some embodiments, the composition is selected from the group consisting of water, washing formulations, disinfecting formulations, paints and coating formulations.

Yet another aspect of this disclosure is directed to coating compositions comprising at least one polyamine. In some embodiments, the polyamine has Formula (I), (II), (IIa), or (IIb). In some embodiments, the polyamine is selected from the group of compounds in Table 1. In some embodiments, the composition comprises a combination of polyamines of Formulae (I), (Ia), (II), (IIa), or (IIb), or a combination of one or more polyamines selected from the group of compounds in Table 1.

In certain embodiments, the polyamine is combined with a D-amino acid or a combination of two or more D-amino acids, selected from the group consisting of D-tyrosine, D-leucine, D-methionine, and D-tryptophan, and at least one D-amino acid is a different D-amino acid selected from the group consisting of D-cysteine, D-aspartic acid, D-glutamic acid, D-phenylalanine, D-histidine, D-isoleucine, D-lysine, D-leucine, D-methionine, D-asparagine, D-proline, D-glutamine, D-arginine, D-serine, D-threonine, D-valine, D-tryptophan, and D-tyrosine, and a polymeric binder.

In some embodiments of the foregoing methods and compositions, the composition comprises a polyamine and D-tyrosine. In certain embodiments, the polyamine combined with D-tyrosine has Formulae (I), (Ia), (II), (IIa), or (IIb) or is selected from the group of compounds in Table 1, or is a combination of one or more compounds in Table 1. In other embodiments, the composition further comprises one or more of D-proline and D-phenylalanine. In still other embodiments, the composition further comprises one or more of D-leucine, D-tryptophan, and D-methionine. In still further embodiments, the composition further comprises one or more of D-alanine, D-cysteine, D-aspartic acid, D-glutamic acid, D-phenylalanine, D-histidine, D-isoleucine, D-lysine, D-leucine, D-methionine, D-asparagine, D-proline, D-glutamine, D-arginine, D-serine, D-threonine, D-valine, D-tryptophan, D-tyrosine.utamic acid, D-phenylalanine, D-histidine, D-isoleucine, D-lysine, D-leucine, D-asparagine, D-proline, D-glutamine, D-arginine, D-serine, D-threonine, D-valine, D-tryptophan, and D-tyrisone.

In some embodiments of the foregoing methods and compositions, the methods further comprise contacting the surface with a biocide. In some embodiments, the composition comprises polyhexamethylene biguanide, chlorhexidine, xylitol, triclosan, or chlorine dioxide.

In some embodiments of the foregoing methods, the formation of a biofilm is inhibited. In other embodiments, a previously formed biofilm is disrupted.

### BRIEF DESCRIPTION OF THE FIGURES

The following figures are presented for the purpose of illustration only, and are not intended to be limiting.

FIGS. 1A-E show the identification of norspermidine in conditioned medium from B. subtilis and the effect of norspermidine on pellicle formation.

FIG. 1A shows the result of growing cells in fresh medium to which had been added 20 μl of the 25%, 35% or 40% methanol eluates.

FIG. 1B shows the results of cells of NCBI3610 that were grown in fresh medium containing PBS buffer (control), norspermidine (100 μM), morpholine (100 μM) HPLC-purified fatty acid (˜100 μM), or spermidine (100 μM).

FIG. 1C shows the detection of norspermidine in pellicles.

FIGS. 1D & 1E shows the quantification of the biofilm-inhibiting activity of norspermidine and spermidine.

FIGS. 2A and 2B1-2B4 show the results of the testing of various concentrations of norspermidine on biofilms and the detection of norspermidine.

FIG. 2A shows the minimal biofilm inhibiting concentration of norspermidine. Pellicle formation of strain NCBI 3610 was tested in the presence of various concentrations of norspermidine as indicated.

FIG. 2B1-2B4 show the detection of norspermidine. 2B1: Norspermidine purchased from Sigma Aldrich was used a standard for the detection of norspermidine in the biofilm. Norspermidine was derivatized with Fmoc-Cl and the resulting Fmoc-norspermidine (RT: 10.1 min) was quantified using an Agilent LC/MS system. 2B2: The UV spectrum of the reaction product of Fmoc-Cl and norspermidine at 10 min. 2B3: Positive MS (798 Da) of the reaction product of Fmoc-Cl and norspermidine at 10.1 min. 2B4: Derivatization reaction of norspermidine with Fmoc-Cl.

FIG. 3A shows 7 day-old cultures of the wild type (WT), a mutant (ΔgbaT) blocked in norspermidine production (IKG623), a double mutant (ΔylmE AracX) blocked in D-amino acid production (IKG55) and a triple mutant (ΔgbaT ΔylmE ΔracX) blocked in the production of both (IKG625).

FIG. 3B show the effects of combinations of amino acids and norspermidine at indicated concentrations on biofilm formation.

FIG. 3C shows the results of quantifying the pellicle breakdown to see whether a combination of D-amino acids and norspermidine was more effective than either D-amino acids or norspermidine alone.

FIG. 4A shows that cells for a mutant for a homolog of the norspermidine decarboxylase gene yaaO are delayed in pellicle disassembly. NCBI 3610 (WT), a mutant for yaaO (IKG624), a mutant doubly deleted for ylmE and racX (IKG55) or a triple mutant for yaaO, ylmE and racX (IKG626) for were grown in 12-well plates and incubated for 7 days.

FIG. 4B shows 3-day-old cultures of the wild type (NCBI 3610), an exopolysaccharide mutant (ΔepsH; DS76), a TasA mutant (ΔtasA; FC55), and, as indicated, wild type and mutant strains grown in the presence of 25 μM norspermidine.

FIG. 5 shows phase contrast and fluorescence images of cells of the wild-type (WT; NCBI 3610) harvested from pellicles grown in the presence or absence (untreated) of norspermidine (25 μM) or a high concentration of spermidine (1 mM). The cells were washed in PBS and stained for exopolysaccharide with a conjugate of concanavalin A with Texas-Red.

FIG. 6A shows that concanavalin A-Texas Red stain is largely specific to exopolysaccharide. Fluorescence microscopy was carried out with 3-day-old standing cultures. Cells of wild type strain (NCBI 3610) and an eps mutant (DS76) were collected and stained for one hour as in Experimental Procedures. Cells were imaged using the indicated exposure times. Little or no staining was observed for the mutant except when image brightness was enhanced as shown in the enlargement or when the cells were stained for 150 minutes (data not shown). Images were collected using the automated software program SimplePCl.

FIG. 6B shows that TasA-mCherry is not released from norspermidine-treated cells. NCBI 3610 containing the tasA-mCherry fusion (DR30) was grown without shaking in a biofilm medium (upper row) or in the same medium applied with norspermidine (50 μM, lower row). Cells were washed in PBS, and visualized by fluorescence microscopy.

FIG. 7A shows the average hydrodynamic radii of the exopolysaccharide as measured by dynamic light scattering. Shown are the results obtained in the absence of polyamine (black), in the presence of norspermidine (white), and in the presence of spermidine (grey) with exopolysaccharide at the indicated concentrations and pH. Error bars represent the standard deviation of polymer radii among the polymers in a single sample.

FIG. 7B shows three different magnifications of representative fields showing exopolysaccharide alone (EPS) and exopolysaccharide that had been mixed with norspermidine (EPS+norspermidine) or with spermidine (EPS+spermidine).

FIG. 8A shows that norspermidine does not inhibit growth of B. subtilis at concentrations that block biofilm formation.

FIG. 8B shows that norspermidine does not inhibit expression of P_{epsA}-lacZ at concentrations that block biofilm formation. Strain FC5 (carrying P_{epsA}-lacZ) was grown in MSgg medium containing norspermidine (100 μM) with shaking or in untreated medium as control (NT).

FIG. 9A shows the compounds that were tested for biofilm-inhibiting activity.

FIG. 9B shows the effect of the numbered compounds on pellicle formation by NCBI3610. The compounds were tested at 200 μM.

FIGS. 9C & 9D show computer modeling of the interaction of norspermidine and spermidine with an acidic exopolysaccharide.

FIG. 10 shows the results of the examination of relationship of the structure and activity of the polyamines in B. subtilis.

FIG. 11 shows the computer modeling of norspermidine binding to a neutral exopolysaccharide. Two PGA stands can be aligned with norspermidine by alternating hydrogen bonds to the acetyl groups of PGA.

FIG. 12A shows the effect of the numbered compounds displayed in FIG. 9A on the formation of submerged biofilms by S. aureus strain SCO1. The compounds were tested at 500 μM. Biofilm formation was visualized by crystal violet staining of submerged biofilms.

FIG. 12B shows quantification of the effects of norspermidine, norspermine, spermine and spermidine as measured by crystal violet staining (see Experimental procedures).

FIG. 13A shows the effect of the numbered compounds shown in FIG. 9A on submerged biofilm formation by E. coli strain MC4100. The compounds were tested at 500 μM. Biofilm formation was visualized by crystal violet staining of submerged biofilms.

FIG. 13B shows quantification of the effects of norspermidine, norspermine, spermine and spermidine as measured by crystal violet staining (see Experimental procedures).

FIG. 14A shows the results of the examination of the relationship of the structure and activity of the polyamines in S. aureus. The effect of the numbered compounds on the formation of submerged biofilms by S. aureus strain SC01. Biofilm formation was visualized by crystal violet staining of submerged biofilms.

FIG. 14B shows the results of the examination of the relationship of the structure and activity of the polyamines in E. coli. The effect of the numbered compounds on the formation of submerged biofilms by E. coli strain MC4100. Biofilm formation was visualized by crystal violet staining of submerged biofilms.

FIG. 15 shows the inhibition of biofilm formation Bacillus subtilis in cells treated with (B) norspermidine and (E) norspermine as compared to the formation of biofilms in (A) cells not treated (“NT”) or treated with (C) spermidine or (D) speramide.

FIG. 16 is photographs of shows that cells treated with norspermidine (B) and norspermine (C) inhibit pellicle formation by B. subtilis as compared with untreated cells (A) and those treated with spermidine (D) or spermine (E).

FIGS. 17A and 17B shows that polyamines mediate pellicle disassembly by B. subtilis by comparison of an untreated cell culture (A) having an intact pellicle and a culture treated with norspermine (B) showing a disrupted pellicle.

FIGS. 18A, 18B, 18C, and 18D show that 1,5,9-triazacyclododecane inhibits biofilm formation by B. subtilis. FIGS. 18A and 18C are not treated with the polyamine. FIGS. 18B and 18D are treated with 1,5,9-triazacyclododecane.

FIG. 19AH shows that polyamines inhibit biofilm formation by Staphylococcus aureus.

FIGS. 20A-D shows that polyamines act synergistically with D-amino acids in inhibiting biofilm formation by Staphylococcus.

FIGS. 21A-H shows that polyamines inhibit biofilm formation by Pseudomonas aeruginosa.

FIG. 22 shows that polyamines inhibit biofilm formation by Proteus mirabilis. Biofilm formation was visualized by crystal violet staining of submerged biofilms.

### DETAILED DESCRIPTION

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims. As will be apparent to one of skill in the art, specific features and embodiments described herein can be combined with any other feature or embodiment.

### DEFINITIONS

The term “alkyl” refers to a hydrocarbon chain that may be a straight chain or branched chain. The chain may contain an indicated number of carbon atoms. For example, C₁-C₁₂ indicates that the group may have from 1 to 12 (inclusive) carbon atoms in it. An alkyl group can be substituted or unsubstituted. When substituted, one or more carbon atoms may be replaced with a heteroatom such as N, O, or S.

The term alkoxy refers to a straight or branched chain saturated or unsaturated hydrocarbon containing at least one oxygen atom. The chain may contain an indicated number of carbon atoms. For example, “C₁-C₁₂ alkoxy” indicates that the group may have from 1 to 12 (inclusive) carbon atoms and at least one oxygen atom. Examples of a C₁-C₁₂ alkoxy include, but are not limited to, methoxy, ethoxy, isopropoxy, butoxy, n-pentoxy, isopentoxy, neopentoxy, and hexoxy. An alkoxy group can be substituted or unsubstituted. When substituted, one or more carbon atoms may be replaced with a heteroatom such as N, O, or S.

The term alkenyl refers to a straight or branched chain hydrocarbon containing at least one carbon-carbon double bond. The chain may contain an indicated number of carbon atoms. For example, “C₁-C₁₂ alkenyl” indicates that the group may have from 1 to 12 (inclusive) carbon atoms and at least one carbon-carbon double bond. Exemplary such groups include, but are not limited to, ethenyl (also called “vinyl”), allyl, propenyl, crotyl, 2-isopentenyl, allenyl, butenyl, butadienyl, pentenyl, pentadienyl, 3(1,4-pentadienyl), hexenyl and hexadienyl. When the indicated number of carbon atoms is 1, then the C₁ alkenyl is double bonded to a carbon. An alkenyl group can be substituted or unsubstituted. When substituted, one or more carbon atoms may be replaced with a heteroatom such as N, O, or S.

The term alkynyl refers to a straight or branched chain hydrocarbon radical containing at least one carbon-carbon triple bond. The chain may contain an indicated number of carbon atoms. For example, “C₂-C₁₂ alkynyl” indicates that the group may have from 2 to 12 (inclusive) carbon atoms and at least one carbon-carbon triple bond. Exemplary such groups include, but are not limited to, ethynyl, propynyl and butynyl. An alkynyl group can be substituted or unsubstituted. When substituted, one or more carbon atoms may be replaced with a heteroatom such as N, O, or S.

The term “aryl” refers to cyclic aromatic carbon ring systems containing from 6 to 18 carbons. Examples of an aryl group include, but are not limited to, phenyl, naphthyl, anthracenyl, tetracenyl, and phenanthrenyl. An aryl group can be unsubstituted or substituted. When substituted, one or more carbon atoms may be replaced with a heteroatom such as N, O, or S.

The term “aralkyl” refers to an alkyl group where an H has been replaced with an aryl group. An aralkyl group may be unsubstituted or it may be substituted on the hydrocarbon chain or the aryl ring. When substituted, one or more carbon atoms may be replaced with an N, O, or S.

The term “heteroaryl” refers to mono and bicyclic aromatic groups of 4 to 10 atoms containing at least one heteroatom. Heteroatom as used in the term heteroaryl refers to oxygen, sulfur and nitrogen. A heteroaryl group can be unsubstituted or substituted.

The terms “prevent,” “preventing,” and “prevention” refer herein to the inhibition of the development or onset of a biofilm or the prevention of the recurrence, onset, or development of one or more indications or symptoms of a biofilm on a surface resulting from the administration of a composition described herein (e.g., a prophylactic or therapeutic composition), or the administration of a combination of therapies (e.g., a combination of prophylactic or therapeutic compositions).

### Polyamines

Polyamines are small organic compounds found in most cells. Polyamines putrescine (1,4-diaminobutane), spermidine (1,8-diamino-4-azaoctane) and spermine (1,12-diamino-4,9-diazaoctane) are required in micromolar to millimolar concentrations to support a wide variety of cellular functions. Depletion of polyamines can result in disruption of cellular functions and can cause cytotoxicity. For example, spermidine and spermine promote biofilm formation in some bacteria. It has therefore been surprisingly discovered that certain polyamines inhibit biofilm formation and/or disassemble existing biofilms.

This disclosure is based, at least in part, on the discovery that polyamines present in conditioned medium from mature biofilms inhibit biofilm formation and trigger the disassembly of existing biofilms. As shown in the Examples, it was discovered that the biofilm-inhibiting effect of norspermidine was specific in that a closely related polyamine, spermidine (differing only by an extra methylene group), exhibited little activity. Similarly, another polyamine, norspermine, was also active in biofilm inhibition whereas its close relative spermine (once again, having an extra methylene) was inactive. These discoveries, coupled with the results shown in the Examples, led to the development of the polyamines described herein that can inhibit biofilm formation and trigger biofilm disassembly. Polyamines discovered to be particularly suitable for use as biofilm inhibitors include polyamines comprising propylamino units and whose amino units are ionizable.

Without being bound by theory, it is believed that norspermidine acts to disrupt or inhibit biofilm by targeting the exopolysaccharide. There are several pieces of evidence, which are shown in the Examples, that support this. First, norspermidine and D-amino acids acted cooperatively in inhibiting biofilm formation, suggesting that they function by different mechanisms. Second, pellicles formed in the presence of norspermidine resembled the wispy, fragmented material produced by an exopolysaccharide mutant but not the thin, flat, featureless pellicle of a mutant blocked in amyloid-fiber production. Third, fluorescence microscopy showed that norspermidine (but not spermidine) disrupted the normal uniform pattern of staining of exopolysaccharide but had little effect on the staining pattern of the protein component of the matrix. Finally, light scattering and electron microscopy experiments revealed that norspermidine, but not spermidine, interacted with purified exopolysaccharide.

Remarkably, the biofilm-inhibiting effect of norspermidine and norspermine was not limited to B. subtilis. Both molecules inhibited the formation of submerged biofilms by S. aureus and E. coli. Indeed, the same pattern of molecules that were active or inactive in inhibiting biofilm formation by B. subtilis was observed for S. aureus and E. coli. Therefore, the polyamines described herein use a common mechanism of targeting the exopolysaccharide. Indeed, this was supported by fluorescence microscopy with S. aureus and E. coli and light scattering experiments with purified exopolysaccharide from E. coli.

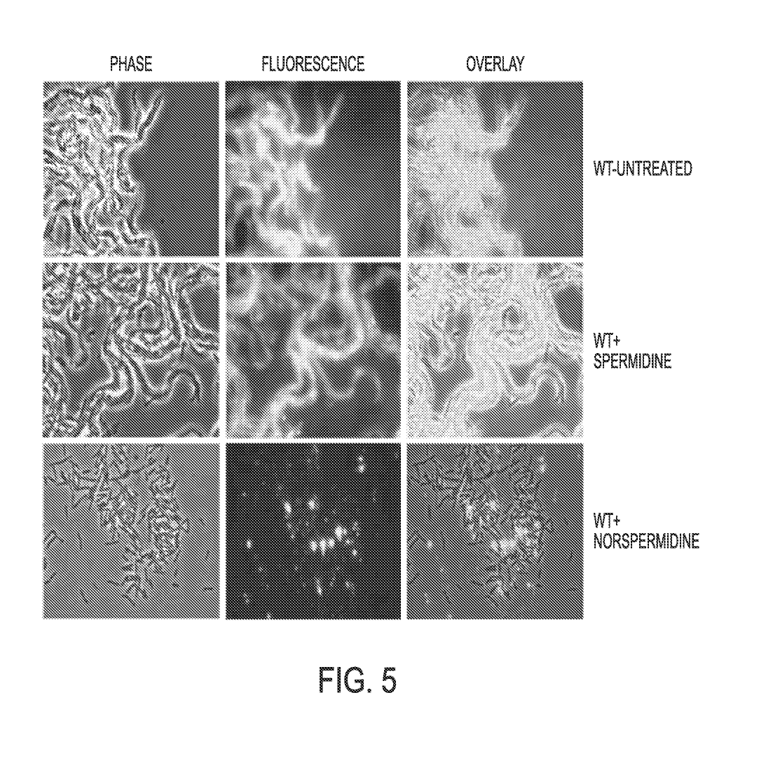

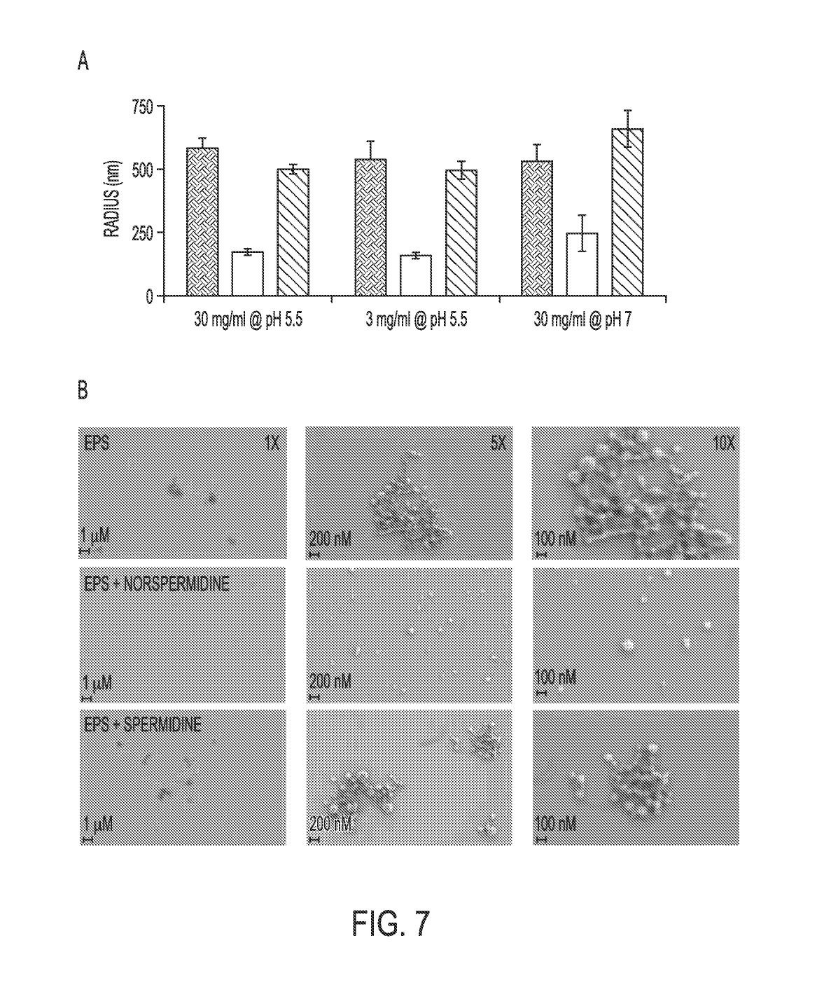

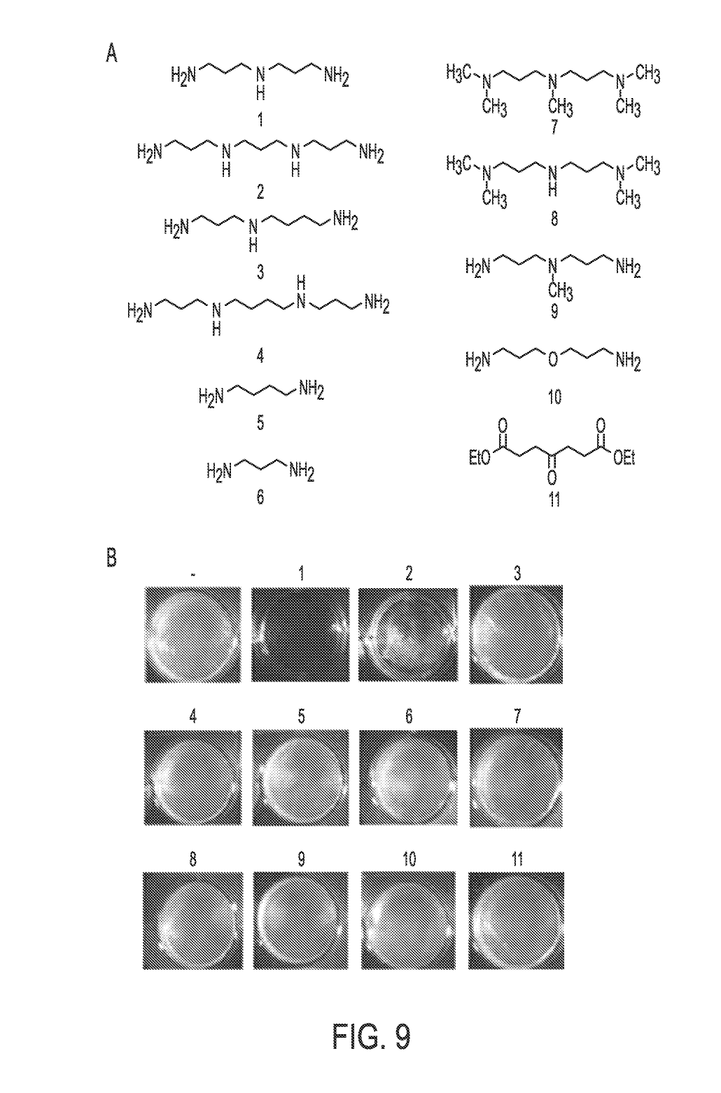

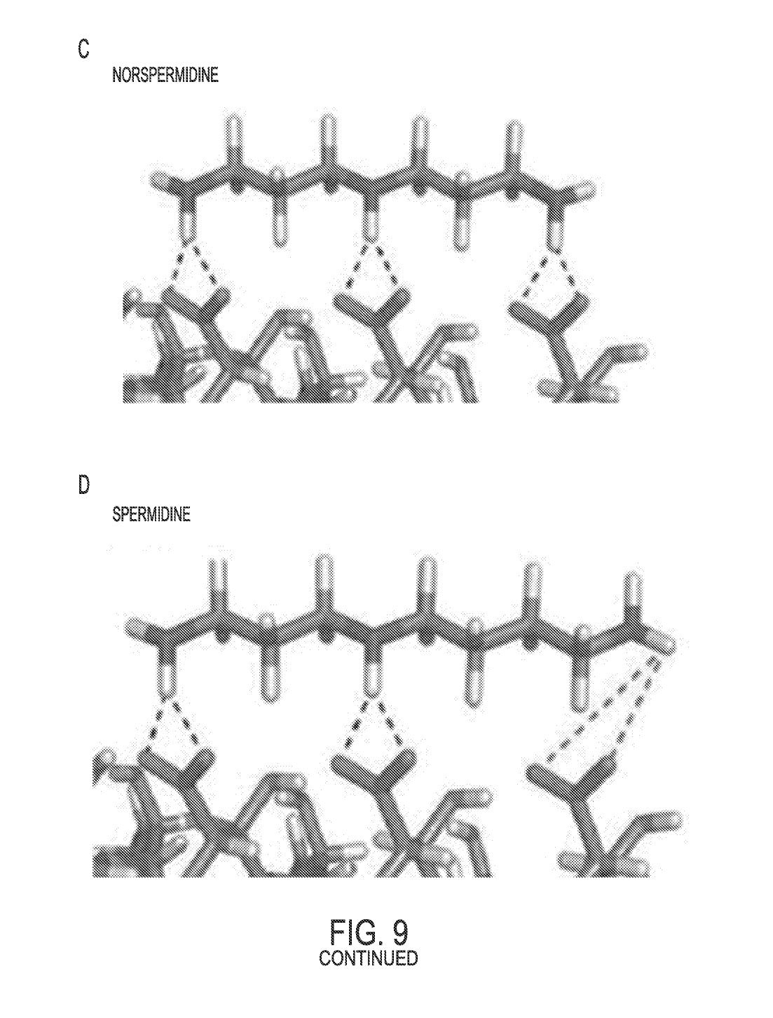

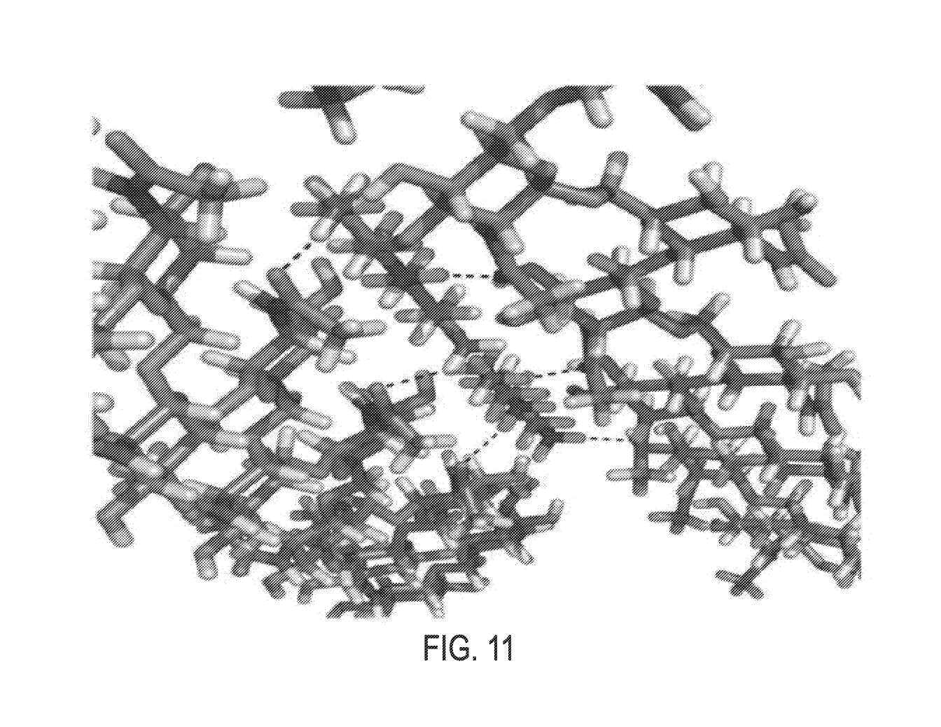

Exopolysaccharides often contain negatively charged residues (e.g. uronic acid) or neutral sugars with polar groups (e.g. poly-N-acetylglucosamine). Molecular modeling suggests that the amines in norspermidine, but not those in spermidine, are capable of interacting with such charged (FIGS. 9C and 9D) or polar groups (FIG. 11) in secondary structure of the exopolysaccharide. This interaction enhances the ability of the exopolysaccharide polymers to interact with each other or with other parts of the polymer chain. Indeed, the results of fluorescence microscopy (FIG. 5), dynamic light scattering (FIG. 7A), and scanning electron microscopy (FIG. 7B) indicate that the exopolysaccharide network collapses upon addition of norspermidine. Without being bound by theory, it is possible that exopolysaccharide polymers form an interwoven meshwork in the matrix that helps hold cells together and that condensation of the polymers in response to norspermidine weakens the meshwork and causes release of polymers.

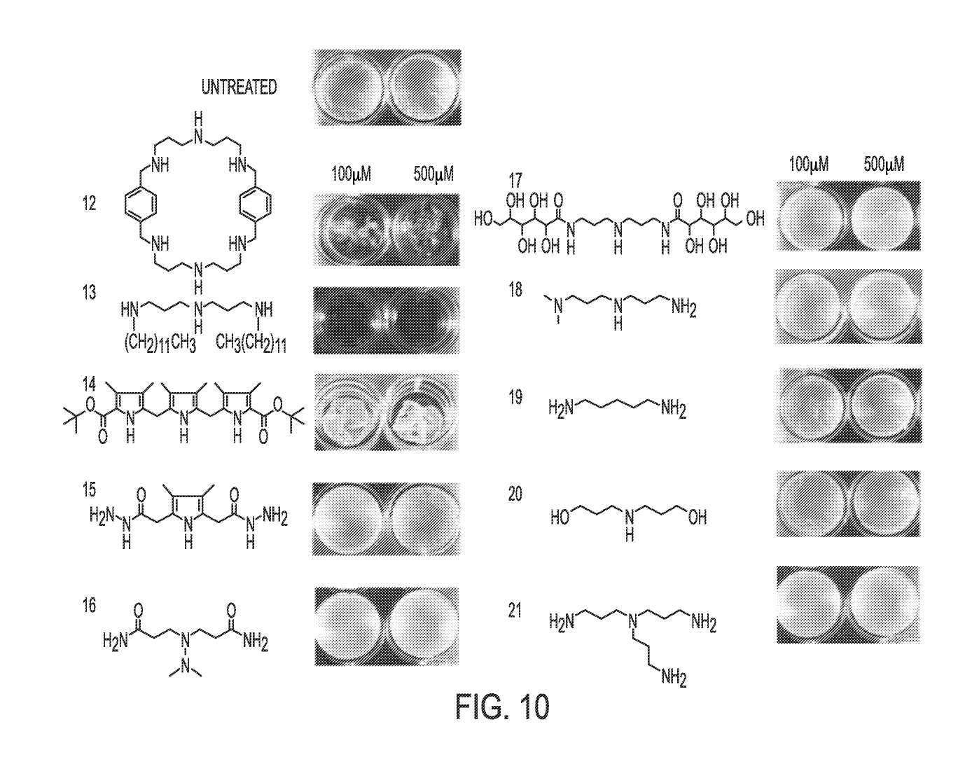

Furthermore, as seen in the examples, it was also discovered that the charge of each amine (at the neutral pH of the medium) was also important for biofilm inhibiting activity. (See Example 6). Molecules that had neutral amide bonds instead of amines separated by three methylenes (15-17) were only weakly active or inactive (FIG. 10 and Table 2). The more effective polyamines have amino groups that are ionizable. This ionizable feature of the polyamines further supports the theory that the polyamines target the exopolysaccharide.

Given the apparent versatility of norspermidine and norspermine in inhibiting biofilm formation by a variety of bacteria, the polyamines described in this application were developed to interact with the exopolysaccharides of biofilms and prevent biofilm formation by medically and industrially important microorganisms.

In some aspects, this disclosure features compositions, such as therapeutic or pharmaceutical compositions, comprising one or more polyamines. In certain embodiments, the polyamine has at least three amino groups separated by three atoms either in a straight chain or cyclic molecule. In certain embodiments, the polyamine has Formula (I),

HR₇N-(M-N(R₇)—)ₓ-M-N(R₇)—Y—N(R₇)-(M-N(R₇)—)ₓ-M-NR₇H (I)

wherein

M is —C(R₁R₂)C(R₃R₄)C(R₅R₆)—;

each R₁, R₂, R₃, R₄, R₅, and R₆ is H, C₁-C₁₂ alkyl, C₁-C₁₂ alkenyl, C₂-C₁₂ alkynyl, C₁-C₁₂ alkoxy, alkaryl, aryl, or heteroaryl, so that each R₁, R₂, R₃, R₄, R₅, and R₆ may be the same or different;

each R₇ is H, C₁-C₁₆ alkyl, C₂-C₁₆ alkenyl, C₂-C₁₆ alkynyl, aryl, heteroaryl, or C₇₋₂₂ aralkyl, so that each R₇ may be the same or different;

Y is a moiety that interrupts the polyamine chain and is C₁-C₁₂ alkyl, C₁-C₁₂ alkenyl, C₂-C₁₂ alkynyl, C₁-C₁₂ alkoxy, alkaryl, aryl, or heteroaryl, polymer block, or oligomer block; and

each x is greater than or equal to 1.

In some embodiments, the polyamines can have Formula (Ia), where some R₇ groups in Formula (I) is replaced with R₇ₐ, which is defined as H, C₁₋₈ ω-amino alkyl, C₂₋₈ ω-amino alkenyl, C₂₋₈ ω-amino alkynyl, amino alkaryl, amino aryl, N-heteroaryl. In such cases, the polyamines have Formula (Ia),

HR₇N-(M-N(R₇ₐ)—)ₓ-M-N(R₇)—Y—N(R₇ₐ)-(M-N(R₇ₐ)—)ₓ-M-NR₇H (Ia)

wherein

M is —C(R₁R₂)C(R₃R₄)C(R₅R₆)—;

each R₁, R₂, R₃, R₄, R₅, and R₆ is H, C₁-C₁₂ alkyl, C₁-C₁₂ alkenyl, C₂-C₁₂ alkynyl, C₁-C₁₂ alkoxy, alkaryl, aryl, or heteroaryl, so that each R₁, R₂, R₃, R₄, R₅, and R₆ may be the same or different;

each R₇ is H, C₁-C₁₆ alkyl, C₂-C₁₆ alkenyl, C₂-C₁₆ alkynyl, aryl, heteroaryl, or C₇₋₂₂ aralkyl, so that each R₇ may be the same or different;

each R₇ₐ is H, C₁₋₈ ω-amino alkyl, C₂₋₈ ω-amino alkenyl, C₂₋₈ ω-amino alkynyl, amino alkaryl, amino aryl, N-heteroaryl, so that each R₇ may be the same or different;

Y is a moiety that interrupts the polyamine chain and is C₁-C₁₂ alkyl, C₁-C₁₂ alkenyl, C₂-C₁₂ alkynyl, C₁-C₁₂ alkoxy, alkaryl, aryl, or heteroaryl, polymer block, or oligomer block; and

each x is greater than or equal to 1.

Compounds of Formulae (I) may be acyclic or cyclic. If a compound of Formulae (I) is cyclic, the terminal NHR₁ groups of Formula (I) are NR₁ and form a ring where each NR₁ covalently bonds to one M group, thereby forming a ring.

In certain embodiments, the polymer or oligomer blocks of Y can comprise one or more of carbonyl, epoxy, ester, carboxyl, amine, amide, imine, imide, or glycol.

In some embodiments, the polyamine has Formula (II),

Hₐ-[R₇N-(L-N(R₈)—)ₓ-L-NH—P_{b}]_{c} (II),

wherein

each P is R₇ or Q or L;

each L is M or —C(R₁R₂)—X—C(R₅R₆)— or

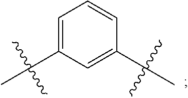 [SMILES: *c1cccc(*)c1]

each M is —C(R₁R₂)C(R₃R₄)C(R₅R₆)—;

each R₁, R₂, R₃, R₄, R₅, and R₆ is H, C₁-C₁₂ alkyl, C₁-C₁₂ alkenyl, C₂-C₁₂ alkynyl, C₁-C₁₂ alkoxy, alkaryl, aryl, or heteroaryl, so that each R₁, R₂, R₃, R₄, R₅, and R₆ may be the same or different;

each R₇ is H, C₁-C₁₆ alkyl, C₂-C₁₆ alkenyl, C₂-C₁₆ alkynyl, aryl, heteroaryl, or C₇₋₂₂ aralkyl which may be substituted on the aryl ring, or R₈ so that each R₇ may be the same or different, wherein R₇ is R₈ when the polyamine is cyclic;

each R₈ is H, C₁₋₈ ω-amino alkyl, C₂₋₈ ω-amino alkenyl, C₂₋₈ ω-amino alkynyl, amino alkaryl, amino aryl, N-heteroaryl;

each X is —NH—, —O—, —N(R₈)—, or S;

each Q is C₁₋₃₀ alkyl, C₂₋₃₀ alkenyl, C₂₋₃₀ alkynyl, alkaryl, aryl, heteroaryl, which may be substituted and where the alkyl, alkenyl, alkynyl, and alkaryl may be interrupted by one or more heteroatoms such as N, O, or S;

a is 0 or 1, wherein when a is 0 the polyamine is cyclic and when a is 1 the polyamine is linear or branched;

each b is 0 or 1;

c is greater than or equal to 1; and

each x is greater than or equal to 1.

In some embodiments of Formula (II), a is 1 and the polyamine is linear or branched. In certain embodiments of Formula (II), a is 1, b is 1, c is 1, L is M, P is R₇, R₇ and R₈ are hydrogen, and the polyamine has Formula (IIa),

H₂N-(M-NH)ₓ-M-NH₂ (IIa).

In certain embodiments a is 0 and compounds of Formula (II) may be cyclic. In some embodiments, a is 0, b is 1, P is Q, and the ring is formed by Q bonding to the terminal NR₇ group. In some embodiments, a is 0, b is 1, P is L, L is M, R₇ and R₈ are hydrogen, the terminal NR_{S} group (where R₇ is hydrogen) bonds to the terminal L group (which is M), and the cyclic polyamine has Formula (IIb),

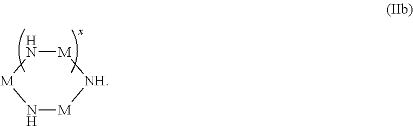

Compounds of Formulae (I), (Ia), (II), and (IIa) may be linear or branched.

In some embodiments of compounds of Formulae (I), (Ia), (II), (IIa), and (IIb), x may be 1. In some embodiments of compounds of Formulae (I), (Ia), (II), (IIa), and (IIb), x may be 2. In other embodiments of compounds of the above Formulae (I), (Ia), (II), (IIa), and (IIb), x may be 3. In still other embodiments of compounds of the above Formulae (I), (Ia), (II), (IIa), and (IIb), x may be 4. In still other embodiments of compounds of the above Formulae (I), (Ia), (II), (IIa), and (IIb), x may be 5. In further embodiments of compounds of the above Formulae (I), (Ia), (II), (IIa), and (IIb), x may be greater than 5.

In some embodiments, M is —CH₂CH₂CH₂—. In some embodiments, R₇ is —(CH₂)₁₁CH₃. In other embodiments, R₇ is hydrogen. In some embodiments, R₈ is hydrogen. In other embodiments, R₈ is —(CH₂)₃NH₂.

In some embodiments, at least one of R₁, R₂, R₃, R₄, R₅, or R₆ is C₁alkenyl and the other R group attached to the same carbon atom does not exist. For example, if R₁ is C₁-alkenyl, then R₂ does not exist.

In some embodiments, L is

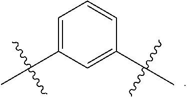 [SMILES: *c1cccc(*)c1]

Compound i in Table 1 exemplifies such an embodiment.

In some embodiments, the composition comprises norspermidine (also known as N-(3-aminopropyl)propane-1,3-diamine), norspermine (N′-[3-(3-aminopropylamino)propyl]propane-1,3-diamine), 1,5,9-triazacyclododecane, or a combination thereof. In further embodiments, the composition comprises two or more of norspermidine, norspermine, and 1,5,9-triazacyclododecane. In certain embodiments, the polyamine has Formulae (I), (Ia), (II), (IIa), or (IIb). In other embodiments, the polyamine is a compound in Table 1.

TABLE 1

| Com- | |
|---|---|
| pound | Formula |
| a | 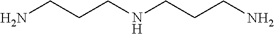 Norspermidine (also known as N-(3-aminopropyl)propane- 1,3-diamine) |
| b | 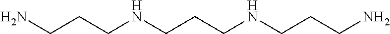 [SMILES: NCCCNCCCNCCCN] Norspermine |
| c | 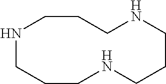 [SMILES: C1CNCCCNCCCNC1] 1,5,9-triazacyclododecane |
| d | 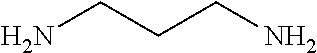 1,3-diaminopropane |
| e | 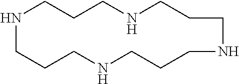 [SMILES: C1CNCCCNCCCNCCCNC1] 1,5,9,13-tetraazacyclohexadecane |
| f | 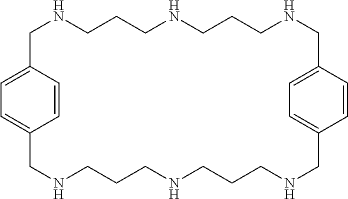 [SMILES: c1cc2ccc1CNCCCNCCCNCc1ccc(cc1)CNCCCNCCCNC2] 3,7,11,18,22,26-Hexaazatricyclo[26.2.2.213,16]tetratriaconta- 13,15,28,30,31,33-hexaene |
| g | 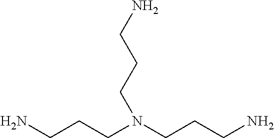 [SMILES: NCCCN(CCCN)CCCN] N¹,N¹-bis(3-aminopropyl)propane-1,3-diamine |
| h | 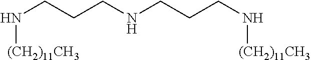 N1-dodecyl-N3-(3-(dodecylamino)propyl)propane-1,3-diamine |
| i | 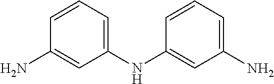 [SMILES: Nc1cccc(Nc2cccc(N)c2)c1] N,N-Di(3-aminophenyl)amine |

In some embodiments, compositions of the present disclosure include a compound from Table 1, or a combination of one or more compounds from Table 1. In some embodiments, the composition comprises norspermidine (also known as N-(3-aminopropyl)propane-1,3-diamine), norspermine (N′-[3-(3-aminopropylamino)propyl]propane-1,3-diamine), 1,5,9-triazacyclododecane, or a combination thereof. In further embodiments, the composition comprises two or more of norspermidine, norspermine, and 1,5,9-triazacyclododecane.

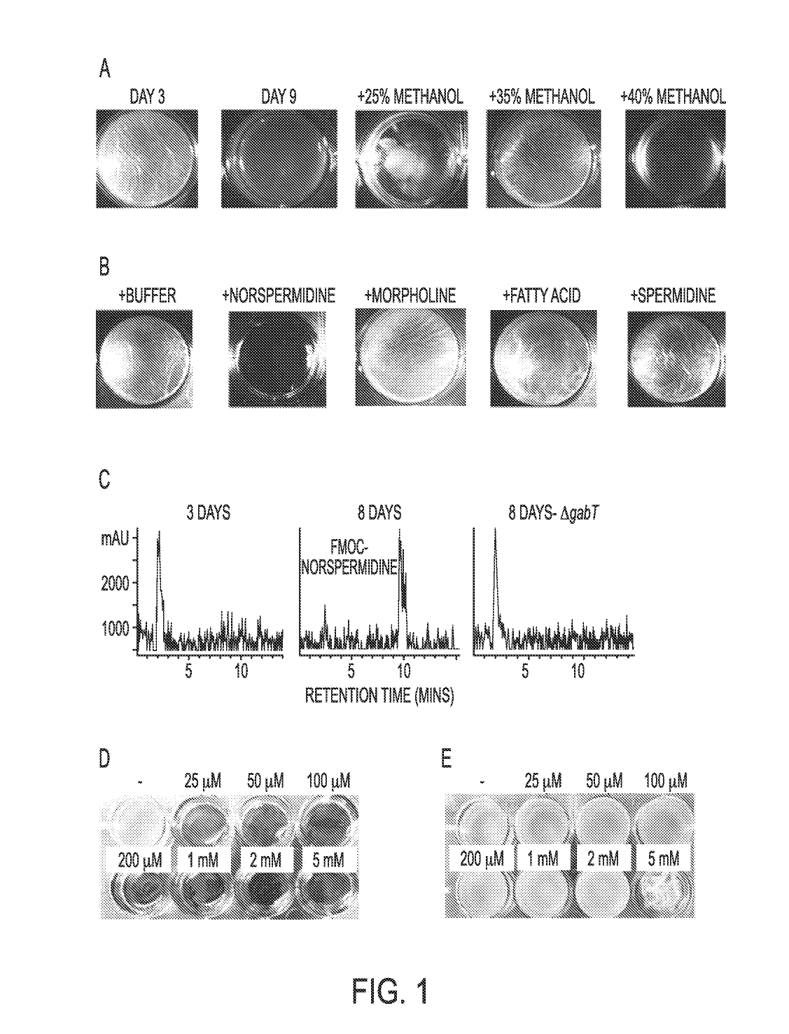

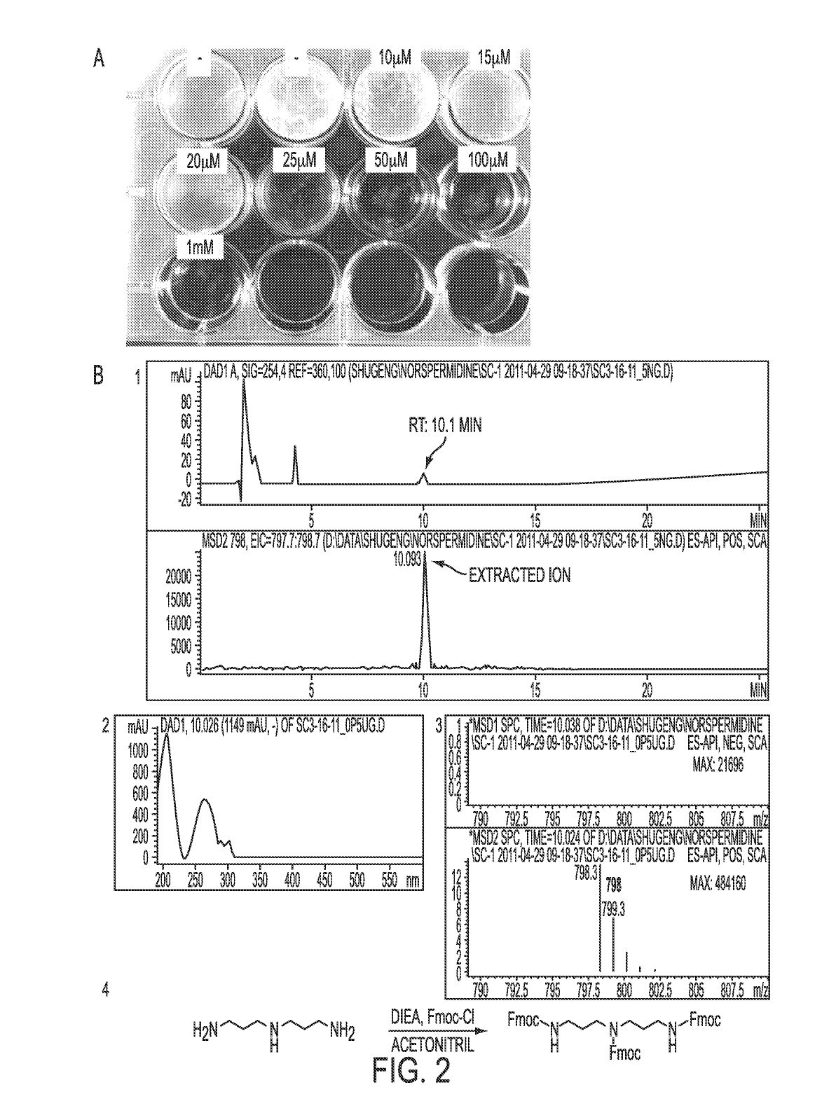

In one or more embodiments, polyamines can inhibit biofilm formation in cell populations, and in particular, in biofilm-forming bacteria. By way of example, polyamines such as norspermidine and norspermine significantly retard the formation of biofilm in bacterial colonies such as Staphylococcus aureus, Bacillus subtilis, and Pseudomonas aeruginosa. See, e.g., FIGS. 1, 2, 7 and 9, and Examples 1, 2, 7, and 9. Polyamines have been demonstrated to reduce biofilm-forming activity by measuring the OD600 of cells that adhere to the surface as a measure of biofilm formation.

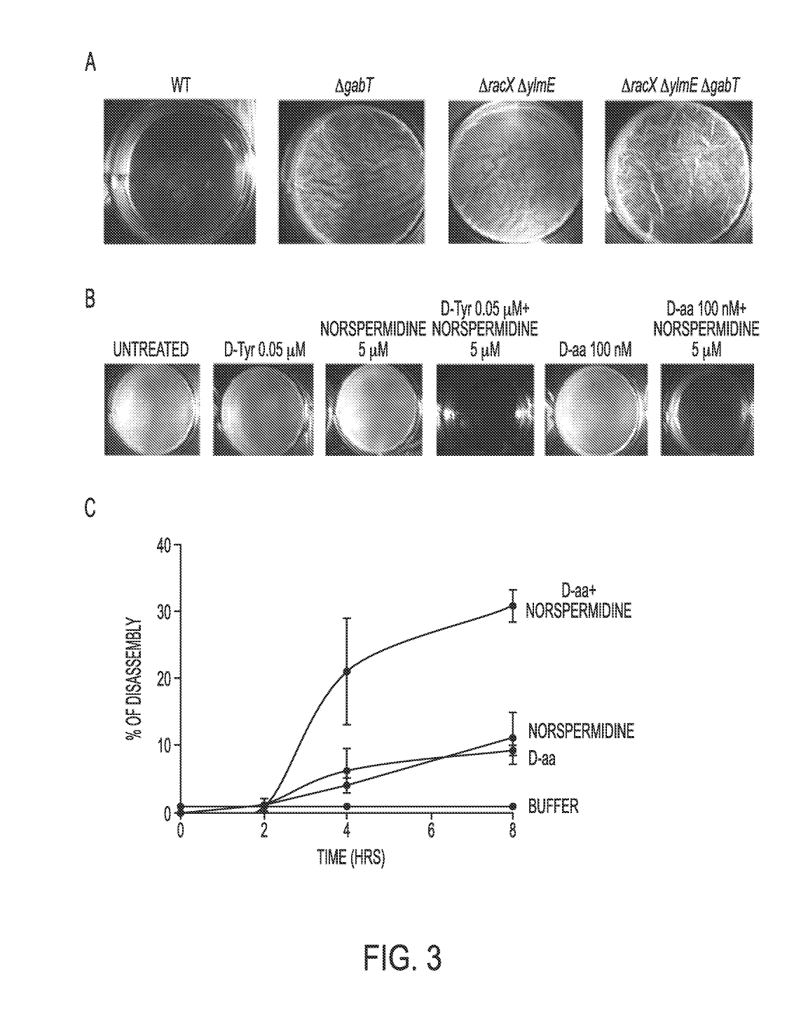

In one or more embodiments, polyamines can disrupt established biofilms. Even after bacteria have established a biofilm, contact of the biofilm with a solution containing a polyamine results in the disruption and disassembly of the pellicle. By way of example, polyamines such as norspermine can disrupt pellicles formed by bacterial colonies such as Bacillus subtilis. See, e.g., FIG. 3 and Example 3. Polyamines have been demonstrated to reduce biofilm-forming activity by measuring the OD600 of cells in free medium and compared to the OD600 in the residual pellicle.

A polyamine can be effective at a concentration of 0.1 nM to 100 μM, e.g., 1 nM to 10 μM, 5 nM to 5 μM, or 10 nM to 1 μM. In other embodiments, a polyamine can be effective at a concentration of about 0.1 nM to about 100 μM, e.g., about 1 nM to about 10 μM, about 5 nM to about 5 μM, or about 10 nM to about 1 μM.

Exemplary polyamines found to be particularly effective in inhibiting or treating biofilm formation include norspermidine, norspermine, 1,5,9-triazacyclododecane, and other compounds in Table 1. Norspermidine, norspermine, or 1,5,9-triazacyclododecane can be used, for example, at concentrations of less than 1 mM, or less than 100 μM or less than 10 μM, or at a concentration of 0.1 nM to 100 μM, e.g., 1 nM to 10 μM, 5 nM to 5 μM, or 10 nM to 1 μM.

### Polyamines and Amino Acids

It has been surprisingly discovered that a polyamine and a D-amino acid can act synergistically to inhibit biofilm formation or trigger biofilm disassembly. It has been discovered that polyamines and D-amino acids inhibit biofilm formation and trigger biofilm disassembly by different mechanisms. Thus, in one or more embodiments, a polyamine can be co administered with an amino acid, and in particular with a D-amino acid, to inhibit biofilm formation or trigger biofilm disassembly. The different mechanisms by which the polyamines and D-amino acids work result in synergism between the polyamine and D-amino acid and, in some embodiments, lower amounts of polyamines and D-amino acids are used to inhibit biofilm formation and/or trigger biofilm disassembly.

Standard amino acids can exist in either of two optical isomers, called L- or D-amino acids, which are mirror images of each other. While L-amino acids represent the vast majority of amino acids found in proteins, D-amino acids are components of the peptidoglycan cell walls of bacteria. The D-amino acids described herein are capable of penetrating biofilms on living and non-living surfaces, of preventing the adhesion of bacteria to surfaces and any further build-up of the biofilm, of detaching such biofilm and/or inhibiting the further growth of the biofilm-forming micro-organisms in the biological matrix, or of killing such micro-organisms.

D-amino acids are known in the art and can be prepared using known techniques. Exemplary methods include, e.g., those described in U.S. Publ. No. 20090203091. D-amino acids are also commercially available (e.g., from Sigma Chemicals, St. Louis, Mo.).

Any D-amino acid can be used in the methods described herein, including without limitation D-alanine, D-cysteine, D-aspartic acid, D-glutamic acid, D-phenylalanine, D-histidine, D-isoleucine, D-lysine, D-leucine, D-methionine, D-asparagine, D-proline, D-glutamine, D-arginine, D-serine, D-threonine, D-valine, D-tryptophan, or D-tyrosine. A D-amino acid can be used alone or in combination with other D-amino acids. In exemplary methods, 2, 3, 4, 5, 6, or more D-amino acids are used in combination. Preferably, D-tyrosine, D-leucine, D-methionine, or D-tryptophan, either alone or in combination, are used in the methods described herein. In other preferred embodiments, D-tyrosine, D-proline and D-phenylalanine, either alone or in combination, are used in the methods described herein.

A D-amino acid combined with a polyamine can be administered at a concentration of 0.1 nM to 100 μM, e.g., 1 nM to 10 μM, 5 nM to 5 μM, or 10 nM to 1 μM. In other embodiments, a D-amino acid can be administered at a concentration of about 0.1 nM to about 100 μM, e.g., about 1 nM to about 10 μM, about 5 nM to about 5 μM, or about 10 nM to about 1 μM.

An exemplary D-amino acid found to be particularly effective in inhibiting or treating biofilm formation when combined with a polyamine includes D-tyrosine. In some embodiments, D-tyrosine can be used, for example, as concentrations of less than 1 mM, or less than 100 μM or less than 10 μM, or at a concentration of 0.1 nM to 100 μM, e.g., 1 nM to 10 μM, 5 nM to 5 μM, or 10 nM to 1 μM.

In other embodiments, D-tyrosine is used in combination with one or more of D-proline and D-phenylalanine. In some embodiments, D-tyrosine is used in combination with one or more of D-leucine, D-tryptophan, and D-methionine. The combinations of D-tyrosine with one or more of D-proline, D-phenylalanine, D-leucine, D-tryptophan, and D-methionine can be synergistic and can be effective in inhibiting or treating biofilm formation at total D-amino acid concentrations of 10 μM or less, e.g., about 1 nM to about 10 μM, about 5 nM to about 5 μM, or about 10 nM to about 1 μM, or at a concentration of 0.1 nM to 100 μM, e.g., 1 nM to 10 μM, 5 nM to 5 μM, or 10 nM to 1 μM.

In some embodiments, the combinations of polyamines and D-amino acids are equimolar. In some embodiments, the combinations of D-amino acids are equimolar. In other embodiments, the combinations of D-amino acids are not in equimolar amounts.

In some embodiments, the composition is essentially free of L-amino acids. For example, the composition comprises less than about 30%, less than about 20%, less than about 10%, less than about 5%, less than about 1%, less than about 0.5%, less than about 0.25%, less than about 0.1%, less than about 0.05%, less than about 0.025%, less than about 0.01%, less than about 0.005%, less than about 0.0025%, less than about 0.001%, or less, of L-amino acids. In other embodiments, the composition comprises less than 30%, less than 20%, less than 10%, less than 5%, less than 1%, less than 0.5%, less than 0.25%, less than 0.1%, less than 0.05%, less than 0.025%, less than 0.01%, less than 0.005%, less than 0.0025%, less than 0.001% of L-amino acids. In preferred embodiments, the percentage of L-amino acid is relative to the corresponding D-amino acid. By way of example, a racemic mixture of L-amino acid and D-amino acid contains 50% L-amino acid.

In some embodiments, the composition is essentially free of detergent. For example, the composition comprises, less than about 30 wt %, less than about 20 wt %, less than about 10 wt %, less than about 5 wt %, less than about 1 wt %, less than about 0.5 wt %, less than about 0.25 wt %, less than about 0.1 wt %, less than about 0.05 wt %, less than about 0.025 wt %, less than about 0.01 wt %, less than about 0.005 wt %, less than about 0.0025 wt %, less than about 0.001 wt %, or less, of a detergent. In other embodiments, the composition comprises, relative to the overall composition, less than about 30 wt %, less than 20 wt %, less than 10 wt %, less than 5 wt %, less than 1 wt %, less than 0.5 wt %, less than 0.25 wt %, less than 0.1 wt %, less than 0.05 wt %, less than 0.025 wt %, less than 0.01 wt %, less than 0.005 wt %, less than 0.0025 wt %, less than 0.001 wt % of a detergent. Many times in formulations containing detergents, e.g., surfactants, the surfactant will interact with the active agent, which could greatly affect the agent's efficacy. In some embodiments, it can be necessary to screen agents effectiveness relative to anionic surfactants, cationic surfactants, non-ionic surfactants and zwitter ionic surfactants as a screening to determine if the presence of the surfactant type alters the efficacy. Reducing or eliminating detergents, can increase the efficacy of the compositions and/or reduce formulation complications.

In other embodiments, the composition is essentially free of both detergent and L-amino acids.

### Biofilms

Most bacteria can form complex, matrix-containing multicellular communities known as biofilms (O'Toole et al., Annu Rev. Microbiol. 54:49 (2000); López et al., FEMS Microbiol. Rev. 33:152 (2009); Karatan et al., Microbiol. Mol. Biol. Rev. 73:310 (2009)). Biofilm-associated bacteria are protected from environmental insults, such as antibiotics (Bryers, Biotechnol. Bioeng. 100:1 (2008)). However, as biofilms age, nutrients become limiting, waste products accumulate, and it is advantageous for the biofilm-associated bacteria to return to a planktonic existence (Karatan et al., Microbiol. Mol. Biol. Rev. 73:310 (2009)). Thus, biofilms have a finite lifetime, characterized by eventual disassembly.

Gram-negative bacteria, Gram-positive bacteria, and mycobacteria, in addition to other unicellular organisms, can produce biofilms. Bacterial biofilms are surface-attached communities of cells that are encased within an extracellular polysaccharide matrix produced by the colonizing cells. Biofilm development occurs by a series of programmed steps, which include initial attachment to a surface, formation of three-dimensional microcolonies, and the subsequent development of a mature biofilm. The more deeply a cell is located within a biofilm (such as, the closer the cell is to the solid surface to which the biofilm is attached to, thus being more shielded and protected by the bulk of the biofilm matrix), the more metabolically inactive the cells are. The consequences of this physiologic variation and gradient create a collection of bacterial communities where there is an efficient system established whereby microorganisms have diverse functional traits. A biofilm also is made up of various and diverse non-cellular components and can include, but are not limited to carbohydrates (simple and complex), lipids, proteins (including polypeptides), and lipid complexes of sugars and proteins (lipopolysaccharides and lipoproteins). A biofilm may include an integrated community of two or more bacteria species (polymicrobic biofilms), or predominantly one specific bacterium.

In one or more embodiments, a polyamine can be coadministered with an amino acid, and in particular with a D-amino acid. As shown in the Examples, a polyamine and a D amino acid can act synergistically to inhibit biofilm formation or trigger biofilm disassembly. Standard amino acids can exist in either of two optical isomers, called L- or D-amino acids, which are mirror images of each other. While L-amino acids represent the vast majority of amino acids found in proteins, D-amino acids are components of the peptidoglycan cell walls of bacteria. The D-amino acids described herein are capable of penetrating biofilms on living and non-living surfaces, of preventing the adhesion of bacteria to surfaces and any further build-up of the biofilm, of detaching such biofilm and/or inhibiting the further growth of the biofilm-forming micro-organisms in the biological matrix, or of killing such micro-organisms.

D-amino acids are known in the art and can be prepared using known techniques. Exemplary methods include, e.g., those described in U.S. Publ. No. 20090203091. D-amino acids are also commercially available (e.g., from Sigma Chemicals, St. Louis, Mo.).

Any D-amino acid can be used in the methods described herein, including without limitation D-alanine, D-cysteine, D-aspartic acid, D-glutamic acid, D-phenylalanine, D-histidine, D-isoleucine, D-lysine, D-leucine, D-methionine, D-asparagine, D-proline, D-glutamine, D-arginine, D-serine, D-threonine, D-valine, D-tryptophan, or D-tyrosine. A D-amino acid can be used alone or in combination with other D-amino acids. In exemplary methods, 2, 3, 4, 5, 6, or more D-amino acids are used in combination. Preferably, D-tyrosine, D-leucine, D-methionine, or D-tryptophan, either alone or in combination, are used in the methods described herein. In other preferred embodiments, D-tyrosine, D-proline and D-phenylalanine, either alone or in combination, are used in the methods described herein.

A D-amino acid combined with a polyamine can be administered at a concentration of 0.1 nM to 100 μM, e.g., 1 nM to 10 μM, 5 nM to 5 μM, or 10 nM to 1 μM. In other embodiments, a D-amino acid can be administered at a concentration of about 0.1 nM to about 100 μM, e.g., about 1 nM to about 10 μM, about 5 nM to about 5 μM, or about 10 nM to about 1 μM.

An exemplary D-amino acid found to be particularly effective in inhibiting or treating biofilm formation when combined with a polyamine includes D-tyrosine. In some embodiments, D-tyrosine can be used, for example, as concentrations of less than 1 mM, or less than 100 μM or less than 10 μM, or at a concentration of 0.1 nM to 100 μM, e.g., 1 nM to 10 μM, 5 nM to 5 μM, or 10 nM to 1 μM.

In other embodiments, D-tyrosine is used in combination with one or more of D-proline and D-phenylalanine. In some embodiments, D-tyrosine is used in combination with one or more of D-leucine, D-tryptophan, and D-methionine. The combinations of D-tyrosine with one or more of D-proline, D-phenylalanine, D-leucine, D-tryptophan, and D-methionine can be synergistic and can be effective in inhibiting or treating biofilm formation at total D-amino acid concentrations of 10 μM or less, e.g., about 1 nM to about 10 μM, about 5 nM to about 5 μM, or about 10 nM to about 1 μM, or at a concentration of 0.1 nM to 100 μM, e.g., 1 nM to 10 μM, 5 nM to 5 μM, or 10 nM to 1 μM.

In some embodiments, the combinations of polyamines and D-amino acids are equimolar. In some embodiments, the combinations of D-amino acids are equimolar. In other embodiments, the combinations of D-amino acids are not in equimolar amounts.

In some embodiments, the composition is essentially free of L-amino acids. For example, the composition comprises less than about 30%, less than about 20%, less than about 10%, less than about 5%, less than about 1%, less than about 0.5%, less than about 0.25%, less than about 0.1%, less than about 0.05%, less than about 0.025%, less than about 0.01%, less than about 0.005%, less than about 0.0025%, less than about 0.001%, or less, of L-amino acids. In other embodiments, the composition comprises less than 30%, less than 20%, less than 10%, less than 5%, less than 1%, less than 0.5%, less than 0.25%, less than 0.1%, less than 0.05%, less than 0.025%, less than 0.01%, less than 0.005%, less than 0.0025%, less than 0.001% of L-amino acids. In preferred embodiments, the percentage of L-amino acid is relative to the corresponding D-amino acid. By way of example, a racemic mixture of L-amino acid and D-amino acid contains 50% L-amino acid.

In some embodiments, the composition is essentially free of detergent. For example, the composition comprises, less than about 30 wt %, less than about 20 wt %, less than about 10 wt %, less than about 5 wt %, less than about 1 wt %, less than about 0.5 wt %, less than about 0.25 wt %, less than about 0.1 wt %, less than about 0.05 wt %, less than about 0.025 wt %, less than about 0.01 wt %, less than about 0.005 wt %, less than about 0.0025 wt %, less than about 0.001 wt %, or less, of a detergent. In other embodiments, the composition comprises, relative to the overall composition, less than about 30 wt %, less than 20 wt %, less than 10 wt %, less than 5 wt %, less than 1 wt %, less than 0.5 wt %, less than 0.25 wt %, less than 0.1 wt %, less than 0.05 wt %, less than 0.025 wt %, less than 0.01 wt %, less than 0.005 wt %, less than 0.0025 wt %, less than 0.001 wt % of a detergent. Many times in formulations containing detergents, e.g., surfactants, the surfactant will interact with the active agent, which could greatly affect the agent's efficacy. In some embodiments, it can be necessary to screen agents effectiveness relative to anionic surfactants, cationic surfactants, non-ionic surfactants and zwitter ionic surfactants as a screening to determine if the presence of the surfactant type alters the efficacy. Reducing or eliminating detergents, can increase the efficacy of the compositions and/or reduce formulation complications.

### Biofilms

Most bacteria can form complex, matrix-containing multicellular communities known as biofilms (O'Toole et al., Annu Rev. Microbiol. 54:49 (2000); López et al., FEMS Microbiol. Rev. 33:152 (2009); Karatan et al., Microbiol. Mol. Biol. Rev. 73:310 (2009)). Biofilm-associated bacteria are protected from environmental insults, such as antibiotics (Bryers, Biotechnol. Bioeng. 100:1 (2008)). However, as biofilms age, nutrients become limiting, waste products accumulate, and it is advantageous for the biofilm-associated bacteria to return to a planktonic existence (Karatan et al., Microbiol. Mol. Biol. Rev. 73:310 (2009)). Thus, biofilms have a finite lifetime, characterized by eventual disassembly.

Gram-negative bacteria and Gram-positive bacteria, in addition to other unicellular organisms, can produce biofilms. Bacterial biofilms are surface-attached communities of cells that are encased within an extracellular polysaccharide matrix produced by the colonizing cells. Biofilm development occurs by a series of programmed steps, which include initial attachment to a surface, formation of three-dimensional microcolonies, and the subsequent development of a mature biofilm. The more deeply a cell is located within a biofilm (such as, the closer the cell is to the solid surface to which the biofilm is attached to, thus being more shielded and protected by the bulk of the biofilm matrix), the more metabolically inactive the cells are. The consequences of this physiologic variation and gradient create a collection of bacterial communities where there is an efficient system established whereby microorganisms have diverse functional traits. A biofilm also is made up of various and diverse non-cellular components and can include, but are not limited to carbohydrates (simple and complex), lipids, proteins (including polypeptides), and lipid complexes of sugars and proteins (lipopolysaccharides and lipoproteins). A biofilm may include an integrated community of two or more bacteria species (polymicrobic biofilms), or predominantly one specific bacterium.

The biofilm can allow bacteria to exist in a dormant state for a certain amount of time until suitable growth conditions arise thus offering the microorganism a selective advantage to ensure its survival. However, this selection can pose serious threats to human health in that biofilms have been observed to be involved in about 65% of human bacterial infections (Smith, Adv. Drug Deliv. Rev. 57:1539-1550 (2005); Hall-Stoodley et al., Nat. Rev. Microbiol. 2:95-108 (2004)).

Biofilms can also affect a wide variety of biological, medical, commercial, industrial, and processing operations, as described herein. In industrial settings, biofilms can adhere to surfaces, such as pipes and filters. Biofilms are problematic in industrial settings because they cause biocorrosion and biofouling in industrial systems, such as heat exchangers, oil pipelines, water systems, filters, and the like (Coetser et al., (2005) Crit. Rev. Micro. 31: 212-32). Thus, biofilms can inhibit fluid flow-through in pipes, clog water and other fluid systems, as well as serve as reservoirs for pathogenic bacteria, protozoa, and fungi. As such, industrial biofilms are an important cause of economic inefficiency in industrial processing systems. Further, different species of biofilm-producing bacteria may coexist within such system. Thus, there exists in such systems the potential of biofilm formation due to multiple species.

The methods and materials described herein can prevent or reduce biofilm formation associated with a wide variety of commercial, industrial, and processing operations, such as those found in water handling/processing industries. In some instances, a polyamine, or a combination of a polyamine and D-amino acid, can be applied to a biofilm found on such surfaces. In other instances, a polyamine, or a combination of a polyamine and D-amino acid, can be utilized to prevent biofilm-forming bacteria from adhering to surfaces. For example, the surface can be a surface on industrial equipment (such as equipment located in Good Manufacturing Practice (GMP) facilities, food processing plants, photo processing venues, and the like), the surfaces of plumbing systems, or the surfaces bodies of water (such as lakes, swimming pools, oceans, and the like).

The surfaces can be coated, sprayed, or impregnated with a polyamine, or a combination of a polyamine and D-amino acid, prior to use to prevent the formation of bacterial biofilms. Specific nonlimiting examples of such surfaces include plumbing, tubing, and support components involved with water condensate collections, sewerage discharges, paper pulping operations, re-circulating water systems (such as air conditioning systems, a cooling tower, and the like), and, in water bearing, handling, processing, and collection systems. Adding a polyamine, or a combination of a polyamine and D-amino acid, can treat, prevent or reduce formation of biofilms on the surface of the water or on the surface of pipes or plumbing of water-handling systems, or other surfaces involved in the collection and/or operation systems that the water contacts.

As described herein, biofilms can also affect a wide variety of biological, medical, and processing operations.

### Biofilm-Forming Bacteria

The methods described herein can be used to prevent or delay the formation of, and/or treat, biofilms. In exemplary methods, the biofilms are formed by biofilm-forming bacteria. The bacteria can be a gram negative bacterial species or a gram positive bacterial species. Nonlimiting examples of such bacteria include a member of the genus Actinobacillus (such as Actinobacillus actinomycetemcomitans), a member of the genus Acinetobacter (such as Acinetobacter baumannii), a member of the genus Aeromonas, a member of the genus Bordetella (such as Bordetella pertussis, Bordetella bronchiseptica, or Bordetella parapertussis), a member of the genus Brevibacillus, a member of the genus Brucella, a member of the genus Bacteroides (such as Bacteroides fragilis), a member of the genus Burkholderia (such as Burkholderia cepacia or Burkholderia pseudomallei), a member of the genus Borelia (such as Borelia burgdorferi), a member of the genus Bacillus (such as Bacillus anthracis or Bacillus subtilis), a member of the genus Campylobacter (such as Campylobacter jejuni), a member of the genus Capnocytophaga, a member of the genus Cardiobacterium (such as Cardiobacterium hominis), a member of the genus Citrobacter, a member of the genus Clostridium (such as Clostridium tetani or Clostridium difficile), a member of the genus Chlamydia (such as Chlamydia trachomatis, Chlamydia pneumoniae, or Chlamydia psiffaci), a member of the genus Eikenella (such as Eikenella corrodens), a member of the genus Enterobacter, a member of the genus Escherichia (such as Escherichia coli), a member of the genus Francisella (such as Francisella tularensis), a member of the genus Fusobacterium, a member of the genus Flavobacterium, a member of the genus Haemophilus (such as Haemophilus ducreyi or Haemophilus influenzae), a member of the genus Helicobacter (such as Helicobacter pylori), a member of the genus Kingella (such as Kingella kingae), a member of the genus Klebsiella (such as Klebsiella pneumoniae), a member of the genus Legionella (such as Legionella pneumophila), a member of the genus Listeria (such as Listeria monocytogenes), a member of the genus Leptospirae, a member of the genus Moraxella (such as Moraxella catarrhalis), a member of the genus Morganella, a member of the genus Mycoplasma (such as Mycoplasma hominis or Mycoplasma pneumoniae), a member of the genus Mycobacterium (such as Mycobacterium tuberculosis or Mycobacterium leprae), a member of the genus Neisseria (such as Neisseria gonorrhoeae or Neisseria meningitidis), a member of the genus Pasteurella (such as Pasteurella multocida), a member of the genus Proteus (such as Proteus vulgaris or Proteus mirablis), a member of the genus Prevotella, a member of the genus Plesiomonas (such as Plesiomonas shigelloides), a member of the genus Pseudomonas (such as Pseudomonas aeruginosa), a member of the genus Providencia, a member of the genus Rickettsia (such as Rickettsia rickettsii or Rickettsia typhi), a member of the genus Stenotrophomonas (such as Stenotrophomonas maltophila), a member of the genus Staphylococcus (such as Staphylococcus aureus or Staphylococcus epidermidis), a member of the genus Streptococcus (such as Streptococcus viridans, Streptococcus pyogenes (group A), Streptococcus agalactiae (group B), Streptococcus bovis, or Streptococcus pneumoniae), a member of the genus Streptomyces (such as Streptomyces hygroscopicus), a member of the genus Salmonella (such as Salmonella enteriditis, Salmonella typhi, or Salmonella typhimurium), a member of the genus Serratia (such as Serratia marcescens), a member of the genus Shigella, a member of the genus Spirillum (such as Spirillum minus), a member of the genus Treponema (such as Treponema pallidum), a member of the genus Veillonella, a member of the genus Vibrio (such as Vibrio cholerae, Vibrio parahaemolyticus, or Vibrio vulnificus), a member of the genus Yersinia (such as Yersinia enterocolitica, Yersinia pestis, or Yersinia pseudotuberculosis), and a member of the genus Xanthomonas (such as Xanthomonas maltophilia).

Specifically, Bacillus subtilis forms architecturally complex communities on semi-solid surfaces and thick pellicles at the air/liquid interface of standing cultures (López et al., FEMS Microbiol. Rev. 33:152 (2009); Aguilar et al., Curr. Opin. Microbiol. 10:638 (2007); Vlamakis et al., Genes Dev. 22:945 (2008); Branda et al., Proc. Natl. Acad. Sci. USA 98:11621 (2001)). B. subtilis biofilms consist of long chains of cells held together by an extracellular matrix consisting of an exopolysaccharide and amyloid fibers composed of the protein TasA (Branda et al., Proc. Natl. Acad. Sci. USA 98:11621 (2001); Branda et al., Mol. Microbiol. 59:1229 (2006); Romero et al., Proc. Natl. Acad. Sci. USA (2010, in press)). The exopolysaccharide is produced by enzymes encoded by the epsA-O operon (“eps operon”) and the TasA protein is encoded by the promoter-distal gene of the yqxM-sipW-tasA operon (“yqxM operon”) (Chu et al., Mol. Microbiol. 59:1216 (2006)).

Biofilm-producing bacteria, e.g., a species described herein, can be found in a live subject, in vitro, or on a surface, as described herein.

### Applications/Formulations

Compositions containing polyamines, or combinations of polyamines and D-amino acids, can be used to reduce or prevent biofilm formation on non-biological semi-solid or solid surfaces. Such a surface can be any surface that may be prone to biofilm formation and adhesion of bacteria. Nonlimiting examples of surfaces include hard surfaces made from one or more of the following materials: metal, plastic, rubber, board, glass, wood, paper, concrete, rock, marble, gypsum and ceramic materials, such as porcelain, which optionally are coated, for example, with paint or enamel.

In certain embodiments, the surface is a surface that contacts with water or, in particular, with standing water. For example, the surface can be a surface of a plumbing system, industrial equipment, water condensate collectors, equipment used for sewer transport, water recirculation, paper pulping, and water processing and transport. Nonlimiting examples include surfaces of drains, tubs, kitchen appliances, countertops, shower curtains, grout, toilets, industrial food and beverage production facilities, and flooring. Other surfaces include marine structures, such as boats, piers, oil platforms, water intake ports, sieves, and viewing ports.

A polyamine, or a combination of a polyamine and D-amino acid, can be applied to a surface by any known means, such as by covering, coating, contacting, associating with, filling, or loading the surface with an effective amount of a polyamine, or a combination of a polyamine and D-amino acid. The a polyamine, or the combination of a polyamine and D-amino acid, can be applied to the surface with a suitable carrier, e.g., a fluid carrier, that is removed, e.g., by evaporation, to leave a polyamine coating, or a coating containing a combination of a polyamine and D-amino acid. In specific examples, a polyamine, or a combination of a polyamine and D-amino acid, is directly affixing to a surface by either spraying the surface, for example with a polymer/polyamine film, or a polymer/combination of polyamine and D-amino acid film, by dipping the surface into or spin-coating onto the surface, for example with a polymer/polyamine solution, or solution containing a polymer and combination of a polyamine and D-amino acid, or by other covalent or noncovalent means. In other instances, the surface is coated with an absorbant substance (such as a hydrogel) that absorbs the polyamine, or the combination of a polyamine and D-amino acid.

The polyamines, or combinations of polyamines and D-amino acids, are suitable for treating surfaces in a hospital or medical setting. Application of the polyamines, or combinations of polyamines and D-amino acids, and compositions described herein can inhibit biofilm formation or reduce biofilm formation when applied as a coating, lubricant, washing or cleaning solution, etc.

The polyamines, or combinations of polyamines and D-amino acids, described herein are also suitable for treating, especially preserving, textile fibre materials. Such materials are undyed and dyed or printed fibre materials, e.g. of silk, wool, polyamide or polyurethanes, and especially cellulosic fibre materials of all kinds. Such fibre materials are, for example, natural cellulose fibres, such as cotton, linen, jute and hemp, as well as cellulose and regenerated cellulose. Paper, for example paper used for hygiene purposes, may also be provided with antibiofilm properties using one or more polyamines, or combinations of polyamines and D-amino acids, described herein. It is also possible for nonwovens, e.g. nappies/diapers, sanitary towels, panty liners, and cloths for hygiene and household uses, to be provided with antibiofilm properties.

The polyamines, or combinations of polyamines and D-amino acids, described herein are suitable also for treating, especially imparting antibiofilm properties to or preserving industrial formulations such as coatings, lubricants etc.

The polyamines, or combinations of polyamines and D-amino acids, described herein can also be used in washing and cleaning formulations, e.g. in liquid or powder washing agents or softeners. The polyamines, or combinations of polyamines and D-amino acids, described herein can also be used in household and general-purpose cleaners for cleaning and disinfecting hard surfaces. An exemplary cleaning preparation has, for example, the following composition: 0.01 to 5% by weight of one or more polyamines, or combinations of polyamines and D-amino acids, 3.0% by weight octyl alcohol 4EO, 1.3% by weight fatty alcohol C₈-C₁₀ polyglucoside, 3.0% by weight isopropanol, and water ad 100%.

The polyamines, or combinations of polyamines and D-amino acids, described herein can also be used for the antibiofilm treatment of wood and for the antibiofilm treatment of leather, the preserving of leather and the provision of leather with antibiofilm properties. The polyamines, or combinations of polyamines and D-amino acids, described herein can also be used for the protection of cosmetic products and household products from microbial damage.

The polyamines, or combinations of polyamines and D-amino acids, described herein are useful in preventing bio-fouling, or eliminating or controlling microbe accumulation on the surfaces either by incorporating one or more polyamines, or combinations of polyamines and D-amino acids, described herein into the article or surface of the article in question or by applying the antibiofilm to these surfaces as part of a coating or film. Such surfaces include surfaces in contact with marine environments (including fresh water, brackish water and salt water environments), for example, the hulls of ships, surfaces of docks or the inside of pipes in circulating or pass-through water systems. Other surfaces are susceptible to similar biofouling, for example walls exposed to rain water, walls of showers, roofs, gutters, pool areas, saunas, floors and walls exposed to damp environs such as basements or garages and even the housing of tools and outdoor furniture. U.S. Pat. No. 7,618,697, which is hereby incorporated in its entirety by reference, discloses compounds useful in coatings or films in protecting surfaces from bio-fouling.

When applied as a part of a film or coating, one or more polyamines, or combinations of polyamines and D-amino acids, described herein can be part of a composition which also comprises a binder. The binder may be any polymer or oligomer compatible with the present antibiofilms. The binder may be in the form of a polymer or oligomer prior to preparation of the anti-fouling composition, or may form by polymerization during or after preparation, including after application to the substrate. In certain applications, such as certain coating applications, it will be desirable to crosslink the oligomer or polymer of the anti fouling composition after application. The term “binder” as used herein also includes materials such as glycols, oils, waxes and surfactants commercially used in the care of wood, plastic, glass and other surfaces. Examples include water proofing materials for wood, vinyl protectants, protective waxes and the like.

The composition can be a coating or a film. When the composition is a thermoplastic film which is applied to a surface, for example, by the use of an adhesive or by melt applications including calendaring and co-extrusion, the binder is the thermoplastic polymer matrix used to prepare the film. When the composition is a coating, it may be applied as a liquid solution or suspension, a paste, gel, oil or the coating composition may be a solid, for example a powder coating which is subsequently cured by heat, UV light or other method.

As the composition of the invention may be a coating or a film, the binder can be comprised of any polymer used in coating formulations or film preparation. For example, the binder is a thermoset, thermoplastic, elastomeric, inherently crosslinked or crosslinked polymer. Thermoset, thermoplastic, elastomeric, inherently crosslinked or crosslinked polymers include polyolefin, polyamide, polyurethane, polyacrylate, polyacrylamide, polycarbonate, polystyrene, polyvinyl acetates, polyvinyl alcohols, polyester, halogenated vinyl polymers such as PVC, natural and synthetic rubbers, alkyd resins, epoxy resins, unsaturated polyesters, unsaturated polyamides, polyimides, silicon containing and carbamate polymers, fluorinated polymers, crosslinkable acrylic resins derived from substituted acrylic esters, e.g., from epoxy acrylates, urethane acrylates or polyester acrylates. The polymers may also be blends and copolymers of the preceding chemistries.

Biocompatible coating polymers, such as, poly[-alkoxyalkanoate-co-3-hydroxyalkenoate] (PHAE) polyesters, Geiger et. al. Polymer Bulletin 52, 65-70 (2004), can also serve as binders in the present invention. Alkyd resins, polyesters, polyurethanes, epoxy resins, silicone containing polymers, polyacrylates, polyacrylamides, fluorinated polymers and polymers of vinyl acetate, vinyl alcohol and vinyl amine are non-limiting examples of common coating binders useful in the present invention. Other known coating binders are part of the present disclosure.

Coatings can be crosslinked with, for example, melamine resins, urea resins, isocyanates, isocyanurates, polyisocyanates, epoxy resins, anhydrides, poly acids and amines, with or without accelerators. The compositions described herein can be, for example, a coating applied to a surface which is exposed to conditions favorable for bioaccumulation. The presence of one or more polyamines, or combinations of polyamines and D-amino acids, described herein in said coating can prevent the adherence of organisms to the surface.

The polyamines, or combinations of polyamines and D-amino acids, described herein can be part of a complete coating or paint formulation, such as a marine gel-coat, shellac, varnish, lacquer or paint, or the anti fouling composition may comprise only a polymer of the instant invention and binder, or a polymer of the instant invention, binder and a carrier substance. Other additives known in the art in such coating formulations or applications are also suitable.

The coating may be solvent borne or aqueous. Aqueous coatings are typically considered more environmentally friendly. In some examples, the coating can be an aqueous dispersion of one or more polyamines, or combinations of polyamines and D-amino acids, and a binder or a water based coating or paint. For example, the coating can comprise an aqueous dispersion of one or more polyamines, or combinations of polyamines and D-amino acids, and an acrylic, methacrylic or acrylamide polymers or co-polymers or a poly[-alkoxyalkanoate-co-3-hydroxyalkenoate] polyester.

The coating can be applied to a surface which has already been coated, such as a protective coating, a clear coat or a protective wax applied over a previously coated article. Coating systems include marine coatings, wood coatings, other coatings for metals and coatings over plastics and ceramics. Exemplary of marine coatings are gel coats comprising an unsaturated polyester, a styrene and a catalyst. In some examples, the coating is a house paint, or other decorative or protective paint. It can be a paint or other coating that is applied to cement, concrete or other masonry article. The coating may be a water proofer as for a basement or foundation.

In some instances, the coating composition can be applied to a surface by any conventional means including spin coating, dip coating, spray coating, draw down, or by brush, roller or other applicator. A drying or curing period can be performed.

Coating or film thickness can vary depending on the application and can readily be determined by one skilled in the art after limited testing.

In some instances, a composition described herein can be in the form of a protective laminate film. Such a film can comprise thermoset, thermoplastic, elastomeric, or crosslinked polymers. Examples of such polymers include, but are not limited to, polyolefin, polyamide, polyurethane, polyacrylate, polyacrylamide, polycarbonate, polystyrene, polyvinyl acetates, polyvinyl alcohols, polyester, halogenated vinyl polymers such as PVC, natural and synthetic rubbers, alkyd resins, epoxy resins, unsaturated polyesters, unsaturated polyamides, polyimides, fluorinated polymers, silicon containing and carbamate polymers. The polymers can also be blends and copolymers of the preceding chemistries.

When a composition described herein is a preformed film, it can be applied to a surface by, for example, the use of an adhesive, or co-extruded onto the surface. It can also be mechanically affixed via fasteners which may require the use of a sealant or caulk wherein the esters of the instant invention may also be advantageously employed. A plastic film can also be applied with heat which includes calendaring, melt applications and shrink wrapping.

In other instances, a composition described herein can be part of a polish, such a furniture polish, or a dispersant or surfactant formulation such as a glycol or mineral oil dispersion or other formulation as used in for example wood protection. Examples of useful surfactants include, but are not limited to, polyoxyethylene-based surface-active substances, including polyoxyethylene sorbitan tetraoleate (PST), polyoxyethylene sorbitol hexaoleate (PSH), polyoxyethylene 6 tridecyl ether, polyoxyethylene 12 tridecyl ether, polyoxyethylene 18 tridecyl ether, TWEEN® surfactants, TRITON® surfactants, and the polyoxyethlene-polyoxypropylene copolymers such as the PLURONIC® and POLOXAMER® product series (from BASF). Other matrix-forming components include dextrans, linear PEG molecules (MW 500 to 5,000,000), star-shaped PEG molecules, comb-shaped and dendrimeric, hyperbranched PEG molecules, as well as the analogous linear, star, and dendrimer polyamine polymers, and various carbonated, perfluorinated (e.g., DUPONT ZONYL® fluorosurfactants) and siliconated (e.g, dimethylsiloxane-ethylene oxide block copolymers) surfactants.

Given the wide array of applications for the polyamines, or combinations of polyamines and D-amino acids, described herein, a composition containing a polyamine, or a composition containing a combination of a polyamine and a D-amino acid, can include other additives such as antioxidants, UV absorbers, hindered amines, phosphites or phosphonites, benzofuran-2-ones, thiosynergists, polyamide stabilizers, metal stearates, nucleating agents, fillers, reinforcing agents, lubricants, emulsifiers, dyes, pigments, dispersants, other optical brighteners, flame retardants, antistatic agents, blowing agents and the like, such as the materials listed below, or mixtures thereof.

The substrate to be treated can be an inorganic or organic substrate, for example, a metal or metal alloy; a thermoplastic, elastomeric, inherently crosslinked or crosslinked polymer as described above; a natural polymer such as wood or rubber; a ceramic material; glass; leather or other textile. The substrate may be, for example, non-metal inorganic surfaces such as silica, silicon dioxide, titanium oxides, aluminum oxides, iron oxides, carbon, silicon, various silicates and sol-gels, masonry, and composite materials such as fiberglass and plastic lumber (a blend of polymers and wood shavings, wood flour or other wood particles).

The substrate can be a multi-layered article comprised of the same or different components in each layer. The surface coated or laminated may be the exposed surface of an already applied coating or laminate.

The inorganic or organic substrate to be coated or laminated can be in any solid form.

For example, polymer substrates may be plastics in the form of films, injection-molded articles, extruded workpieces, fibres, felts or woven fabrics. For example, molded or extruded polymeric articles used in construction or the manufacture of durable goods such as siding, fascia and mailboxes can all benefit from incorporation of the present polyamines, or combinations of polyamines and D-amino acids. In certain situations, one or more polyamines, or combinations of polyamines and D-amino acids, can be incorporated into the polymeric article during the forming, e.g., molding process.

Plastics which would benefit from the present method include, but are not limited to, plastics used in construction or the manufacture of durable goods or machine parts, including outdoor furniture, boats, siding, roofing, glazing, protective films, decals, sealants, composites like plastic lumber and fiber reinforced composites, functional films including films used in displays as well as articles constructed from synthetic fibers such as awnings, fabrics such as used in canvas or sails and rubber articles such as outdoor matting, floor coverings, plastics coatings, plastics containers and packaging materials; kitchen and bathroom utensils (e.g. brushes, shower curtains, sponges, bathmats), latex, filter materials (air and water filters), plastics articles used in the field of medicine, e.g. dressing materials, syringes, catheters, etc., so-called “medical devices”, gloves and mattresses. Exemplary of such plastics are polypropylene, polyethylene, PVC, POM, polysulfones, polyethersulfones, polystyrenics, polyamides, polyurethanes, polyesters, polycarbonate, polyacrylics and methacrylics, polybutadienes, thermoplastic polyolefins, ionomers, unsaturated polyesters and blends of polymer resins including ABS, SAN and PC/ABS.

In certain situations, such as incorporation of one or more polyamines, or combinations of polyamines and D-amino acids, described herein into recirculating cooling water, a few parts per million of the polyamines, or combinations of polyamines and D-amino acids, are effective to prevent biofilm accumulation on the walls of pipes and other mechanical apparatus. However, some loss due to leaching, some loss due to reactions involving the amino acids and some loss to degradation reactions, etc., means that in practice one can prepare formulations having concentrations that will be effective over the period of time envisioned for the application and taking into account the environmental stresses the polyamines, or combinations of polyamines and D-amino acids, will be exposed to.

For example, in industrial water applications, about 0.001% to about 10% by weight or for example 0.001% to 10% by weight, of one or more polyamines, or combinations of polyamines and D-amino acids, relative to the water being treated can be used, often, an upper limit of less than about 10% can be used, for example about 5%, about 3%, about 2% or even about 1% or less can be effective in many circumstances, for example, load levels of about 0.01% to about 5%, or about 0.01% to about 2% of one or more polyamines, or combinations of polyamines and D-amino acids, can be used. In other embodiments, an upper limit of less than 10%, 5%, 3%, 2%, 1%, can be used, such as 0.01% to 5%, or about 0.01% to 2% by weight of one or more polyamines, or combinations of polyamines and D-amino acids, can be used. Given the high activity of the instant polyamines, or combinations of polyamines and D-amino acids, very small amounts are effective in many circumstances and concentrations of about 0.000001% to about 0.5%, for example, about 0.000001% to about 0.1% or, about 0.000001% to about 0.01% can be used in industrial water applications. In other embodiments, concentrations of 0.000001% to 0.5%, for example, 0.000001% to 0.1% or 0.000001% to 0.01% can be used in industrial water applications

The polyamines, or combinations of polyamines and D-amino acids, especially in low concentrations, can be safely used even in applications where ingestion is possible, such as reusable water bottles or drinking fountains where a biofilm may develop. The surfaces of such water transport devices can be rinsed with a formulation containing one or more polyamines, or combinations of polyamines and D-amino acids, or low levels of one or more polyamines, or combinations of polyamines and D-amino acids, can be introduced into the water that passes through the containers of conduits. For example, about 0.0001% or less or up to about 1%, typically less than about 0.1% by weight of one or more polyamines, or combinations of polyamines and D-amino acids, may be introduced into such water. In other examples, 0.0001% or less or up to 1%, typically less than 0.1% by weight of one or more polyamines, or combinations of polyamines and D-amino acids, may be introduced into such water. Given the high activity of the instant polyamines, or combinations of polyamines and D-amino acids, very small amounts are effective in many circumstances and concentrations of about 0.000001% to about 0.1%, for example, about 0.000001% to about 0.01%, or about 0.000001% to about 0.001% can be used in such applications. In other examples, concentrations of 0.000001% to 0.1%, 0.000001% to 0.01%, or 0.000001% to 0.001% can be used.

In some instances, liquid formulations are prepared at about 0.0005 μM polyamine to about 50 μM polyamine, e.g., about 0.001 μM polyamine to about 25 μM polyamine, about 0.002 μM polyamine to about 10 μM polyamine, about 0.003 μM polyamine to about 5 μM polyamine, about 0.004 μM polyamine to about 1 μM polyamine, about 0.005 μM polyamine to about 0.5 μM polyamine, about 0.01 μM polyamine to about 0.1 μM polyamine, or about 0.02 μM polyamine to about 0.1 μM polyamine. In other embodiments, the liquid formulation is prepared at 0.0005 μM polyamine to 50 μM polyamine, 0.001 μM polyamine to 25 μM polyamine, 0.002 μM polyamine to 10 μM polyamine, 0.003 μM polyamine to 5 μM polyamine, 0.004 μM polyamine to 1 μM polyamine, 0.005 μM polyamine to 0.5 μM polyamine, 0.01 μM polyamine to 0.1 μM polyamine, or 0.02 μM polyamine to 0.1 μM polyamine.

In some instances, liquid formulations are prepared at about 0.0005 μM D-amino acid to about 50 μM D-amino acid, e.g., about 0.001 μM D-amino acid to about 25 μM D-amino acid, about 0.002 μM D-amino acid to about 10 μM D-amino acid, about 0.003 μM D-amino acid to about 5 μM D-amino acid, about 0.004 μM D-amino acid to about 1 μM D-amino acid, about 0.005 μM D-amino acid to about 0.5 μM D-amino acid, about 0.01 μM D-amino acid to about 0.1 μM D-amino acid, or about 0.02 μM D-amino acid to about 0.1 μM D-amino acid. In other embodiments, the liquid formulation is prepared at 0.0005 μM D-amino acid to 50 μM D-amino acid, 0.001 μM D-amino acid to 25 μM D-amino acid, 0.002 μM D-amino acid to 10 μM D-amino acid, 0.003 μM D-amino acid to 5 μM D-amino acid, 0.004 μM D-amino acid to 1 μM D-amino acid, 0.005 μM D-amino acid to 0.5 μM D-amino acid, 0.01 μM D-amino acid to 0.1 μM D-amino acid, or 0.02 μM D-amino acid to 0.1 μM D-amino acid. Preferably, the a D-amino acid composition is at nanomolar concentrations, e.g., at about 5 nM, at about 10 nM, at about 15 nM, at about 20 nM, at about 25 nM, at about 30 nM, at about 50 nM, or more. In other embodiments, the D-amino acid composition is bout 5 nM, at 10 nM, at 15 nM, at 20 nM, at 25 nM, at 30 nM, or at 50 nM.

When used in a coating or film, small amounts of one or more polyamines, or combinations of polyamines and D-amino acids, can be present for short term use, for example, one use, seasonal or disposable items, etc. In general, about 0.001% or less up to about 5%, for example up to about 3% or about 2% may be used in such coatings or films. In other embodiments, 0.001% to 5%, or up to 3% or 2% by weight of one or more polyamines, or combinations of polyamines and D-amino acids, may be used. Given the high activity of the instant polyamines, or combinations of polyamines and D-amino acids, very small amounts are effective in many circumstances and concentrations of about 0.0001% to about 1%, for example, about 0.0001% to about 0.5%, or about 0.0001% to about 0.01% can be used in coating applications. In other embodiments, concentrations of 0.0001% to 1%, 0.0001% to 0.5%, or 0.0001% to 0.01% by weight of one or more polyamines, or combinations of polyamines and D-amino acids, can be used in coating applications.

For more robust uses, for example, coatings for marine, pool, shower or construction materials, higher levels of one or more polyamines, or combinations of polyamines and D-amino acids, can be used. For example, from about 0.01% to about 30% based on the coating or film formulation can be employed; in many uses, about 0.01% to about 15%, or to about 10% will be effective, and often about 0.01% to about 5%, or about 0.01% to about 1%, or even about 0.1% or less polyamine, or combination of a polyamine and D-amino acid, can be used. In other embodiments, 0.01% to 15%, or 0.01% to 10% will be effective, and often 0.01% to 5%, or 0.01% to 1%, or even 0.1% of one or more polyamines, or combinations of polyamines and D-amino acids, can be used.

For incorporation into a molded plastic article, about 0.00001% to about 10% of one or more polyamines, or combinations of polyamines and D-amino acids, can be used, for example about 0.0001% to about 3%, for example about 0.001% up to about 1% one or more polyamines, or combinations of polyamines and D-amino acids, can be used. In some embodiments, 0.00001% to 10% of one or more polyamines, or combinations of polyamines and D-amino acids, can be used, or 0.0001% to 3%, or 0.001% up to 1% of one or more polyamines, or combinations of polyamines and D-amino acids, can be used. In situations in which the polyamines, or combinations of polyamines and D-amino acids, are impregnated into the surface of an already prepared molded article or fiber, the actual amount of a polyamine, or a combination of a polyamine and D-amino acid, present at the surface can depend on the substrate material, the formulation of the impregnating composition, and the time and temperature used during the impregnation step. Given the high activity of the instant polyamines, or combinations of polyamines and D-amino acids, very small amounts are effective in many circumstances, and concentrations of about 0.0001% to about 1%, for example, about 0.0001% to about 0.1%, or about 0.0001% to about 0.01% can be used in plastics. In other embodiments, 0.0001% to 1%, or 0.0001% to 0.1%, or 0.0001% to 0.01% by weight of one or more polyamines, or combinations of polyamines and D-amino acids, can be used in plastics

Inhibition or reduction in a biofilm by treatment with a polyamine, or a combination of a polyamine and D-amino acid, can be measured using techniques well established in the art. These techniques enable one to assess bacterial attachment by measuring the staining of the adherent biomass, to view microbes in vivo using microscopy methods; or to monitor cell death in the biofilm in response to toxic agents. Following treatment, the biofilm can be reduced with respect to the surface area covered by the biofilm, thickness, and consistency (for example, the integrity of the biofilm). Non-limiting examples of biofilm assays include microtiter plate biofilm assays, fluorescence-based biofilm assays, static biofilm assays according to Walker et al., Infect. Immun. 73:3693-3701 (2005), air-liquid interface assays, colony biofilm assays, and Kadouri Drip-Fed Biofilm assays (Merritt et al., (2005) Current Protocols in Microbiology 1.B.1.1-1.B.1.17). Such assays can be used to measure the activity of a polyamine, or a combination of a polyamine and D-amino acid, on the disruption or the inhibition of formation of a biofilm (Lew et al., (2000) Curr. Med. Chem. 7(6):663-72; Werner et al., (2006) Brief Funct. Genomic Proteomic 5(1):32-6).

### Combination Compositions and Methods

Biofilms are understood, very generally, to be aggregations of living and dead micro-organisms, especially bacteria, that adhere to living and non-living surfaces, together with their metabolites in the form of extracellular polymeric substances (EPS matrix), e.g. polysaccharides. The activity of antibiofilm substances that normally exhibit a pronounced growth-inhibiting or lethal action with respect to planktonic cells may be greatly reduced with respect to microorganisms that are organized in biofilms, for example because of inadequate penetration of the active substance into the biological matrix.

In some instances, a polyamine, or combination of a polyamine and D-amino acid, can be used in combination with a second agent, e.g., a biocide, an antibiotic, or an antimicrobial agent, to treat a biofilm or to prevent the formation of a biofilm. An antibiotic can be combined with the polyamine, or the combination of a polyamine and D-amino acid, either sequentially or simultaneously. For example, any of the compositions described herein can be formulated to include one or more polyamines, or combinations of polyamines and D-amino acids, and one or more second agents.

The antibiotic can be any compound known to one of ordinary skill in the art that can inhibit the growth of, or kill, bacteria. Useful, non-limiting examples of antibiotics include lincosamides (clindomycin); chloramphenicols; tetracyclines (such as Tetracycline, Chlortetracycline, Demeclocycline, Methacycline, Doxycycline, Minocycline); aminoglycosides (such as Gentamicin, Tobramycin, Netilmicin, Amikacin, Kanamycin, Streptomycin, Neomycin); beta-lactams (such as penicillins, cephalosporins, Imipenem, Aztreonam); glycopeptide antibiotics (such as vancomycin); polypeptide antibiotics (such as bacitracin); macrolides (erythromycins), amphotericins; sulfonamides (such as Sulfanilamide, Sulfamethoxazole, Sulfacetamide, Sulfadiazine, Sulfisoxazole, Sulfacytine, Sulfadoxine, Mafenide, p-Aminobenzoic Acid, Trimethoprim-Sulfamethoxazole); Methenamin; Nitrofurantoin; Phenazopyridine; trimethoprim; rifampicins; metronidazoles; cefazolins; Lincomycin; Spectinomycin; mupirocins; quinolones (such as Nalidixic Acid, Cinoxacin, Norfloxacin, Ciprofloxacin, Pefloxacin, Ofloxacin, Enoxacin, Fleroxacin, Levofloxacin); novobiocins; polymixins; gramicidins; and antipseudomonals (such as Carbenicillin, Carbenicillin Indanyl, Ticarcillin, Azlocillin, Mezlocillin, Piperacillin) or any salts or variants thereof. Such antibiotics are commercially available, e.g., from Daiichi Sankyo, Inc. (Parsipanny, N.J.), Merck (Whitehouse Station, N.J.), Pfizer (New York, N.Y.), Glaxo Smith Kline (Research Triangle Park, N.C.), Johnson & Johnson (New Brunswick, N.J.), AstraZeneca (Wilmington, Del.), Novartis (East Hanover, N.J.), and Sanofi-Aventis (Bridgewater, N.J.). The antibiotic used will depend on the type of bacterial infection.

Additional known biocides include biguanide, chlorhexidine, triclosan, chlorine dioxide, xylitol, and the like.

Useful examples of antimicrobial agents include, but are not limited to, Pyrithiones, especially the zinc complex (ZPT); Octopirox®; Dimethyldimethylol Hydantoin (Glydant®); Methylchloroisothiazolinone/methylisothiazolinone (Kathon CG®); Sodium Sulfite; Sodium Bisulfite; Imidazolidinyl Urea (Germall 115®, Diazolidinyl Urea (Germain II®); Benzyl Alcohol; 2-Bromo-2-nitropropane-1,3-diol (Bronopol®); Formalin (formaldehyde); Iodopropenyl Butylcarbamate (Polyphase P100®); Chloroacetamide; Methanamine; Methyldibromonitrile Glutaronitrile (1,2-Dibromo-2,4-dicyanobutane or Tektamer®); Glutaraldehyde; 5-bromo-5-nitro-1,3-dioxane (Bronidox®); Phenethyl Alcohol; o-Phenylphenol/sodium o-phenylphenol; Sodium Hydroxymethylglycinate (Suttocide A®); Polymethoxy Bicyclic Oxazolidine (Nuosept C®); Dimethoxane; Thimersal; Dichlorobenzyl Alcohol; Captan; Chlorphenenesin; Dichlorophene; Chlorbutanol; Glyceryl Laurate; Halogenated Diphenyl Ethers; 2,4,4′-trichloro-2′-hydroxy-diphenyl ether (Triclosan®. or TCS); 2,2′-dihydroxy-5,5′-dibromo-diphenyl ether; Phenolic Compounds; Phenol; 2-Methyl Phenol; 3-Methyl Phenol; 4-Methyl Phenol; 4-Ethyl Phenol; 2,4-Dimethyl Phenol; 2,5-Dimethyl Phenol; 3,4-Dimethyl Phenol; 2,6-Dimethyl Phenol; 4-n-Propyl Phenol; 4-n-Butyl Phenol; 4-n-Amyl Phenol; 4-tert-Amyl Phenol; 4-n-Hexyl Phenol; 4-n-Heptyl Phenol; Mono- and Poly-Alkyl and Aromatic Halophenols; p-Chlorophenol; Methyl p-Chlorophenol; Ethyl p-Chlorophenol; n-Propyl p-Chlorophenol; n-Butyl p-Chlorophenol; n-Amyl p-Chlorophenol; sec-Amyl p-Chlorophenol; Cyclohexyl p-Chlorophenol; n-Heptyl p-Chlorophenol; n-Octyl p-Chlorophenol; o-Chlorophenol; Methyl o-Chlorophenol; Ethyl o-Chlorophenol; n-Propyl o-Chlorophenol; n-Butyl o-Chlorophenol; n-Amyl o-Chlorophenol; tert-Amyl o-Chlorophenol; n-Hexyl o-Chlorophenol; n-Heptyl o-Chlorophenol; o-Benzyl p-Chlorophenol; o-Benxyl-m-methyl p-Chlorophenol; o-Benzyl-m; m-dimethyl p-Chlorophenol; o-Phenylethyl p-Chlorophenol; o-Phenylethyl-m-methyl p-Chlorophenol; 3-Methyl p-Chlorophenol; 3,5-Dimethyl p-Chlorophenol; 6-Ethyl-3-methyl p-Chlorophenol; 6-n-Propyl-3-methyl p-Chlorophenol; 6-iso-Propyl-3-methyl p-Chlorophenol; 2-Ethyl-3,5-dimethyl p-Chlorophenol; 6-sec-Butyl-3-methyl p-Chlorophenol; 2-iso-Propyl-3,5-dimethyl p-Chlorophenol; 6-Diethylmethyl-3-methyl p-Chlorophenol; 6-iso-Propyl-2-ethyl-3-methyl p-Chlorophenol; 2-sec-Amyl-3,5-dimethyl p-Chlorophenol; 2-Diethylmethyl-3,5-dimethyl p-Chlorophenol; 6-sec-Octyl-3-methyl p-Chlorophenol; p-Chloro-m-cresol: p-Bromophenol; Methyl p-Bromophenol; Ethyl p-Bromophenol; n-Propyl p-Bromophenol; n-Butyl p-Bromophenol; n-Amyl p-Bromophenol; sec-Amyl p-Bromophenol; n-Hexyl p-Bromophenol; Cyclohexyl p-Bromophenol; o-Bromophenol; tert-Amyl o-Bromophenol; n-Hexyl o-Bromophenol; n-Propyl-m,m-Dimethyl o-Bromophenol; 2-Phenyl Phenol; 4-Chloro-2-methyl phenol; 4-Chloro-3-methyl phenol; 4-Chloro-3,5-dimethyl phenol; 2,4-Dichloro-3,5-dimethylphenol; 3,4,5,6-Terabromo-2-methylphenol; 5-Methyl-2-pentylphenol; 4-Isopropyl-3-methylphenol; Para-chloro-meta-xylenol (PCMX); Chlorothymol; Phenoxyethanol; Phenoxyisopropanol; 5-Chloro-2-hydroxydiphenylmethane; Resorcinol and its Derivatives; Resorcinol; Methyl Resorcinol;
Ethyl Resorcinol; n-Propyl Resorcinol; n-Butyl Resorcinol; n-Amyl Resorcinol; n-Hexyl Resorcinol; n-Heptyl Resorcinol; n-Octyl Resorcinol; n-Nonyl Resorcinol; Phenyl Resorcinol; Benzyl Resorcinol; Phenylethyl Resorcinol; Phenylpropyl Resorcinol; p-Chlorobenzyl Resorcinol; 5-Chloro 2,4-Dihydroxydiphenyl Methane; 4′-Chloro 2,4-Dihydroxydiphenyl Methane; 5-Bromo 2,4-Dihydroxydiphenyl Methane; 4′-Bromo 2,4-Dihydroxydiphenyl Methane; Bisphenolic Compounds; 2,2′-Methylene bis(4-chlorophenol); 2,2′-Methylene bis-(3,4,6-trichlorophenol); 2,2′-Methylene bis(4-chloro-6-bromophenol); bis(2-hydroxy-3,5-dichlorophenyl)sulfide; bis(2-hydroxy-5-chlorobenzyl)sulfide; Benzoic Esters (Parabens); Methylparaben; Propylparaben; Butylparaben; Ethylparaben; Isopropylparaben; Isobutylparaben; Benzylparaben; Sodium Methylparaben; Sodium Propylparaben; Halogenated Carbanilides; 3,4,4′-Trichlorocarbanilides (Triclocarban® or TCC); 3-Trifluoromethyl-4,4′-dichlorocarbanilide; 3,3′,4-Trichlorocarbanilide; Chlorohexidine and its digluconate; diacetate and dihydrochloride; Undecenoic acid; thiabendazole, Hexetidine; poly(hexamethylenebiguanide) hydrochloride (Cosmocil®); silver compounds such as organic silver salts it anorganic silver salts, silver chloride including formulations thereof such as JM Acticare® and micronized silver particles.

The invention is further described in the following examples, which do not limit the scope of the invention described in the claims. Room temperature denotes a temperature from the range of 20-25° C.

### EXAMPLES

The Following Materials and Methods were Used in Examples 1-7

Strains and Media.

Bacillus subtilis strains PY79, 3610 and their derivatives were grown in Luria-Bertani (LB) medium at 37° C. or MSgg medium (1) at 23° C. Solid media contained 1.5% Bacto agar. When appropriate, antibiotics were added at the following concentrations for growth of B. subtilis: 10 μg per ml of tetracycline, and 5 μg per ml of erythromycin, 500 μg per ml of spectinomycin.

Strains used:
PY79: a derivative of B. subtilis 168, was used as a host for transformation;
3610: a wild strain of B. subtilis (NCBI 3610), which is capable of forming robust biofilms (1);
Staphylococcus aureus SCO1 was obtained from the Kolter lab collection (2);
E. coli strain MC4100 was obtained from the Kolter lab collection;
Pseudomonas aeruginosa PAH was obtained from the Kolter lab collection;
Strain DS76: 3610 Δeps::tet (lab stock);
Strain FC55: 3610 ΔtasA::spec (lab stock);
Strain FC5: 3610 containing P_{epsA}-lacZ at the amyE locus and a cat gene;
Strain IKG55: 3610 containing Δrac X::spec and ΔylmE::tet (3);
Strain DR30: 3610 containing tasA-mCherry at the amyE locus and a cat gene;
Strain IKG624: 3610 containing ΔyaaO::tet (this work);
Strain IKG623: 3610 containing ΔgabT::spec (this work);
Strain IKG625: 3610 containing ΔracX::mls and ΔylmE::tet, ΔgbaT::kan (this work);
Strain IKG626: 3610 containing ΔracX::spec and ΔylmE::mis, ΔyaaO::tet (this work).

Strain Construction:

Strains were constructed using standard methods (4). Long-flanking PCR mutagenesis was used to create ΔgbaT::spec and ΔyaaO (5). Primers are described in the table below. DNA was introduced into lab strains by DNA-mediated transformation of competent cells (6). SPP1 phage-mediated transduction was used to move antibiotic resistance marker-linked mutations from lab strains to the wild strain 3610 (7).

### Compounds and Reagents:

Norspermidine (1), norspermine (2), spermidine (3), spermine (4), putrescine (5), 1,3-diaminopropane (6), 1,1,5,9,9-N,N,N,N,N-pentamethylnorspermidine [N¹-(3-(dimethylamino)propyl)-N¹,N³,N³-trimethylpropane-1,3-diamine, 7], 1,1,9,9-N,N,N,N-tetramethylnorspermidine [N¹-(3-(dimethylamino)propyl)-N³, N³-dimethylpropane-1,3-diamine, (8), 5-N-methylnorspermidine [(3-aminopropyl)-N¹-methylpropane-1,3-diamine, (9), 3,3′-oxybis(propan-1-amine) (10), 3,7,11,18,22,26-Hexaazatricyclo[26.2.2.213,16]tetratriaconta-13,15,28,30,31,33-hexaene (12), N¹-dodecyl-N³-(3-(dodecylamino)propyl)propane-1,3-diamine (13), di-tert-butyl 5,5′-((3,4-dimethyl-1H-pyrrole-2,5-diyl)bis(methylene))bis(3,4-dimethyl-1H-pyrrole-2-carboxylate) (14), 2,2′-(1H-pyrrole-2,5-diyl)di(acetohydrazide) (15), 3,3′-(2,2-dimethylhydrazine-1,1-diyl)dipropanamide (16), N,N′-(azanediylbis(propane-3,1-diyl))bis(2,3,4,5,6-pentahydroxyhexanamide) (18), pentane-1,5-diamine (19), 3,3′-azanediylbis(propan-1-ol) (20), and N¹,N¹-bis(3-aminopropyl)propane-1,3-diamine (21) were obtained from Sigma-Aldrich (Atlanta, Ga.). N,N′-(azanediylbis(propane-3,1-diyl))bis(2,3,4,5,6-pentahydroxyhexanamide) (17) was purchased from Toronto research chemicals (Toronto, Canada). Diethyl 4-oxoheptanedioate (11) was synthesized according to the literature from furylacrylic acid (8). The product was purified by distillation as described previously and identified by ¹H NMR and ¹³C NMR. Texas-Red-Concanavalin A was obtained from Invitrogen-Molecular Probes (Eugene, Oreg.).

### Example 1

### Identification of Norspermidine in Conditioned Medium from B. Subtilis and the Effect of Norspermidine on Pellicle Formation

B. subtilis strain NCBI3610 was grown at 22° C. in 12-well plates in liquid biofilm-inducing medium for 3 or 8 days. Conditioned medium (500 ml) from an 8-day-old culture was concentrated on the C-18 column and eluted step-wise with methanol. Shown in FIG. 1A is the result of growing cells in fresh medium to which had been added 20 μl of the 25%, 35% or 40% methanol eluates. The 25% and 40% eluates contained compounds active in inhibiting biofilm formation whereas the 35% eluate was inert (FIG. 1A).

As reported previously, the factor in the 40% eluate was a mixture of D-amino acids (Kolodkin-Gal et al., 2010). To identify the second biofilm-inhibiting factor, high-performance liquid chromatography (HPLC) was carried out on the 25% methanol eluate using a phenyl-hexyl column. Inhibitory activity was recovered with an elution time of 40 min. Proton NMR analysis of the active fraction revealed fatty acids, morpholine and norspermidine. Cells of NCBI3610 were grown in fresh medium containing PBS buffer (control), norspermidine (100 μM), morpholine (100 μM) HPLC-purified fatty acid (˜100 μM), or spermidine (100 μM). Brighter images of the norspermidine-treated cell revealed cells near the bottom of the well. Further purification using a C-18 HPLC column identified the inhibitory agent as norspermidine, a finding confirmed with authentic norspermidine, which inhibited biofilm formation at 25 uM (FIG. 1B, FIG. 2A). Pure morpholine and fatty acids detected by NMR were inactive (FIG. 1B).

Norspermidine's tendency to form strong complexes with fatty acids, which explains its elution at 25% methanol from the C-18 Sep-Pak column, complicated its quantification. To circumvent this problem we treated conditioned medium with 9-fluorenylmethyloxycarbonyl chloride (Fmoc-Cl), which protects the amino groups of norspermidine as carbamates and thereby prevents their interaction with other molecules (Molnar-Perl, 2003). Pellicles were collected from 3- and 8-day-old cultures (100 ml) of the wild type (NCBI3610) and from an 8-day-old culture (100 ml) of a gab T mutant (IKG623). After mild sonication of the pellicles, cells were separated from extracellular material. Norspermidine in the extracellular material was derivatized with Fmoc-Cl and the resulting Fmoc-norspermidine was detected using an Agilent LC/MS system. (FIG. 1C). Fmoc-norspermidine was detectable in the old pellicle from wild type cells but not in the young or mutant pellicles. See also FIG. 2B. Norspermidine was present at a concentration of 50-80 μM in 8-day-old disassembling pellicles but at a concentration of less than 1 μM in a 3-day old pellicle (FIG. 1C).

Pellicle formation of strain NCBI 3610 was tested in the presence of the indicated concentrations of norspermidine (FIG. 1D) or spermidine (FIG. 1E). FIG. 2A also shows the results of testing of pellicle formation of strain NCBI 3610 in the presence of various concentrations of norspermidine. The effect of norspermidine was specific in that a closely related polyamine, spermidine, which differs from norspermidine by the presence of an extra methylene group, was inactive in inhibiting biofilm formation at concentrations up to 2 mM (FIGS. 1D and 1E).

### Example 2

### Assessment of Biofilms when Production of D-Amino Acid and Norspermidine Production is Blocked

To evaluate the contribution of norspermidine to biofilm disassembly genetically, a mutant blocking the production of norspermidine was created. Norspermidine is synthesized from aspartate-β-semialdehyde in a pathway involving the enzyme L-diaminobutyric acid transaminase (Lee et al., 2009). A mutant lacking the B. subtilis gene (gabT) encoding this enzyme was constructed and it was found that the enzyme was blocked in norspermidine production (FIG. 1C) and was partially impaired in biofilm disassembly (FIG. 3A). The gabT mutant formed pellicles that remained relatively thick at a time (day 7) when the wild type had undergone substantial disassembly. Nonetheless, the mutant pellicle had lost the wrinkly phenotype characteristic of young biofilms by day 7.

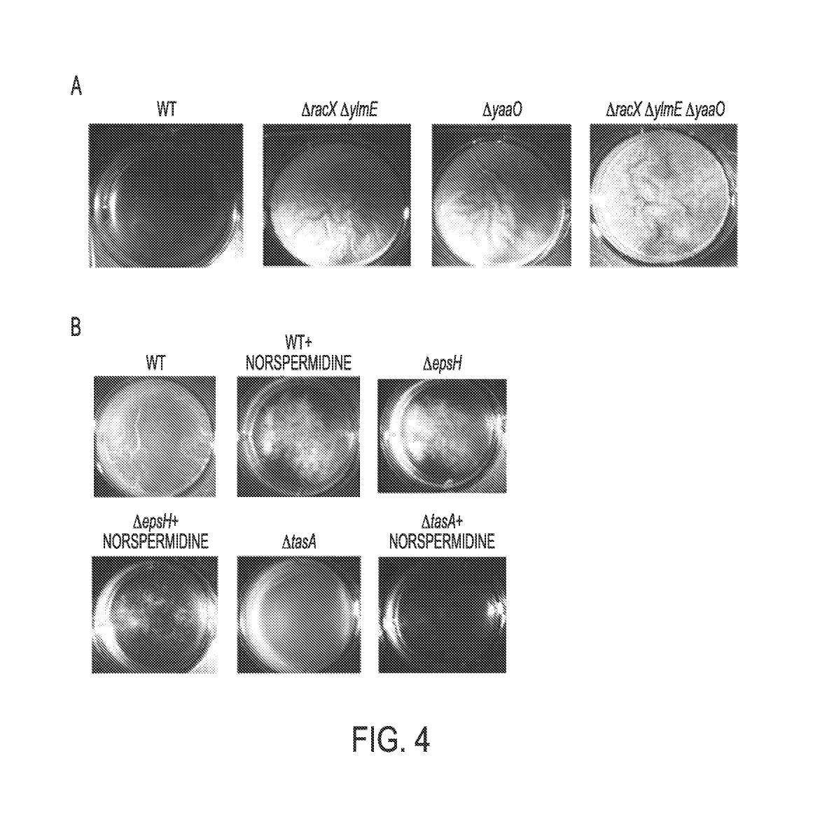

Further evaluation was conducted to assess whether the contribution of norspermidine to biofilm disassembly might be partially redundant with that of D-amino acids, which are produced by racemases encoded by racX and ylmE. Like a gabT mutant, a racX ylmE double mutant was partially impaired in biofilm disassembly. Strikingly, however, a gabT racX ylmE triple mutant formed robust pellicles that retained their wrinkly phenotype at a time (7 days) when the wild type had substantially disassembled (FIG. 3A). As a further test of the involvement of norspermidine in biofilm disassembly, a mutant lacking carboxynorspermidine decarboxylase which catalyzes the last step in the biosynthetic pathway was constructed. As in the case of the gabT mutant, a mutant lacking the B. subtilis homolog (yaaO) of the decarboxylase gene was partially impaired in biofilm disassembly and a yaaO racX ylmE triple mutant formed pellicles that remained intact at a time when the wild type had disassembled (FIG. 4A).

### Example 3

### D-Amino Acids and Norspermidine Act Through Different Mechanisms in Preventing Biofilm Formation and Triggering Biofilm Disassembly

To support the discovery that that norspermidine and D-amino acids act by different mechanisms to trigger biofilm disassembly, combinations of norspermidine with D-Tyr or with a mixture of D-Met, D-Trp, D-Leu and D-Tyr effectively prevented biofilm formation at concentrations that were ineffective in blocking biofilm formation when applied separately (FIG. 3B).

To assess whether the combination was more effective than norspermidine or D-amino acids alone, on the surface of 3 day-old pellicles were placed droplets (50 μl) containing buffer (PBS), a mixture of D-tyrosine, D-methionine, D-leucine and D-tryptophan each at final concentration of 12.5 μM, norspermidine at a final concentration of 50 μM, or, as in FIG. 3C, a combination of D-amino acids each at a concentration of only 2.5 μM and norspermidine at a concentration of 10 μM. After incubation for the indicated times, pellicle material and the medium were separated and each brought to a volume of 3 ml. After mild sonication, the OD₆₀₀ was determined for each sample. The % of disassembly represents the OD₆₀₀ of the medium as a percent of the sum of the OD₆₀₀ of the medium and the OD₆₀₀ of the pellicle. A mixture of D-amino acids and norspermidine was more effective in causing the breakdown of an existing biofilm than were either D-amino acids or norspermidine alone (FIG. 3C). Thus, D-amino acids and norspermidine act synergistically in preventing biofilm formation and triggering the disassembly of mature biofilms.

### Example 4

### Investigation of the Mechanism by which Polyamines Inhibit the Formation of and Trigger Disassembly of Biofilms

It was observed that the residual pellicle (wispy fragments of floating material with some structure) produced in the presence of norspermidine resembled pellicles seen for a mutant blocked in exopolysaccharide production but not those seen (thin, featureless pellicles) for a mutant blocked in amyloid-fiber production. More importantly, norspermidine had little effect on the residual pellicle produced by an exopolysaccharide mutant but abolished pellicle formation by the amyloid fiber mutant. In other words, the effect of norspermidine was synergistic with that of a mutation blocking fiber formation but not with a mutant blocked in exopolysaccharide production (FIG. 4B).

These observations suggested that norspermidine was interfering with the exopolysaccharide component of the matrix.

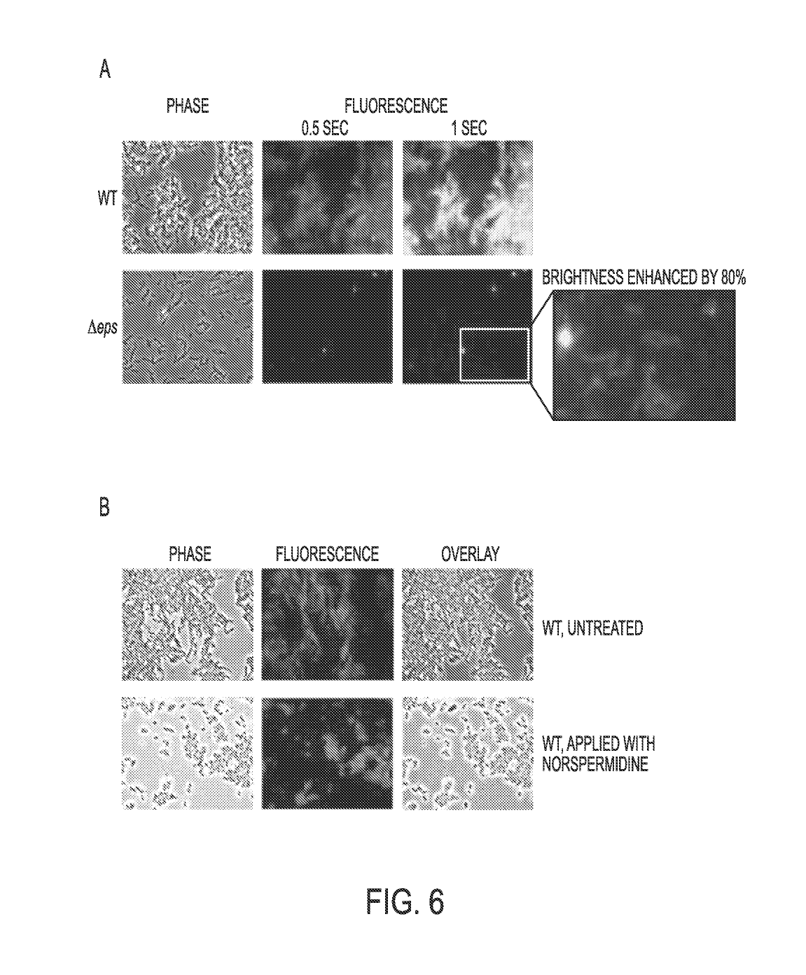

To investigate further, exopolysaccharide was visualized by fluorescence microscopy using a conjugate of the carbohydrate-binding protein concanavalin A with Texas Red (FIG. 5) (McSwain et al., 2005). As evidence of specificity, the conjugate decorated wild-type cells but not cells from a mutant (Δeps) blocked in exopolysaccharide production (FIG. 6A). Indeed, at an exposure at which concanavalin A staining with the wild type strain gave an extremely bright fluorescent signal, little or no signal could be detected for the Δeps mutant, except at long exposures and enhanced brightness (FIG. 6A). Next, we investigated the effects of norspermidine and spermidine. FIG. 5 shows that norspermidine treatment disrupted the relatively uniform, cell-associated pattern of staining seen with untreated cells, resulting in isolated patches of fluorescence. No such effect was seen with cells treated with spermidine. Simply mixing norspermidine with concanavalin A did not quench the intensity of fluorescence of the fluorophore (data not shown). Therefore, the difference in the staining was evidently due to differential levels of cell-associated exopolysaccharide. As a control, and in contrast to the results seen with concanavalin A, norspermidine had little or no effect on the protein component of the matrix as judged using a functional fusion of TasA to the fluorescent protein mCherry (FIG. 6B) (Kolodkin-Gal et al., 2010). Thus, while not being bound to any theory, it is believed that norspermidine disrupts the matrix and by targeting exopolysaccharide.

### Example 5

### Evaluation of Whether Norspermidine and Spermidine Interact with Exopolysaccharide

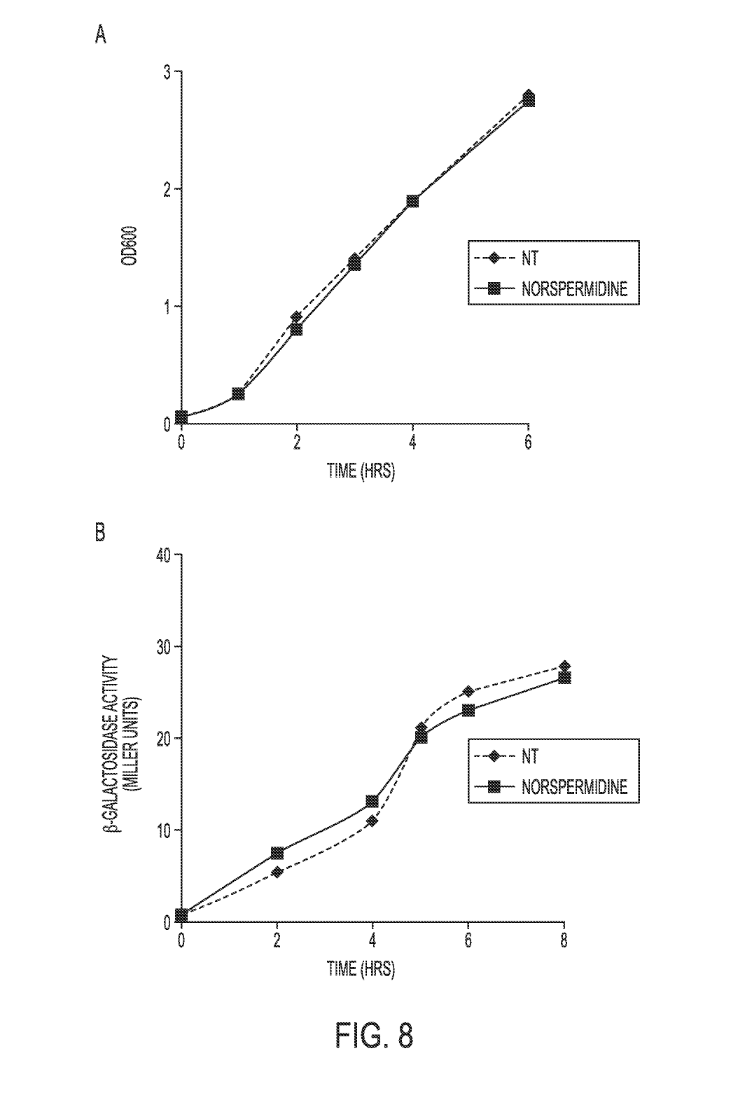

NCBI 3610 was grown in MSgg medium applied with norspermidine (100 μM) with shaking or in untreated medium served as a control (NT). The expression of the operons, epsA-O and yqxM-sipW-tasA, that specify the exopolysaccharide and protein components of the extracellular matrix, respectively, was not measurably impaired by the addition of norspermidine (FIGS. 8A & 8B). Also, cell growth was not significantly inhibited by norspermidine (FIGS. 8A & 8B). Therefore, whether norspermidine interacts directly with the exopolysaccharide was investigated. To attempt to detect such an interaction, dynamic light scattering was used. Dynamic scattering is a standard procedure for measuring the average radius of polymers in which a laser beam is transmitted through a sample containing polymers in solution (Berne, 1976; Orgad et al., 2011; Vinayahan et al., 2010).

Exopolysaccharide was purified from pellicles. Light scattering was measured for exopolysaccharide alone as well as for exopolysaccharide that had been mixed with 0.75 mM norspermidine or with 0.75 mM spermidine. Shown in FIG. 7A are the results obtained in the absence of polyamine (black), in the presence of norspermidine (white), and in the presence of spermidine (grey) with exopolysaccharide at the indicated concentrations and pH. Error bars represent the standard deviation of polymer radii among the polymers in a single sample. FIG. 7A shows that purified exopolysaccharide exhibited an average radius of 585±40 nm at pH 5.5, presumably representing an effective radius for the interacting linear polymers. Strikingly, treatment of the exopolysaccharide with norspermidine reduced the average radius substantially (175±10 nm) whereas treatment with spermidine had only a small effect on the average radius (500±20 nm). This indicates that the specificity of norspermidine resulted from a direct interaction with exopolysaccharide. The effect of norspermidine was seen over a range of exopolysaccharide concentrations (1-30 mg/ml) and also at pH 7 (FIG. 7A).

As an independent approach to detecting an interaction between norspermidine and exopolysaccharide, scanning electron microscopy was also carried out. Purified exopolysaccharide was dissolved in double distilled water at a final concentration of 10 mg/ml and mixed with either norspermidine or spermidine (0.75 mM final concentration). Samples were prepared as described. FIG. 7B shows three different magnifications of representative fields showing exopolysaccharide alone (EPS) and exopolysaccharide that had been mixed with norspermidine (EPS+norspermidine) or with spermidine (EPS+spermidine). FIGS. 8A and 8B show that controls had little effect on growth or eps transcription. Purified exopolysaccharide was seen to be in the form of aggregates, which had an average size of ˜570 nm (FIG. 7B). Strikingly, the addition of norspermidine reduced the size of the aggregates to ˜85 nm (FIG. 7B). Once again, and, as a demonstration of specificity, spermidine had little effect on the size of the aggregates.

### Example 6

### Small Molecule Screening for Biofilm-Inhibitory Activity

To identify features of norspermidine important for its biofilm disassembly activity, we tested a library of polyamines in our biofilm inhibition assay (FIGS. 9A, 9B, 10 and Table 2). In addition to norspermidine (1), we found that norspermine (2) exhibited biofilm-inhibitory activity against B. subtilis. These molecules have in common a motif consisting of three methylene groups flanked by two amino groups. The motif is present twice in 1 and three times in 2. Another polyamine, 1,3-diaminopropane (6), has only one copy of the motif and was less active (>5 mM). Also relatively inactive (inhibition was only observed at concentrations above 2 mM) were spermidine (3), spermine (4) and putrescine (5), which have a pair of amines separated by four methylenes, and cadaverine (19), which has a pair of amines separated by 5 methylenes. Replacing some or all of the amines in norspermidine with tertiary amines (7-9, 18), replacing the secondary amine with an ether linkage (10), or eliminating two or all of the amines (20, 11) resulted molecules that were relatively inactive. Whereas replacing the terminal amines with tertiary amines resulted in inactivity, in one case (21) the presence of a tertiary amine at the middle position did not block activity. The charge of each amine (at the neutral pH of the medium) was also important for biofilm inhibiting activity. Molecules that had neutral amide bonds instead of amines separated by three methylenes (15-17) were only weakly active or inactive (FIG. 10 and Table 2).

TABLE 2

| | | MBIC- |
|---|---|---|
| | | Minimal |
| | | Biofilm |
| | | Inhibitory |
| | | Concentration |
| Molecule | Formula | (μM) |
| 1 | 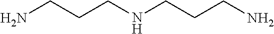 norspermidine | 25-50 |
| 2 | 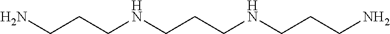 [SMILES: NCCCNCCCNCCCN] norspermine | 200 |
| 3 | 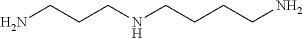 spermidine | 5000 |
| 4 | 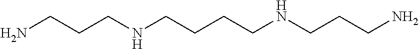 [SMILES: NCCCNCCCCNCCCN] spermine | 2500 |
| 5 | 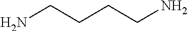 | 2000-3000 |
| 6 | 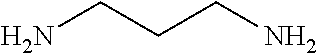 | 2500-5000 |
| 7 | 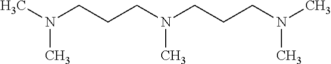 | >5000 |
| 8 | 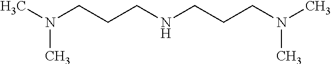 | >5000 |
| 9 | 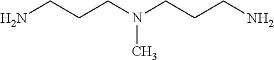 | >5000 |
| 10 | 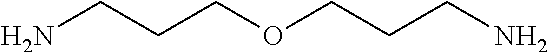 | 5000 |
| 11 | 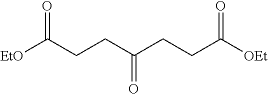 [SMILES: CCOC(=O)CCC(=O)CCC(=O)OCC] | >5000 |
| 12 | 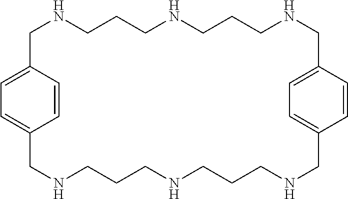 [SMILES: c1cc2ccc1CNCCCNCCCNCc1ccc(cc1)CNCCCNCCCNC2] | 500 |
| 13 | 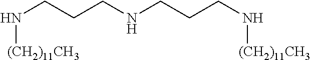 | 100 |
| 14 | 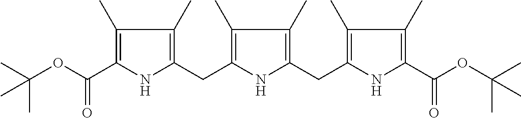 [SMILES: Cc1c(Cc2[nH]c(C(=O)OC(C)(C)C)c(C)c2C)[nH]c(Cc2[nH]c(C(=O)OC(C)(C)C)c(C)c2C)c1C] | 500-700 |
| 15 | 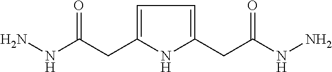 [SMILES: NNC(=O)Cc1ccc(CC(=O)NN)[nH]1] | >5000 |
| 16 | 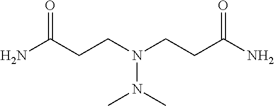 | >5000 |
| 17 | 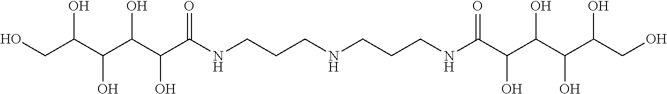 [SMILES: O=C(NCCCNCCCNC(=O)C(O)C(O)C(O)C(O)CO)C(O)C(O)C(O)C(O)CO] | >5000 |
| 18 | 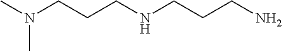 | >5000 |
| 19 | 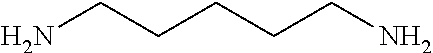 | 3000 |
| 20 | 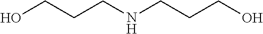 | >5000 |
| 21 | 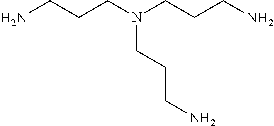 [SMILES: NCCCN(CCCN)CCCN] | 200 |

We conclude that a structure consisting of three methylene groups flanked by two positively charged amino groups contributes to biofilm-inhibiting activity. Reinforcing this hypothesis, three additional, synthetic polyamines exhibiting this motif (12, 13 and 14) were also active (FIG. 10).

FIGS. 9C, 9D, and 11 illustrate the computer modeling of the interaction of norspermidine and spermidine with an acidic exopolysaccharide. Norspermidine binds via salt bridges between amino and carboxyl groups (dotted lines) in a clamp-like mode across the exopolysaccharide secondary structure of a disaccharide repeat [α(1,6)Glc-β(1,3)GlcA]ₙ. Whereas norspermidine aligns well with the repeat, the spacing of amino groups of spermidine does not match the symmetric pattern of anionic side groups, implying weaker affinity.

### Example 7

### Evaluation of Norspermidine and Spermidine for Inhibiting Biofilm Formation by S. aureus and E. coli

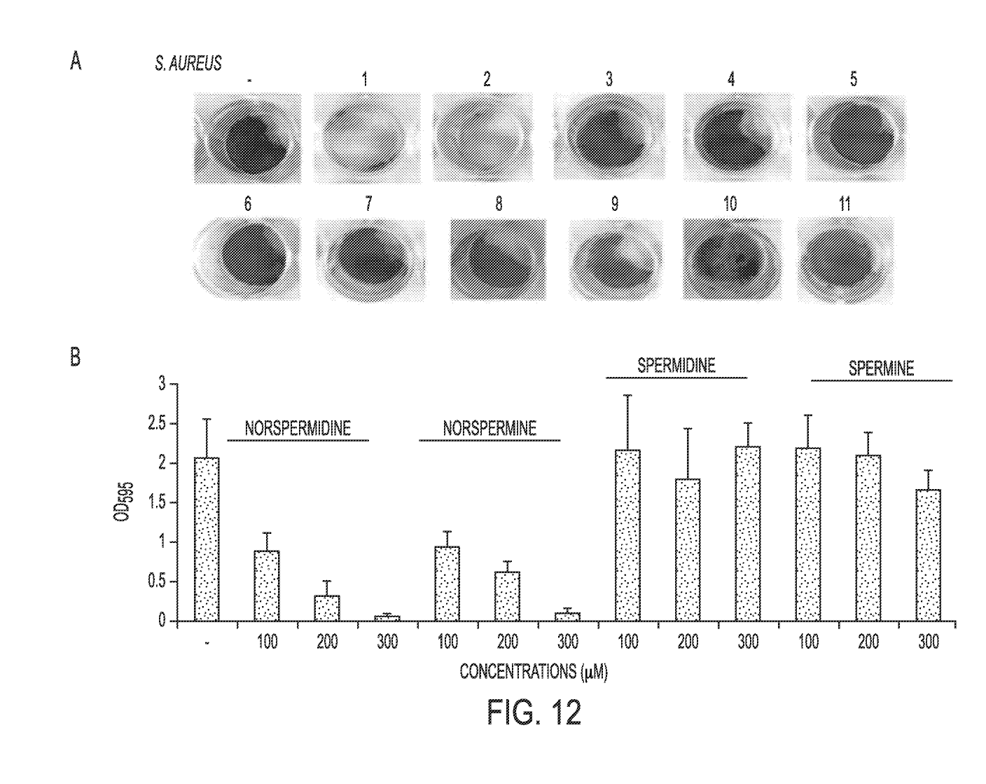

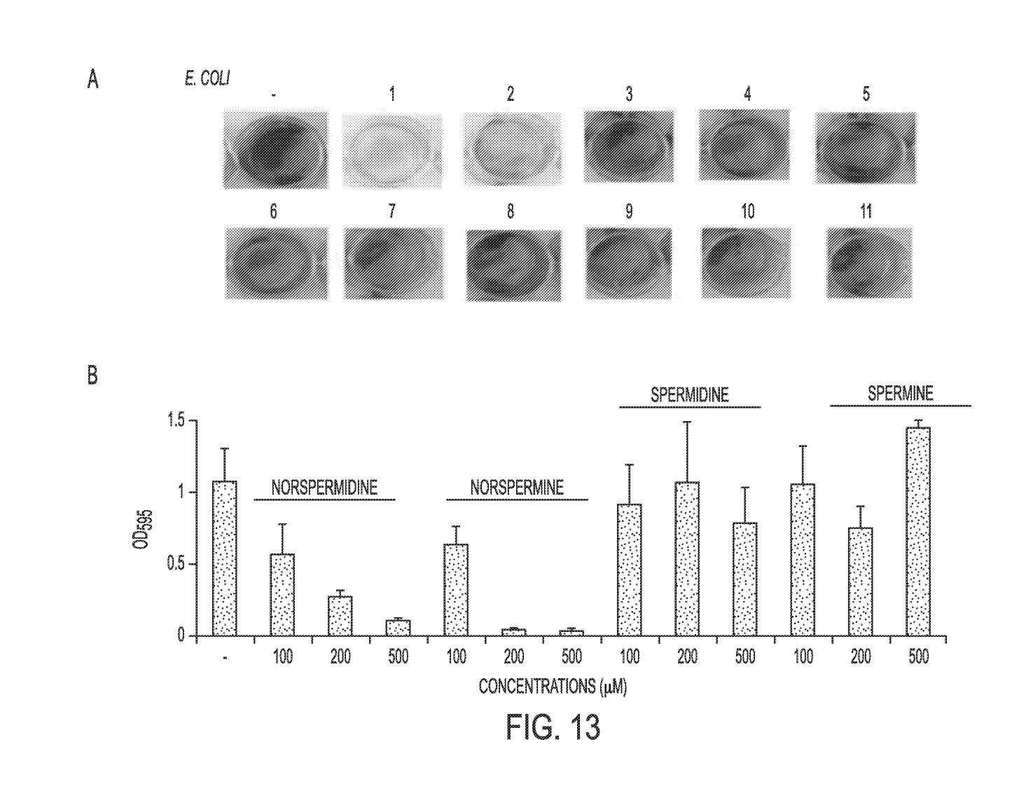

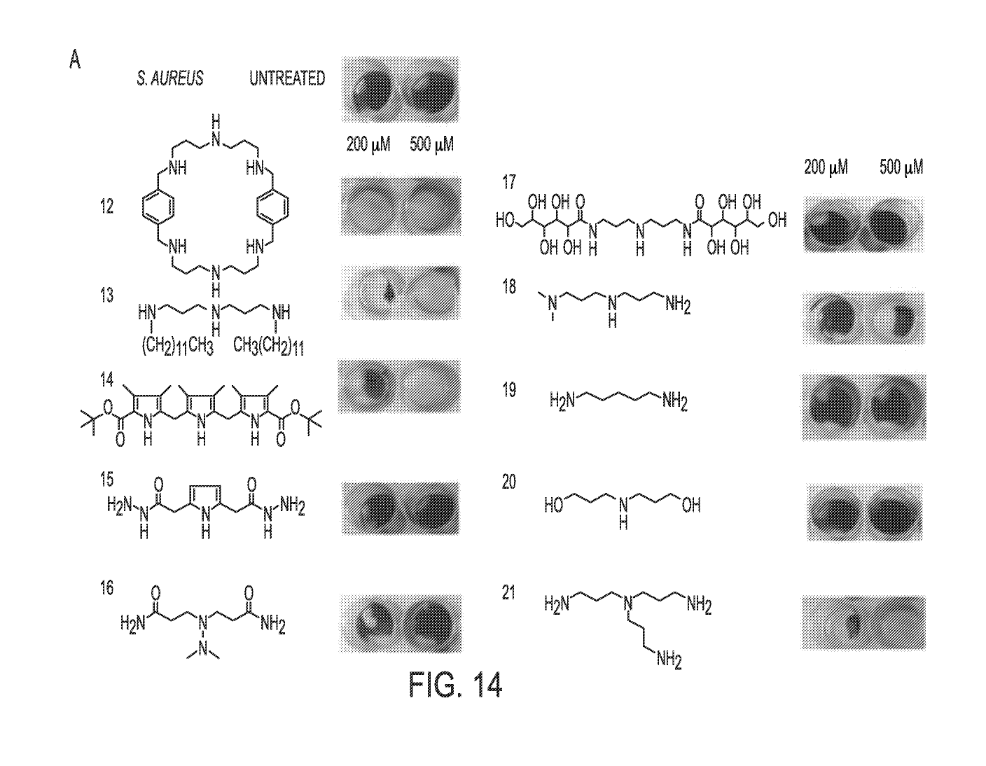

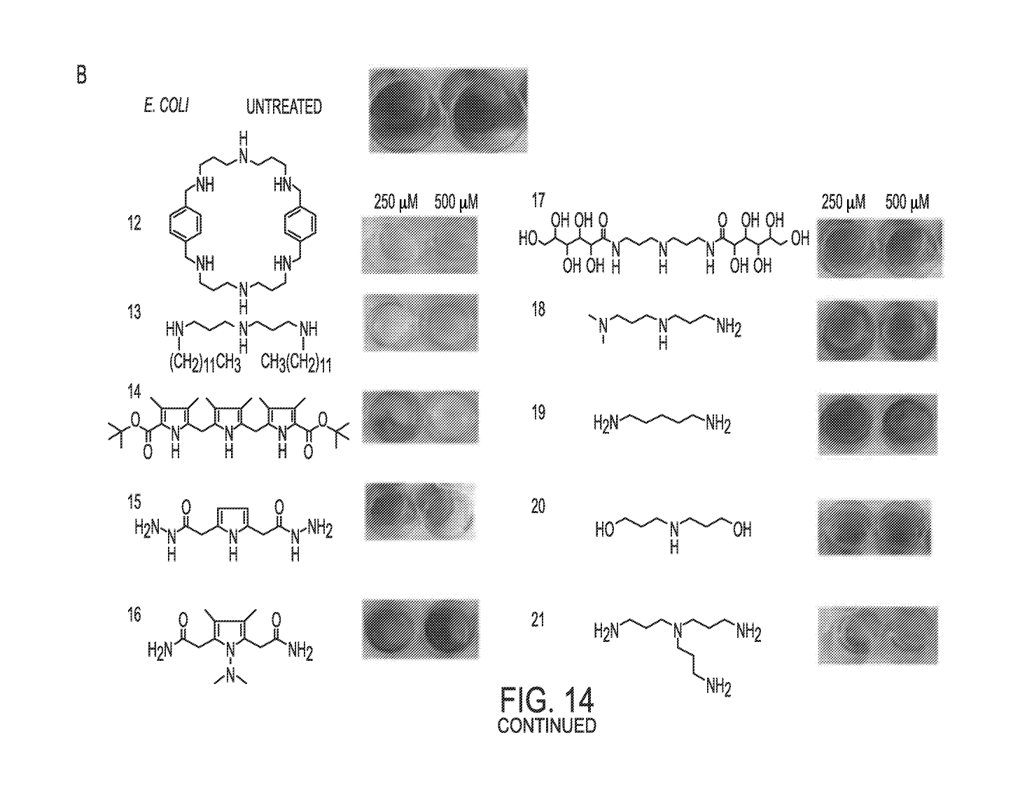

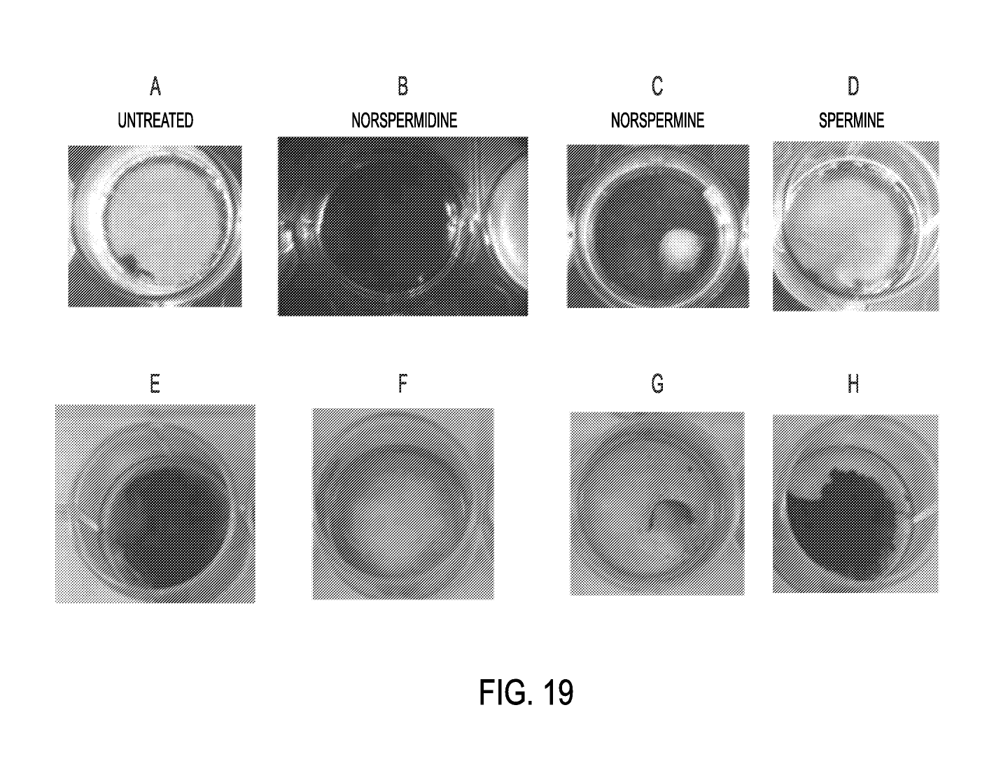

Whether polyamines might prevent biofilm formation by other bacteria that produce an exopolysaccharide matrix was examined. Indeed, the same molecules that inhibited biofilm formation by B. subtilis were effective in inhibiting biofilm formation by Staphylococcus aureus and Escherichia coli whereas those that were inactive with B. subtilis were similarly not effective (FIGS. 12A & 12B, 13A & 13B, 14A & 14B, and FIGS. 19A-H). FIG. 12A shows the effect of the numbered compounds displayed in FIG. 9A on the formation of submerged biofilms by S. aureus strain SCO1. The compounds were tested at 500 μM. Biofilm formation was visualized by crystal violet staining of submerged biofilms.

Similarly, FIGS. 19A-H show the results of S. aureus strain SC-01 grown to a mid-logarithmic phase and diluted 1:1000 into a 12 wells plate in a TSB medium applied with NaCl (3%) and Glucose (0.5%). The medium was applied with norspermidine (10 μM), norspermine (100 μM) or spermine (500 μM). Planktonic cells were removed (19A-D). Wells were washed once, and incubated with Crystal Violet (0.1%) for 15 mins. Wells were washed twice in DDW (19E-H). As can be seen in FIGS. 19B, 19C, 19F, and 19G, norspermidine and norspermine inhibit biofilm formation.

In testing E. coli, the biofilm-proficient strain MC4100 was selected because a major component of exopolysacchride is colanic acid (Danese et al., 2000; Price and Raivio, 2009). Colanic acid is a negatively charged polymer, and light scattering experiments indicated a direct interaction with norspermidine. Reinforcing the idea that norspermidine was targeting the exopolysaccharide, fluorescence microscopy experiments analogous to those presented above for B. subtilis showed markedly diminished staining of exopolysaccharide when cells of S. aureus and E. coli were treated with norspermidine but not spermidine.

### Example 8

### Screening of Polyamines in Biofilm Formation in B. Subtilis

B. subtilis wild-type cells were grown to a mid-logarithmic phase and 1 μl of cells was mixed with 1 μl (1 mM) of each polyamine. The mixture was plated on solid biofilm medium.

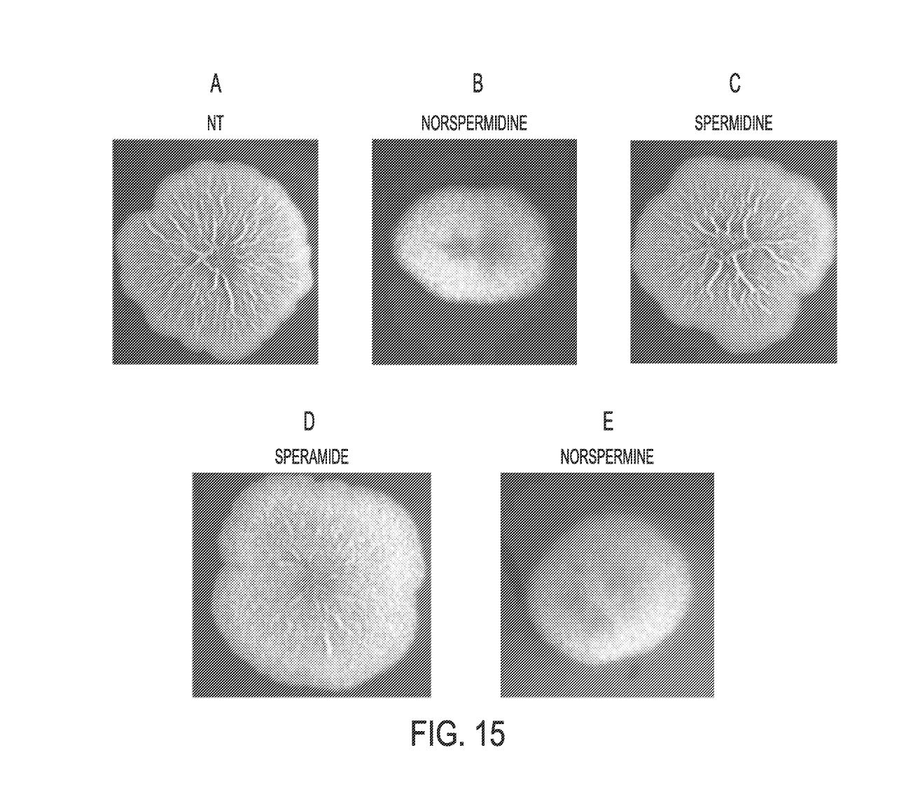

As can be seen in FIGS. 15B and 15E, norspermidine and norspermine inhibit biofilm formation. As seen in FIGS. 15C and 15D, spermidine and speramide do not.

### Example 9

### Screening of Polyamines in Pellicle Formation in B. Subtilis

The effect of polyamines on pellicle formation by B. subtilis was examined. B. subtilis wild-type cells were grown to a mid-logarithmic phase and diluted 1:1000 in biofilm media applied with each polyamine to the final concentration of 50 μM.

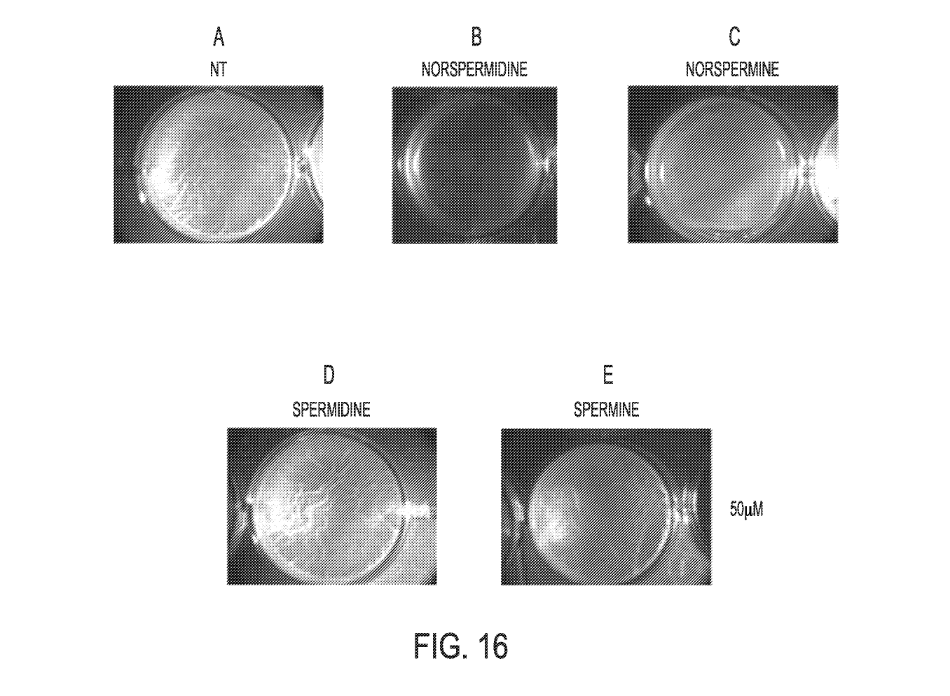

As can be seen in FIGS. 16B and 16C, norspermidine and norspermine inhibit biofilm formation. As seen in FIGS. 16D and 16E, spermidine and speramide do not.

### Example 10

### Screening of Polyamines for Disassembly of Pellicle Formation by B. Subtilis

Whether polyamines could trigger disassembly of biofilms formed by B. subtilis was examined. B. subtilis wild-type cells grown in liquid biofilm medium. Cells were grown to mid-logarithmic phase and diluted 1:1000 in liquid biofilm media. At day 2, pre-formed pellicles were applied with either PBS (A) or norspermine (250 μM) (B). Pellicles were incubated for additional 24 hrs.

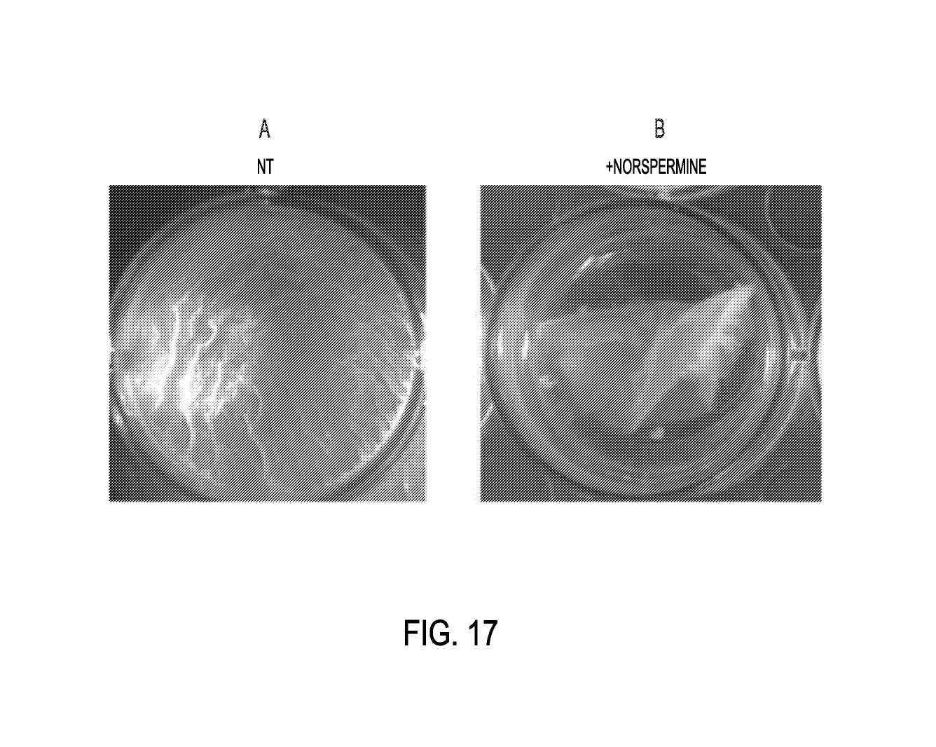

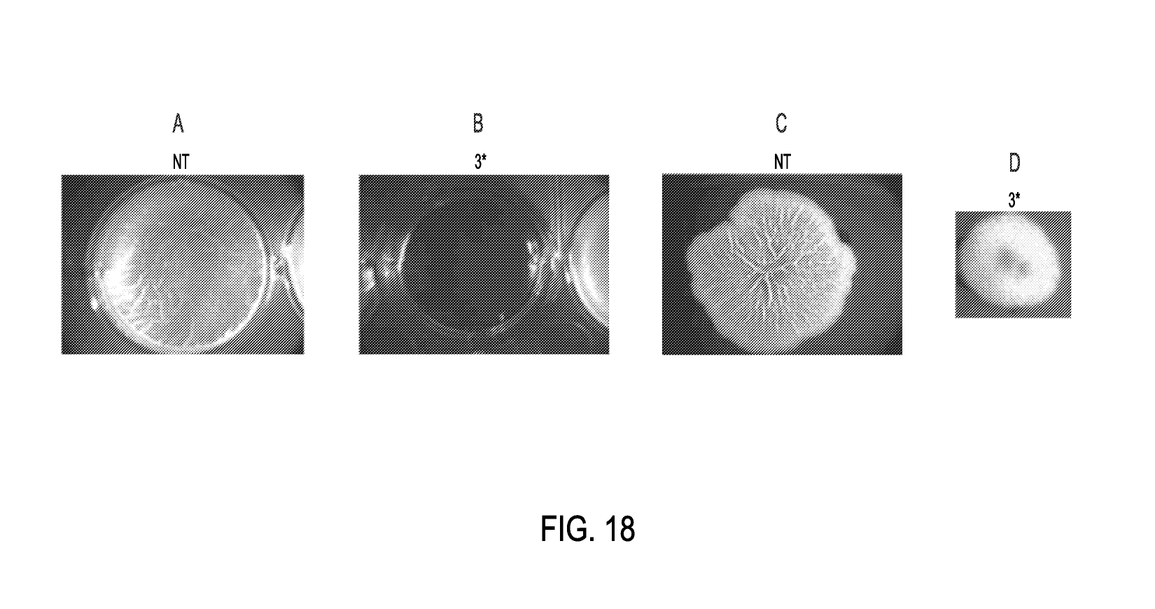

As can be seen in FIG. 17B, norspermine triggered disassembly of the pellicles.

### Example 11

### Screening of 1,5,7-Triazbicyclo[4,4,0]dec-5-ene for Biofilm Formation by B. subtilis

The effect of cyclic polyamines on biofilm formation by B. subtilis was examined. B. subtilis wild-type cells were grown to a mid-logarithmic phase and diluted 1:1000 in biofilm media (18A) or in a medium applied with cyclic compound 1,5,9-Triazacyclododecane to the final concentration of 50 μM (18B).

B. subtilis wild-type cells were grown to a mid-logarithmic phase and 1 μl of cells was either mixed with PBS (18C) or mixed with 1 μl (1 mM) of each polyamine (18D). The mixture was plated on solid biofilm medium. “3*” refers to 1,5,9-Triazacyclododecane.

As can be seen in 18B and 18D, biofilm formation was inhibited.

### Example 12

### Screening of Polyamines Combined with D-Amino Acids in Staphylococcus

The effect of certain polyamines combined with biofilm-inhibiting D-amino acids in Staphylococcus was examined. S. aureus strain SC-01 was grown to a mid-logarithmic phase and diluted 1:1000 into a 12 wells plate in a TSB medium applied with NaCl (3%) and Glucose (0.5%). The medium was applied with either norspermidine or D-tyrosine or both as indicated below each panel. Planktonic cells were removed Wells were washed once, and incubated with Crystal Violet (0.1%) for 15 mins. Wells were washed twice in DDW.

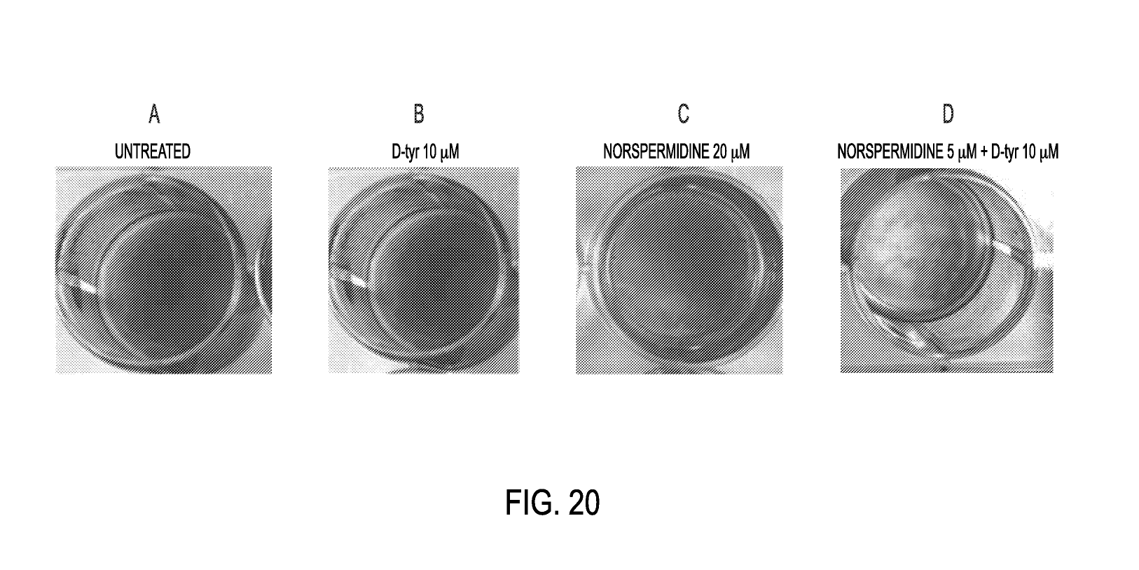

As can be seen in FIG. 20D, the combination of norspermidine and D-tyrosine acted synergistically to inhibit biofilm formation.

### Example 13

### Screening of Polyamines in Biofilm Formation in Pseudomonas

The effect of polyamines on biofilm formation by Pseudomonas was examined. P. aeriginosa strain PA14 were grown to a mid-logarithmic phase and 1 μl of cells was mixed with 1 μl (10 mM) of each polyamine. The mixture was plated on solid M63 medium.

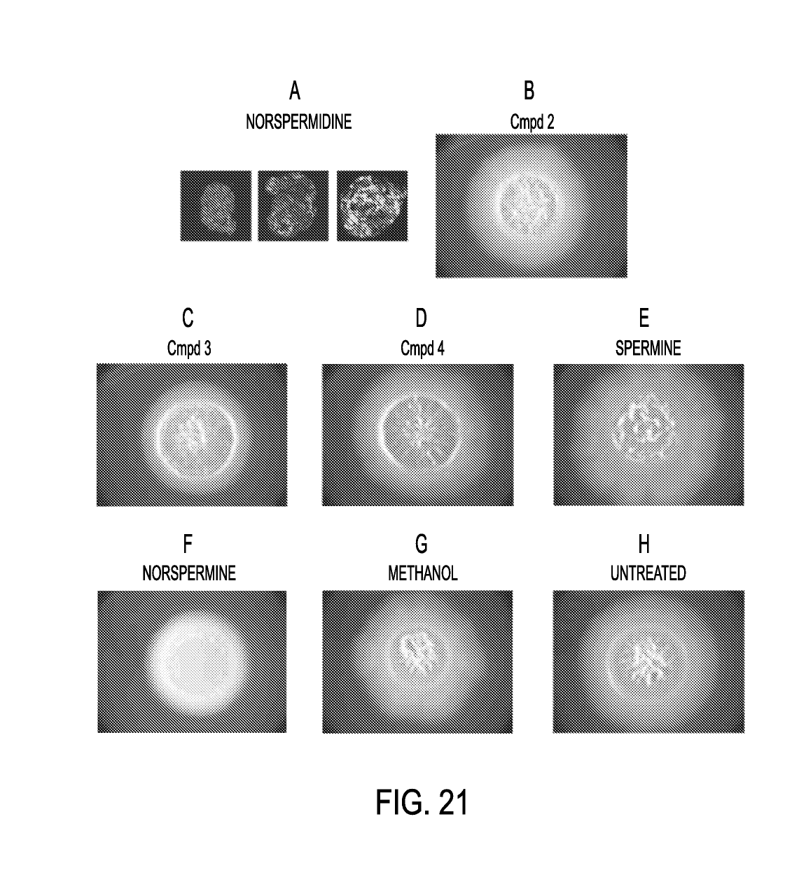

As can be seen in FIGS. 21A and 21F, norspermidine and norspermine inhibit biofilm formation.

### Example 14

### Inhibition of Proteus mirabilis Biofilm Formation by Norspermidine and Norspermine

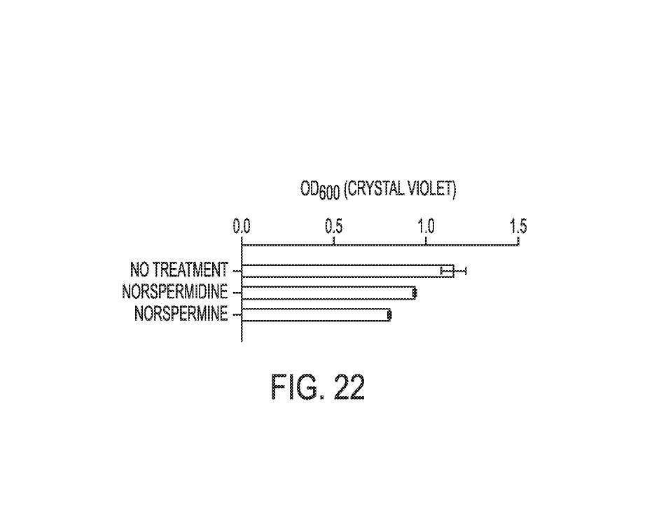

Clinically derived Proteus mirabilis strain BB2000 can form robust biofilms in multi-well polystyrene cell culture dishes and that these biofilms may be partially inhibited by 1 mM norspermidine and norspermine. To examine the effects of polyamines on biofilm formation, wells were initially treated with water (no treatment), 1 mM norspermidine, and 1 mM norspermine in 3 ml M9+glucose before inoculation with P. mirabilis. Biofilms were permitted to grow for 48 hours at 30° C. without shaking The amount of biofilm formed was assessed with the standard assay of crystal violet staining (O'Toole G et al., Biofilm formation as microbial development, Annu Rev. Microbiol. (2000) 54:49-79); supernatant was removed from the wells and crystal violet (at 200 ml) was then incubated in each well for 15 minutes at room temperature, followed by removal of the crystal violet from the wells and a subsequent washes with water. Any crystal violet attached the wells was next solubilized with 95% ethanol for 15 minutes at room temperature, and the subsequent optical density at 595 nm in each well was measured with a multiplate reader. The addition of either 1 mM norspermidine or 1 mM norspermine was sufficient to reduce biofilm formation in comparison to the no treatment control (see figure). These results suggest that polyamines act to partially inhibit the formation of mature Proteus biofilms on polystyrene plastic surfaces. The results are shown in FIG. 22.

### Example 15

### Synthesis of Certain Amines

Certain amines of Formula (II) can be synthesized from the corresponding amides by reduction with LiAlH₄ (see e.g.: Annenkov, Synthesis of biomimetic polyamines, (2009) ARKIVOC (xiii) 116-130.). Polypropylamide, synthesized by ring opening polymerization of β-alanine N-carboxyanhydride can be reduced by BH₃SMe₂ in THF under reflux to yield the corresponding polypropylamine (Fischer, Synthesis of Linear Polyamines with Different Amine Spacings and their Ability to Form dsDNA/siRNA Complexes Suitable for Transfection, (2010) Macromol. Biosci. 10(9): 1073-1083). Another exemplary method is the hydroaminomethylation of an alkene with a primary or secondary amine with CO/H₂ and a catalyst (e.g. [Rh(cod)Cl]₂) in dioxane or toluene. Primary amines may be protected as phthalimides which are finally deprotected by hydrazinolysis in ethanol (Muller, Synthesis of polyamines via hydroaminomethylation of alkenes with urea—a new, effective and versatile route to dendrons and dendritic core molecules, (2006) Org. Biomol. Chem. 4: 826-835). Analogs of norspermine can be produced by coupling of 1,3-dibromopropanes with 1,3-diaminopropane (Kneifel, Occurrence of norspermine in Euglena gracilis, (1978) Biochem Biophys Res Comm 85(1): 42-46). Synthesis of amines of formula (II) also can be achieved from alcohols by a one-pot conversion to amines using sodium azide and triphenylphosphine in CCl₄/DMF (Reddy, A New Novel and Practical One-Pot Methodology for Conversion of Alcohols to Amines, (2000) Synth. Commun. 30(12): 2233-2237). Furthermore, norspermidine derivatives can be prepared by general synthetic methods for conversion of primary amines to secondary amines with Raney nickel using linear or branched alkyldiamines (Lee, Diamine and Triamine Analogs and Derivatives as Inhibitors of Deoxyhypusine Synthase: Synthesis and Biological Activity, (1995) J. Med. Chem. 38: 3053-3061).

### EQUIVALENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of treating, reducing, or inhibiting biofilm formation by bacteria, the method comprising:
contacting an article with a composition comprising an effective amount of a polyamine having:
(a) Formula (I),
HR₇N-(M-N(R₇)—)ₓ-M-N(R₇)—Y—N(R₇)-(M-N(R₇)—)ₓ-M-NR₇H (I)
wherein
M is —C(R₁R₂)C(R₃R₄)C(R₅R₆)—;
each R₁, R₂, R₃, R₄, R₅, and R₆ is H, C₁-C₁₂ alkyl, C₁-C₁₂ alkenyl, C₂-C₁₂ alkynyl, C₁-C₁₂ alkoxy, alkaryl, aryl, or heteroaryl, so that each R₁, R₂, R₃, R₄, R₅, and R₆ may be the same or different;
each R₇ is H, C₁-C₁₆ alkyl, C₂-C₁₆ alkenyl, C₂-C₁₆ alkynyl, aryl, heteroaryl, or C₇₋₂₂ aralkyl, so that each R₇ may be the same or different;
Y is a moiety that interrupts the polyamine chain and is C₁-C₁₂ alkyl, C₁-C₁₂ alkenyl, C₂-C₁₂ alkynyl, C₁-C₁₂ alkoxy, alkaryl, aryl, or heteroaryl, polymer block, or oligomer block; and
each x is greater than or equal to 1, or
(b) Formula (II),
Hₐ-[R₇N-(L-N(R₈)—)ₓ-L-NH—P_{b}]_{c} (II),
wherein
each P is R₇ or Q or L;
each L is M or —C(R₁R₂)—X—C(R₅R₆)— or 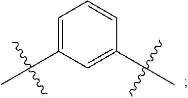 [SMILES: *c1cccc(*)c1]
each M is —C(R₁R₂)C(R₃R₄)C(R₅R₆)—;
each R₁, R₂, R₃, R₄, R₅, and R₆ is H, C₁-C₁₂ alkyl, C₁-C₁₂ alkenyl, C₂-C₁₂ alkynyl, C₁-C₁₂ alkoxy, alkaryl, aryl, or heteroaryl, so that each R₁, R₂, R₃, R₄, R₅, and R₆ may be the same or different;
each R₇ is H, C₁-C₁₆ alkyl, C₂-C₁₆ alkenyl, C₂-C₁₆ alkynyl, aryl, heteroaryl, or C₇₋₂₂ aralkyl which may be substituted on the aryl ring, or R₈ so that each R₇ may be the same or different, wherein R₇ is R₈ when the polyamine is cyclic;
each R₈ is H, C₁₋₈ ω-amino alkyl, C₂₋₈ ω-amino alkenyl, C₂₋₈ ω-amino alkynyl, amino alkaryl, amino aryl, N-heteroaryl;
each X is —NH—, —O—, —N(R₈)—, or S;
each Q is C₁₋₃₀ alkyl, C₂₋₃₀ alkenyl, C₂₋₃₀ alkynyl, alkaryl, aryl, heteroaryl, which may be substituted and where the alkyl, alkenyl, alkynyl, and alkaryl may be interrupted by one or more heteroatoms such as N, O, or S;
a is 0 or 1, wherein when a is 0 the polyamine is cyclic and when a is 1 the polyamine is linear or branched;
each b is 0 or 1;
c is greater than or equal to 1; and
each x is greater than or equal to 1,
thereby treating, reducing, or inhibiting formation of the biofilm.

2. The method of claim 1, wherein the polyamine has Formula (IIa),
H₂N-(M-NH)ₓ-M-NH₂ (IIa).

3. The method of claim 1, wherein the polyamine has Formula (IIb),

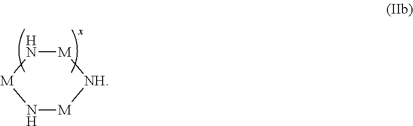

4. The method of claim 1, wherein the polyamine is a compound or combination of compounds from the following:

| Com- | |
|---|---|
| pound | Formula |
| a | 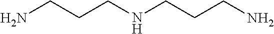 Norspermidine (also known as N-(3-aminopropyl)propane- 1,3-diamine) |
| b | 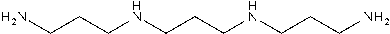 [SMILES: NCCCNCCCNCCCN] Norspermine |
| c | 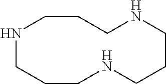 [SMILES: C1CNCCCNCCCNC1] 1,5,9-triazacyclododecane |
| d | 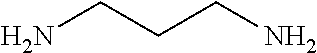 1,3-diaminopropane |
| e | 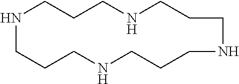 [SMILES: C1CNCCCNCCCNCCCNC1] 1,5,9,13-tetraazacyclohexadecane |
| f | 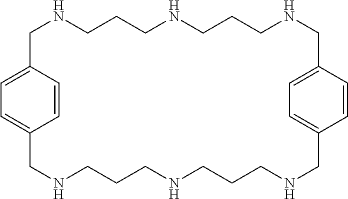 [SMILES: c1cc2ccc1CNCCCNCCCNCc1ccc(cc1)CNCCCNCCCNC2] 3,7,11,18,22,26-Hexaazatricyclo[26.2.2.213,16]tetratriaconta- 13,15,28,30,31,33-hexaene |
| g | 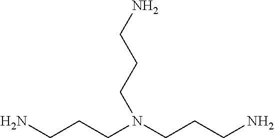 [SMILES: NCCCN(CCCN)CCCN] N¹,N¹-bis(3-aminopropyl)propane-1,3-diamine |
| h | 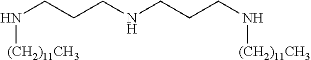 N1-dodecyl-N3-(3-(dodecylamino)propyl)propane-1,3-diamine |
| i | 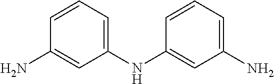 [SMILES: Nc1cccc(Nc2cccc(N)c2)c1] N,N-Di(3-aminophenyl)amine |

5. The method of claim 1, wherein the composition further comprises an effective amount of a D-amino acid, thereby treating, reducing or inhibiting formation of the biofilm.

6. The method of claim 5, wherein the D-amino acid is selected from the group consisting of D-alanine, D-cysteine, D-aspartic acid, D-glutamic acid, D-histidine, D-isoleucine, D-lysine, D-leucine, D-asparagine, D-proline, D-glutamine, D-arginine, D-serine, D-threonine, D-valine, D-tryptophan, D-tyrosine, and combinations thereof.

7. The method of claim 1, wherein the article is one or more selected from the group consisting of comprises a industrial equipment, plumbing systems, bodies of water, household surfaces, textiles and paper.

8. The method of claim 1, wherein the article is made from a material selected from the group consisting of metal, metal alloy, synthetic polymer, natural polymer, ceramic, wood, glass, leather, paper, fabric, non-metallic inorganics, composite materials and combinations thereof.

9. The method of claim 1, wherein contacting comprises applying a coating to the article, said coating comprising an effective amount of the polyamine.

10. The method of claim 9, wherein the coating further comprises a binder.

11. The method of claim 1, wherein contacting comprises introducing a polyamine into a precursor material and processing the precursor material into the article impregnated with the polyamine.

12. The method of claim 1, further comprising contacting the surface with a biocide.

13. A coated article resistant to biofilm formation, comprising:
an article comprising a coating on at least one exposed surface, the coating comprising an effective amount of a polyamine of:
(a) Formula (I),
HR₇N-(M-N(R₇)—)ₓ-M-N(R₇)—Y—N(R₇)-(M-N(R₇)—)ₓ-M-NR₇H (I)
wherein
M is —C(R₁R₂)C(R₃R₄)C(R₅R₆)—;
each R₁, R₂, R₃, R₄, R₅, and R₆ is H, C₁-C₁₂ alkyl, C₁-C₁₂ alkenyl, C₂-C₁₂ alkynyl, C₁-C₁₂ alkoxy, alkaryl, aryl, or heteroaryl, so that each R₁, R₂, R₃, R₄, R₅, and R₆ may be the same or different;
each R₇ is H, C₁-C₁₆ alkyl, C₂-C₁₆ alkenyl, C₂-C₁₆ alkynyl, aryl, heteroaryl, or C₇₋₂₂ aralkyl, so that each R₇ may be the same or different;
Y is a moiety that interrupts the polyamine chain and is C₁-C₁₂ alkyl, C₁-C₁₂ alkenyl, C₂-C₁₂ alkynyl, C₁-C₁₂ alkoxy, alkaryl, aryl, or heteroaryl, polymer block, or oligomer block; and
each x is greater than or equal to 1, or
(b) Formula (II),
Hₐ-[R₇N-(L-N(R₈)—)ₓ-L-NH—P_{b}]_{c} (II),
wherein
each P is R₇ or Q or L;
each L is M or —C(R₁R₂)—X—C(R₅R₆)— or 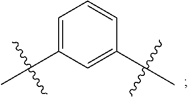 [SMILES: *c1cccc(*)c1]
each M is —C(R₁R₂)C(R₃R₄)C(R₅R₆)—;
each R₁, R₂, R₃, R₄, R₅, and R₆ is H, C₁-C₁₂ alkyl, C₁-C₁₂ alkenyl, C₂-C₁₂ alkynyl, C₁-C₁₂ alkoxy, alkaryl, aryl, or heteroaryl, so that each R₁, R₂, R₃, R₄, R₅, and R₆ may be the same or different;
each R₇ is H, C₁-C₁₆ alkyl, C₂-C₁₆ alkenyl, C₂-C₁₆ alkynyl, aryl, heteroaryl, or C₇₋₂₂ aralkyl which may be substituted on the aryl ring, or R₈ so that each R₇ may be the same or different, wherein R₇ is R₈ when the polyamine is cyclic;
each R₈ is H, C₁₋₈ ω-amino alkyl, C₂₋₈ ω-amino alkenyl, C₂₋₈ ω-amino alkynyl, amino alkaryl, amino aryl, N-heteroaryl;
each X is —NH—, —O—, —N(R₈)—, or S;
each Q is C₁₋₃₀ alkyl, C₂₋₃₀ alkenyl, C₂₋₃₀ alkynyl, alkaryl, aryl, heteroaryl, which may be substituted and where the alkyl, alkenyl, alkynyl, and alkaryl may be interrupted by one or more heteroatoms such as N, O, or S;
a is 0 or 1, wherein when a is 0 the polyamine is cyclic and when a is 1 the polyamine is linear or branched;
each b is 0 or 1;
c is greater than or equal to 1; and
each x is greater than or equal to 1,
thereby treating, reducing or inhibiting formation of the biofilm.

14. The coated article of claim 13, wherein the polyamine has Formula (IIa),
H₂N-(M-NH)ₓ-M-NH₂ (IIa).

15. The coated article of claim 13, wherein the polyamine has Formula (IIb),

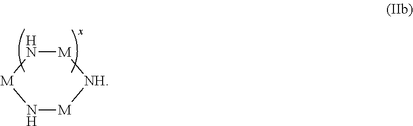

16. The coated article of claim 13, wherein the polyamine is a compound or combination of compounds from the following:

| Com- | |
|---|---|
| pound | Formula |
| a | 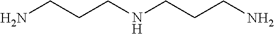 Norspermidine (also known as N-(3-aminopropyl)propane- 1,3-diamine) |
| b | 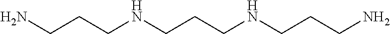 [SMILES: NCCCNCCCNCCCN] Norspermine |
| c | 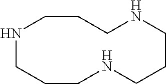 [SMILES: C1CNCCCNCCCNC1] 1,5,9-triazacyclododecane |
| d | 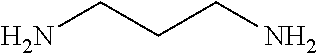 1,3-diaminopropane |
| e | 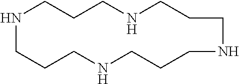 [SMILES: C1CNCCCNCCCNCCCNC1] 1,5,9,13-tetraazacyclohexadecane |
| f | 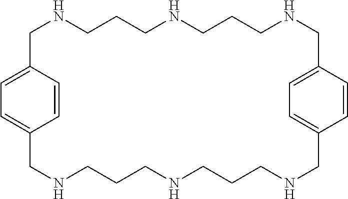 [SMILES: c1cc2ccc1CNCCCNCCCNCc1ccc(cc1)CNCCCNCCCNC2] 3,7,11,18,22,26-Hexaazatricyclo[26.2.2.213,16]tetratriaconta- 13,15,28,30,31,33-hexaene |
| g | 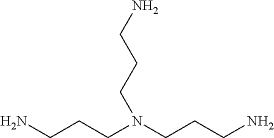 [SMILES: NCCCN(CCCN)CCCN] N¹,N¹-bis(3-aminopropyl)propane-1,3-diamine |
| h | 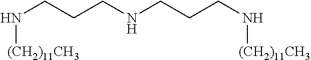 N1-dodecyl-N3-(3-(dodecylamino)propyl)propane-1,3-diamine |
| i | 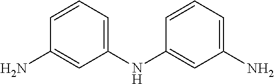 [SMILES: Nc1cccc(Nc2cccc(N)c2)c1] N,N-Di(3-aminophenyl)amine |

17. The coated article of claim 13, wherein the article is one or more components involved in water condensate collection, water recirculation, sewerage transport, paper pulping and manufacture, and water processing and transport.

18. A composition resistant to biofilm formation, comprising:
a fluid base or a polymeric binder; and
an effective amount of a polyamine of
(a) Formula (I),
HR₇N-(M-N(R₇)—)ₓ-M-N(R₇)—Y—N(R₇)-(M-N(R₇)—)ₓ-M-NR₇H (I)
wherein
M is —C(R₁R₂)C(R₃R₄)C(R₅R₆)—;
each R₁, R₂, R₃, R₄, R₅, and R₆ is H, C₁-C₁₂ alkyl, C₁-C₁₂ alkenyl, C₂-C₁₂ alkynyl, C₁-C₁₂ alkoxy, alkaryl, aryl, or heteroaryl, so that each R₁, R₂, R₃, R₄, R₅, and R₆ may be the same or different;
each R₇ is H, C₁-C₁₆ alkyl, C₂-C₁₆ alkenyl, C₂-C₁₆ alkynyl, aryl, heteroaryl, or C₇₋₂₂ aralkyl, so that each R₇ may be the same or different;
Y is a moiety that interrupts the polyamine chain and is C₁-C₁₂ alkyl, C₁-C₁₂ alkenyl, C₂-C₁₂ alkynyl, C₁-C₁₂ alkoxy, alkaryl, aryl, or heteroaryl, polymer block, or oligomer block; and
each x is greater than or equal to 1, or
(b) Formula (II),
Hₐ-[R₇N-(L-N(R₈)—)ₓ-L-NH—P_{b}]_{c} (II),
wherein
each P is R₇ or Q or L;
each L is M or —C(R₁R₂)—X—C(R₅R₆)— or 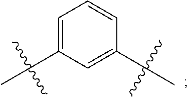 [SMILES: *c1cccc(*)c1]
each M is —C(R₁R₂)C(R₃R₄)C(R₅R₆)—;
each R₁, R₂, R₃, R₄, R₅, and R₆ is H, C₁-C₁₂ alkyl, C₁-C₁₂ alkenyl, C₂-C₁₂ alkynyl, C₁-C₁₂ alkoxy, alkaryl, aryl, or heteroaryl, so that each R₁, R₂, R₃, R₄, R₅, and R₆ may be the same or different;
each R₇ is H, C₁-C₁₆ alkyl, C₂-C₁₆ alkenyl, C₂-C₁₆ alkynyl, aryl, heteroaryl, or C₇₋₂₂ aralkyl which may be substituted on the aryl ring, or R₈ so that each R₇ may be the same or different, wherein R₇ is R₈ when the polyamine is cyclic;
each R₈ is H, C₁₋₈ ω-amino alkyl, C₂₋₈ ω-amino alkenyl, C₂₋₈ ω-amino alkynyl, amino alkaryl, amino aryl, N-heteroaryl;
each X is —NH—, —O—, —N(R₈)—, or S;
each Q is C₁₋₃₀ alkyl, C₂₋₃₀ alkenyl, C₂₋₃₀ alkynyl, alkaryl, aryl, heteroaryl, which may be substituted and where the alkyl, alkenyl, alkynyl, and alkaryl may be interrupted by one or more heteroatoms such as N, O, or S;
a is 0 or 1, wherein when a is 0 the polyamine is cyclic and when a is 1 the polyamine is linear or branched;
each b is 0 or 1;
c is greater than or equal to 1; and
each x is greater than or equal to 1,
said polyamine being distributed in the base or polymeric binder,
thereby treating, reducing or inhibiting formation of the biofilm.

19. The composition of claim 18, wherein the composition comprises a fluid base selected from a liquid, gel, paste.

20. The composition of claim 18, wherein the composition comprises coating composition comprising a polymeric binder.
